SEQUENCE LISTING (169 sequences)

SEQ ID NO: 1 - DNA, 1440 bp, Escherichia coli
atgtcagtacccgttcaacatcctatgtatatcgatggacagtttgttacctggcgtggagacgcatggattgatgtggtaaaccctgctacagaggctgtcatttcccgcatacccgatggtcaggccgaggatgcccgtaaggcaatcgatgcagcagaacgtgcacaaccagaatgggaagcgttgcctgctattgaacgcgccagttggttgcgcaaaatctccgccgggatccgcgaacgcgccagtgaaatcagtgcgctgattgttgaagaagggggcaagatccagcagctggctgaagtcgaagtggcttttactgccgactatatcgattacatggcggagtgggcacggcgttacgagggcgagattattcaaagcgatcgtccaggagaaaatattcttttgtttaaacgtgcgcttggtgtgactaccggcattctgccgtggaacttcccgttcttcctcattgcccgcaaaatggctcccgctcttttgaccggtaataccatcgtcattaaacctagtgaatttacgccaaacaatgcgattgcattcgccaaaatcgtcgatgaaataggccttccgcgcggcgtgtttaaccttgtactggggcgtggtgaaaccgttgggcaagaactggcgggtaacccaaaggtcgcaatggtcagtatgacaggcagcgtctctgcaggtgagaagatcatggcgactgcggcgaaaaacatcaccaaagtgtgtctggaattggggggtaaagcaccagctatcgtaatggacgatgccgatcttgaactggcagtcaaagccatcgttgattcacgcgtcattaatagtgggcaagtgtgtaactgtgcagaacgtgtttatgtacagaaaggcatttatgatcagttcgtcaatcggctgggtgaagcgatgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttcggtggcaaagcggtagaggggaaaggatattattatccgccgacattgctgctggatgttcgccaggaaatgtcgattatgcatgaggaaacctttggcccggtgctgccagttgtcgcatttgacacgctggaagatgctatctcaatggctaatgacagtgattacggcctgacctcatcaatctatacccaaaatctgaacgtcgcgatgaaagccattaaagggctgaagtttggtgaaacttacatcaaccgtgaaaacttcgaagctatgcaaggcttccacgccggatggcgtaaatccggtattggcggcgcagatggtaaacatggcttgcatgaatatctgcagacccaggtggtttatttacagtcttaa

SEQ ID NO: 2 - DNA, 1539 bp, Escherichia coli
atgaccaataatcccccttcagcacagattaagcccggcgagtatggtttccccctcaagttaaaagcccgctatgacaactttattggcggcgaatgggtagcccctgccgacggcgagtattaccagaatctgacgccggtgaccgggcagctgctgtgcgaagtggcgtcttcgggcaaacgagacatcgatctggcgctggatgctgcgcacaaagtgaaagataaatgggcgcacacctcggtgcaggatcgtgcggcgattctgtttaagattgccgatcgaatggaacaaaacctcgagctgttagcgacagctgaaacctgggataacggcaaacccattcgcgaaaccagtgctgcggatgtaccgctggcgattgaccatttccgctatttcgcctcgtgtattcgggcgcaggaaggtgggatcagtgaagttgatagcgaaaccgtggcctatcatttccatgaaccgttaggcgtggtggggcagattatcccgtggaacttcccgctgctgatggcgagctggaaaatggctcccgcgctggcggcgggcaactgtgtggtgctgaaacccgcacgtcttaccccgctttctgtactgctgctaatggaaattgtcggtgatttactgccgccgggcgtggtgaacgtggtcaatggcgcaggtggggtaattggcgaatatctggcgacctcgaaacgcatcgccaaagtggcgtttaccggctcaacggaagtgggccaacaaattatgcaatacgcaacgcaaaacattattccggtgacgctggagttgggcggtaagtcgccaaatatcttctttgctgatgtgatggatgaagaagatgcctttttcgataaagcgctggaaggctttgcactgtttgcctttaaccagggcgaagtttgcacctgtccgagtcgtgctttagtgcaggaatctatctacgaacgctttatggaacgcgccatccgccgtgtcgaaagcattcgtagcggtaacccgctcgacagcgtgacgcaaatgggcgcgcaggtttctcacgggcaactggaaaccatcctcaactacattgatatcggtaaaaaagagggcgctgacgtgctcacaggcgggcggcgcaagctgctggaaggtgaactgaaagacggctactacctcgaaccgacgattctgtttggtcagaacaatatgcgggtgttccaggaggagatttttggcccggtgctggcggtgaccaccttcaaaacgatggaagaagcgctggagctggcgaacgatacgcaatatggcctgggcgcgggcgtctggagccgcaacggtaatctggcctataagatggggcgcggcatacaggctgggcgcgtgtggaccaactgttatcacgcttacccggcacatgcggcgtttggtggctacaaacaatcaggtatcggtcgcgaaacccacaagatgatgctggagcattaccagcaaaccaagtgcctgctggtgagctactcggataaaccgttggggctgttctga

SEQ ID NO: 3 - DNA, 1473 bp, Escherichia coli
atgtcccgaatggcagaacagcagctttatatacatggtggttatacctccgccaccagcggtcgcaccttcgagaccattaacccggccaacggtaacgtgctggcgaccgtgcaggccgccgggcgcgaggatgtcgatcgcgccgtgaaaagcgcccagcaggggcaaaaaatctgggcgtcgatgaccgccatggagcgctcgcgtattctgcgtcgggccgttgatattctgcgtgaacgcaatgacgaactcgcaaaactggaaaccctcgacaccggaaaagcatattcggaaacctcaaccgtcgatatcgttaccggtgcggacgtgctggagtactacgccgggctgatcccggcgctggaaggcagccagatcccgttgcgtgaaacgtcctttgtgtatacccgccgcgaaccgctgggcgtagtggcagggattggcgcatggaactacccgatccagattgccctgtggaaatccgccccggcgctggcggcaggcaacgcaatgattttcaaaccgagcgaagttaccccgcttaccgcgttaaagctggctgaaatttacagcgaagcgggcctgccggacggcgtatttaacgtgttgccgggcgtgggcgcggagaccgggcaatatctgaccgagcatccgggcattgccaaagtgtcatttaccggcggtgtcgccagcggcaaaaaagtgatggctaactcggcggcctcttccctgaaagaagtgaccatggaactgggcggtaaatcaccgctgatcgttttcgatgatgcggatctcgatctcgccgccgatatcgccatgatggcaaacttcttcagctccggtcaggtgtgtaccaatggcacccgcgtcttcgttccggcgaaatgcaaagccgcatttgagcagaaaattctggcgcgcgttgagcgcattcgcgcgggcgacgttttcgatccgcaaactaacttcggcccgctggtcagcttcccgcatcgcgataacgtgctgcgctatatcgccaaaggcaaagaggaaggcgcgcgcgtactgtgcggcggcgatgtactgaaaggcgatggcttcgataacggcgcatgggttgcaccgacagtgttcaccgattgcagcgacgatatgaccatcgtgcgtgaagagatcttcgggccagtgatgtccattctgacctacgagtcggaagacgaagtcattcgccgcgctaacgataccgactacggcctggcggcgggcatcgtgacagcggacctgaaccgcgcgcatcgcgtcattcatcagctggaagcgggtatttgctggatcaacacctggggcgaatccccggcagagatgcccgttggcggctacaaacactccggcattggtcgcgagaacggcgtgatgacgctccagagttacacccaggtgaagtccatccaggttgagatggctaaattccagtccatattctaa

SEQ ID NO: 4 - DNA, 1404 bp, Escherichia coli
atgaatcaacaggatattgaacaggtggtgaaagcggtactgctgaaaatgcaaagcagtgacacgccgtccgccgccgttcatgagatgggcgttttcgcgtccctggatgacgccgttgcggcagccaaagtcgcccagcaagggttaaaaagcgtggcaatgcgccagttagccattgctgccattcgtgaagcaggcgaaaaacacgccagagatttagcggaacttgccgtcagtgaaaccggcatggggcgcgttgaagataaatttgcaaaaaacgtcgctcaggcgcgcggcacaccaggcgttgagtgcctctctccgcaagtgctgactggcgacaacggcctgaccctaattgaaaacgcaccctggggcgtggtggcttcggtgacgccttccactaacccggcggcaaccgtaattaacaacgccatcagcctgattgccgcgggcaacagcgtcatttttgccccgcatccggcggcgaaaaaagtctcccagcgggcgattacgctgctcaaccaggcgattgttgccgcaggtgggccggaaaacttactggttactgtggcaaatccggatatcgaaaccgcgcaacgcttgttcaagtttccgggtatcggcctgctggtggtaaccggcggcgaagcggtagtagaagcggcgcgtaaacacaccaataaacgtctgattgccgcaggcgctggcaacccgccggtagtggtggatgaaaccgccgacctcgcccgtgccgctcagtccatcgtcaaaggcgcttctttcgataacaacatcatttgtgccgacgaaaaggtactgattgttgttgatagcgtagccgatgaactgatgcgtctgatggaaggccagcacgcggtgaaactgaccgcagaacaggcgcagcagctgcaaccggtgttgctgaaaaatatcgacgagcgcggaaaaggcaccgtcagccgtgactgggttggtcgcgacgcaggcaaaatcgcggcggcaatcggccttaaagttccgcaagaaacgcgcctgctgtttgtggaaaccaccgcagaacatccgtttgccgtgactgaactgatgatgccggtgttgcccgtcgtgcgcgtcgccaacgtggcggatgccattgcgctagcggtgaaactggaaggcggttgccaccacacggcggcaatgcactcgcgcaacatcgaaaacatgaaccagatggcgaatgctattgataccagcattttcgttaagaacggaccgtgcattgccgggctggggctgggcggggaaggctggaccaccatgaccatcaccacgccaaccggtgaaggggtaaccagcgcgcgtacgtttgtccgtctgcgtcgctgtgtattagtcgatgcgtttcgcattgtttaa

SEQ ID NO: 5 - DNA, 1188 bp, Escherichia coli
atgcaaaatgaattgcagaccgcgctctttcaggcgttcgataccctgaatctgcaacgggtaaaaacatttagcgttccaccggtgacgctttgcggtccgggctcggtgagcagttgcggacagcaagcgcaaacgcgtgggctgaaacatctgttcgtgatggcagacagctttttgcatcaggcagggatgaccgccgggctgacgcgtagcctgaccgttaaaggtatcgccatgacgctctggccatgtccggtgggcgaaccgtgcattaccgacgtgtgtgcagccgtggcgcagttgcgtgagtcaggctgtgatggggtgatcgcgtttggcggcggctcggtgctggatgcggcgaaagccgtgacgttgctggtgacgaacccggatagcacgctggcagagatgtcagaaaccagcgttctgcaaccgcgcttgccgctgattgccattccaactaccgccggaaccggctctgaaaccaccaatgtaacggtgattatcgacgcggtgagcgggcgcaagcaggtgttagcccatgcctcgctgatgccggatgtggcgatcctcgacgccgcattgaccgaaggtgtgccgtcgcatgtcacggcgatgaccggcattgatgcgttaacccatgccattgaagcatacagcgccctgaacgctacaccgtttaccgacagtctggcgattggtgccattgcgatgattggcaaatcgctgccgaaagcggtgggctacggtcacgaccttgccgcgcgcgagagcatgttgctggcttcatgtatggcgggaatggcgttttccagtgcgggtcttgggttgtgccacgcgatggcgcatcagccgggcgcggcgctgcatattccgcacggtctcgcgaacgccatgttgctgccaacggtgatggaatttaaccggatggtttgtcgtgaacgctttagtcagattggtcgggcactgcgaactaaaaaatccgacgatcgtgacgctattaacgcggtaagtgagctgattgcggaagttgggattggtaaacgactgggcgatgttggtgcgacatctgcgcattacggcgcatgggcgcaggccgcgctggaagatatttgtctgcgcagtaacccgcgtaccgccagcctggagcagattgtcggcctgtacgcagcggcgcaataa

SEQ ID NO: 6 - DNA, 1152 bp, Escherichia coli
atgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaagggctttccccgaccaataaacccagtgtaccgattctggcaattcctaccacagcaggtactgcggcagaagtgaccattaactacgtgatcactgacgaagagaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttataacactccacacggtgttgcgaacgccatcctgttaccgcatgtcatgcgttataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggcgcaggcggcactggatgatgtttgtaccggtggcaacccgcgtgaagcaacgcttgaggatattgtagagctttaccataccgcctggtaa

SEQ ID NO: 7 - DNA, 1449 bp, Escherichia coli
atgaaacttaacgacagtaacttattccgccagcaggcgttgattaacggggaatggctggacgccaacaatggtgaagccatcgacgtcaccaatccggcgaacggcgacaagctgggtagcgtgccgaaaatgggcgcggatgaaacccgcgccgctatcgacgccgccaaccgcgccctgcccgcctggcgcgcgctcaccgccaaagaacgcgccaccattctgcgcaactggttcaatttgatgatggagcatcaggacgatttagcgcgcctgatgaccctcgaacagggtaaaccactggccgaagcgaaaggcgaaatcagctacgccgcctcctttattgagtggtttgccgaagaaggcaaacgcatttatggcgacaccattcctggtcatcaggccgataaacgcctgattgttatcaagcagccgattggcgtcaccgcggctatcacgccgtggaacttcccggcggcgatgattacccgcaaagccggtccggcgctggcagcaggctgcaccatggtgctgaagcccgccagtcagacgccgttctctgcgctggcgctggcggagctggcgatccgcgcgggcgttccggctggggtatttaacgtggtcaccggttcggcgggcgcggtcggtaacgaactgaccagtaacccgctggtgcgcaaactgtcgtttaccggttcgaccgaaattggccgccagttaatggaacagtgcgcgaaagacatcaagaaagtgtcgctggagctgggcggtaacgcgccgtttatcgtctttgacgatgccgacctcgacaaagccgtggaaggcgcgctggcctcgaaattccgcaacgccgggcaaacctgcgtctgcgccaaccgcctgtatgtgcaggacggcgtgtatgaccgttttgccgaaaaattgcagcaggcagtgagcaaactgcacatcggcgacgggctggataacggcgtcaccatcgggccgctgatcgatgaaaaagcggtagcaaaagtggaagagcatattgccgatgcgctggagaaaggcgcgcgcgtggtttgcggcggtaaagcgcacgaacgcggcggcaacttcttccagccgaccattctggtggacgttccggccaacgccaaagtgtcgaaagaagagacgttcggccccctcgccccgctgttccgctttaaagatgaagctgatgtgattgcgcaagccaatgacaccgagtttggccttgccgcctatttctacgcccgtgatttaagccgcgtcttccgcgtgggcgaagcgctggagtacggcatcgtcggcatcaataccggcattatttccaatgaagtggccccgttcggcggcatcaaagcctcgggtctgggtcgtgaaggttcgaagtatggcatcgaagattacttagaaatcaaatatatgtgcatcggtctttaa

SEQ ID NO: 8 - DNA, 891 bp, Escherichia coli
atgactatgaaagttggttttattggcctggggattatgggtaaaccaatgagtaaaaaccttctgaaagcaggttactcgctggtggttgctgaccgtaacccagaagctattgctgacgtgattgctgcaggtgcagaaacagcgtctacggctaaagcgatcgctgaacagtgcgacgtcatcataaccatgctgccaaactcccctcatgtgaaagaggtggcgctgggtgagaatggcattattgaaggcgcgaagccaggtacggtattgatcgatatgagttctatcgcaccgctggcaagccgtgaaatcagcgaagcgctgaaagcgaaaggcattgatatgctggatgctccggtgagcggcggtgaaccgaaagccatcgacggtacgctgtcagtgatggtgggcggcgacaaggctattttcgacaaatactatgatttgatgaaagcgatggcgggttccgtggtgcataccggggaaatcggtgcaggtaacgtcaccaaactggcaaatcaggtcattgtggcgctgaatattgccgcgatgtcagaagcgttaacgctggcaactaaagcgggcgttaacccggacctggtttatcaggcaattcgcggtggactggcgggcagtaccgtgctggatgccaaagcgccgatggtgatggaccgcaacttcaagccgggcttccgtattgatctgcatattaaggatctggcgaatgcgctggatacttctcacggcgtcggcgcacaactgccgctcacagctgcggttatggagatgatgcaggcactgcgagcagatggtttaggaacggcggatcatagcgccctggcgtgctactacgaaaaactggcgaaagtcgaagttactcgttaa

SEQ ID NO: 9 - DNA, 1104 bp, Escherichia coli
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaatgcgccggtggaggaaatcgaaaccgtagctgcccttagccatgcggtaggtttgccaataactctcgctcaactggatattaaagaagatgtcccggcgaaaatgcgaattgtggcagaagcggcatgtgcagaaggtgaaaccattcacaacatgcctggcggcgcgacgccagatcaggtttacgccgctctgctggtagccgaccagtacggtcagcgtttcctgcaagagtgggaataa

SEQ ID NO: 10 - DNA, 879 bp, Escherichia coli
atgaaactgggatttattggcttaggcattatgggtacaccgatggccattaatctggcgcgtgccggtcatcaattacatgtcacgaccattggaccggttgctgatgaattactgtcactgggtgccgtcagtgttgaaactgctcgccaggtaacggaagcatcggacatcatttttattatggtgccggacacacctcaggttgaagaagttctgttcggtgaaaatggttgtaccaaagcctcgctgaagggcaaaaccattgttgatatgagctccatttccccgattgaaactaagcgtttcgctcgtcaggtgaatgaactgggcggcgattatctcgatgcgccagtctccggcggtgaaatcggtgcgcgtgaagggacgttgtcgattatggttggcggtgatgaagcggtatttgaacgtgttaaaccgctgtttgaactgctcggtaaaaatatcaccctcgtgggcggtaacggcgatggtcaaacctgcaaagtggcaaatcagattatcgtggcgctcaatattgaagcggtttctgaagccctgctatttgcttcaaaagccggtgcggacccggtacgtgtgcgccaggcgctgatgggcggctttgcttcctcacgtattctggaagttcatggcgagcgtatgattaaacgcacctttaatccgggcttcaaaatcgctctgcaccagaaagatctcaacctggcactgcaaagtgcgaaagcacttgcgctgaacctgccaaacactgcgacctgccaggagttatttaatacctgtgcggcaaacggtggcagccagttggatcactctgcgttagtgcaggcgctggaattaatggctaaccataaactggcctga

SEQ ID NO: 11 - DNA, 1407 bp, Escherichia coli
atgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 12 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 13 - DNA, 2046 bp, Escherichia coli
atgcagcagttagccagtttcttatccggtacctggcagtctggccggggccgtagccgtttgattcaccacgctattagcggcgaggcgttatgggaagtgaccagtgaaggtcttgatatggcggctgcccgccagtttgccattgaaaaaggtgcccccgcccttcgcgctatgacctttatcgaacgtgcggcgatgcttaaagcggtcgctaaacatctgctgagtgaaaaagagcgtttctatgctctttctgcgcaaacaggcgcaacgcgggcagacagttgggttgatattgaaggtggcattgggacgttatttacttacgccagcctcggtagccgggagctgcctgacgatacgctgtggccggaagatgaattgatccccttatcgaaagaaggtggatttgccgcgcgccatttactgacctcaaagtcaggcgtggcagtgcatattaacgcctttaacttcccctgctggggaatgctggaaaagctggcaccaacgtggctgggcggaatgccagccatcatcaaaccagctaccgcgacggcccaactgactcaggcgatggtgaaatcaattgtcgatagtggtcttgttcccgaaggcgcaattagtctgatctgcggtagtgctggcgacttgttggatcatctggacagccaggatgtggtgactttcacggggtcagcggcgaccggacagatgctgcgagttcagccaaatatcgtcgccaaatctatccccttcactatggaagctgattccctgaactgctgcgtactgggcgaagatgtcaccccggatcaaccggagtttgcgctgtttattcgtgaagttgtgcgtgagatgaccacaaaagccgggcaaaaatgtacggcaatccggcggattattgtgccgcaggcattggttaatgctgtcagtgatgctctggttgcgcgattacagaaagtcgtggtcggtgatcctgctcaggaaggcgtgaaaatgggcgcactggtaaatgctgagcagcgtgccgatgtgcaggaaaaagtgaacatattgctggctgcaggatgcgagattcgcctcggtggtcaggcggatttatctgctgcgggtgccttcttcccgccaaccttattgtactgtccgcagccggatgaaacaccggcggtacatgcaacagaagcctttggccctgtcgcaacgctgatgccagcacaaaaccagcgacatgctctgcaactggcttgtgcaggcggcggtagccttgcgggaacgctggtgacggctgatccgcaaattgcgcgtcagtttattgccgacgcggcacgtacgcatgggcgaattcagatcctcaatgaagagtcggcaaaagaatccaccgggcatggctccccactgccacaactggtacatggtgggcctggtcgcgcaggaggcggtgaagaattaggcggtttacgagcggtgaaacattacatgcagcgaaccgctgttcagggtagtccgacgatgcttgccgctatcagtaaacagtgggtgcgcggtgcgaaagtcgaagaagatcgtattcatccgttccgcaaatattttgaggagctacaaccaggcgacagcctgttgactccccgccgcacaatgacagaggccgatattgttaactttgcttgcctcagcggcgatcatttctatgcacatatggataagattgctgctgccgaatctattttcggtgagcgggtggtgcatgggtattttgtgctttctgcggctgcgggtctgtttgtcgatgccggtgtcggtccggtcattgctaactacgggctggaaagcttgcgttttatcgaacccgtaaagccaggcgataccatccaggtgcgtctcacctgtaagcgcaagacgctgaaaaaacagcgtagcgcagaagaaaaaccaacaggtgtggtggaatgggctgtagaggtattcaatcagcatcaaaccccggtggcgctgtattcaattctgacgctggtggccaggcagcacggtgattttgtcgattaa

SEQ ID NO: 14 - DNA, 1254 bp, Escherichia coli
atgctggaacaaatgggcattgccgcgaagcaagcctcgtataaattagcgcaactctccagccgcgaaaaaaatcgcgtgctggaaaaaatcgccgatgaactggaagcacaaagcgaaatcatcctcaacgctaacgcccaggatgttgctgacgcgcgagccaatggccttagcgaagcgatgcttgaccgtctggcactgacgcccgcacggctgaaaggcattgccgacgatgtacgtcaggtgtgcaacctcgccgatccggtggggcaggtaatcgatggcggcgtactggacagcggcctgcgtcttgagcgtcgtcgcgtaccgctgggggttattggcgtgatttatgaagcgcgcccgaacgtgacggttgatgtcgcttcgctgtgcctgaaaaccggtaatgcggtgatcctgcgcggtggcaaagaaacgtgtcgcactaacgctgcaacggtggcggtgattcaggacgccctgaaatcctgcggcttaccggcgggtgccgtgcaggcgattgataatcctgaccgtgcgctggtcagtgaaatgctgcgtatggataaatacatcgacatgctgatcccgcgtggtggcgctggtttgcataaactgtgccgtgaacagtcgacaatcccggtgatcacaggtggtataggcgtatgccatatttacgttgatgaaagtgtagagatcgctgaagcattaaaagtgatcgtcaacgcgaaaactcagcgtccgagcacatgtaatacggttgaaacgttgctggtgaataaaaacatcgccgatagcttcctgcccgcattaagcaaacaaatggcggaaagcggcgtgacattacacgcagatgcagctgcactggcgcagttgcaggcaggccctgcgaaggtggttgctgttaaagccgaagagtatgacgatgagtttctgtcattagatttgaacgtcaaaatcgtcagcgatcttgacgatgccatcgcccatattcgtgaacacggcacacaacactccgatgcgatcctgacccgcgatatgcgcaacgcccagcgttttgttaacgaagtggattcgtccgctgtttacgttaacgcctctacgcgttttaccgacggcggccagtttggtctgggtgcggaagtggcggtaagcacacaaaaactccacgcgcgtggcccaatggggctggaagcactgaccacttacaagtggatcggcattggtgattacaccattcgtgcgtaa

SEQ ID NO: 15 - DNA, 3963 bp, Escherichia coli
atgggaaccaccaccatgggggttaagctggacgacgcgacgcgtgagcgtattaagtctgccgcgacacgtatcgatcgcacaccacactggttaattaagcaggcgattttttcttatctcgaacaactggaaaacagcgatactctgccggagctacctgcgctgctttctggcgcggccaatgagagcgatgaagcaccgactccggcagaggaaccacaccagccattcctcgactttgccgagcaaatattgccccagtcggtttcccgcgccgcgatcaccgcggcctatcgccgcccggaaaccgaagcggtttctatgctgctggaacaagcccgcctgccgcagccagttgctgaacaggcgcacaaactggcgtatcagctggccgataaactgcgtaatcaaaaaaatgccagtggtcgcgcaggtatggtccaggggttattgcaggagttttcgctgtcatcgcaggaaggcgtggcgctgatgtgtctggcggaagcgttgttgcgtattcccgacaaagccacccgcgacgcgttaattcgcgacaaaatcagcaacggtaactggcagtcacacattggtcgtagcccgtcactgtttgttaatgccgccacctgggggctgctgtttactggcaaactggtttccacccataacgaagccagcctctcccgctcgctgaaccgcattatcggtaaaagcggtgaaccgctgatccgcaaaggtgtggatatggcgatgcgcctgatgggtgagcagttcgtcactggcgaaaccatcgcggaagcgttagccaatgcccgcaagctggaagagaaaggtttccgttactcttacgatatgctgggcgaagccgcgctgaccgccgcagatgcacaggcgtatatggtttcctatcagcaggcgattcacgccatcggtaaagcgtctaacggtcgtggcatctatgaagggccgggcatttcaatcaaactgtcggcgctgcatccgcgttatagccgcgcccagtatgaccgggtaatggaagagctttacccgcgtctgaaatcactcaccctgctggcgcgtcagtacgatattggtatcaacattgacgccgaagagtccgatcgcctggagatctccctcgatctgctggaaaaactctgtttcgagccggaactggcaggctggaacggcatcggttttgttattcaggcttatcaaaaacgctgcccgttggtgatcgattacctgattgatctcgccacccgcagccgtcgccgtctgatgattcgcctggtgaaaggcgcgtactgggatagtgaaattaagcgtgcgcagatggacggccttgaaggttatccggtttatacccgcaaggtgtataccgacgtttcttatctcgcctgtgcgaaaaagctgctggcggtgccgaatctaatctacccgcagttcgcgacgcacaacgcccatacgctggcggcgatttatcaactggcggggcagaactactacccgggtcagtacgagttccagtgcctgcatggtatgggcgagccactgtatgagcaggtcaccgggaaagttgccgacggcaaacttaaccgtccgtgtcgtatttatgctccggttggcacacatgaaacgctgttggcgtatctggtgcgtcgcctgctggaaaacggtgctaacacctcgtttgttaaccgtattgccgacacctctttgccactggatgaactggtcgccgatccggtcactgctgtagaaaaactggcgcaacaggaagggcaaactggattaccgcatccgaaaattcccctgccgcgcgatctttacggtcacgggcgcgacaactcggcagggctggatctcgctaacgaacaccgcctggcctcgctctcctctgccctgctcaatagtgcactgcaaaaatggcaggccttgccaatgctggaacaaccggtagcggcaggtgagatgtcgcccgttattaaccctgcggaaccgaaagatattgtgggctatgtgcgtgaagccacgccgcgtgaagtagaacaggcgctggaaagtgcggttaataacgcgccaatctggtttgccacgcctccggctgaacgcgcagcgattttgcaccgcgctgccgtgctgatggaaagccagatgcagcaactgattggtattctggtgcgtgaggccggaaaaaccttcagtaacgccattgccgaagtgcgcgaagcggtcgattttctccactactacgccggacaggtgcgggatgatttcgctaacgaaacccaccgtccattagggcctgtggtgtgtatcagtccgtggaacttcccgctggctattttcaccgggcagatcgccgccgcactggcggcaggtaacagcgtgctggcaaaaccggcagaacaaacgccgctgattgccgcgcaagggatcgccattttgctggaagcgggtgtaccgccaggcgtggtgcaattgctgccaggtcggggtgaaaccgtgggcgcgcaactgacgggtgatgatcgcgtgcgcggggtgatgtttaccggttcaaccgaagtcgctacgttactgcagcgcaatatcgccagccgcctggacgctcagggtcgccctattccgctcatcgctgaaaccggcggcatgaacgcgatgattgtcgattcttcagcactgaccgaacaggtcgtcgtggatgtactggcctcggcgttcgacagtgcgggtcagcgttgttcggcgctgcgcgtgctgtgcctgcaagatgagattgccgaccacacgttgaaaatgctgcgcggcgcaatggccgaatgccggatgggtaatccgggtcgcctgaccaccgatatcggtccagtgattgatagcgaagcgaaagccaatattgagcgccatattcagaccatgcgtagcaaaggccgtccggtgttccaggcggtgcgggaaaacagcgaagatgcccgtgaatggcaaagcggcacctttgtcgccccgacgctgatcgaactggatgactttgccgaattgcaaaaagaggtctttggtccggtgctgcatgtggtgcgttacaaccgtaaccagctaccagagctgatcgagcagattaacgcttccggttatggtctgacgcttggcgtccatacgcgcattgatgaaaccatcgcccaggtcactggctcggcccatgttggtaacctgtatgttaaccgtaatatggtgggcgcagtggttggtgtgcagccgttcggcggcgaagggttgtccggtaccgggccgaaagcaggcggtccgctctatctctaccgtctgctggcgaatcgcccggaaagtgcgctggcagtgacgctcgcgcgtcaggatgcaaagtatccggtcgatgcgcagttgaaagccgcattgactcagccgctaaatgcactgcgggaatgggcagcaaatcgtccagaattgcaggcgttatgtacgcaatatggcgagctggcgcaggcaggaacacaacgattgctgccggggccgacgggtgaacgcaacacctggacgctgctgccgcgtgagcgcgtgttgtgtattgccgatgatgagcaggatgcgctgactcagctcgccgccgtgctggcggtgggcagccaggtactgtggccggatgacgcgctgcatcgtcagttagtgaaggcattgccatcggcagtcagcgaacgtattcaactggcgaaagcggaaaatataaccgctcaaccgtttgatgcggtgatcttccacggtgattcggatcagcttcgcgcattgtgtgaagcagttgccgcgcgggatggcacaattgtttcggtgcagggttttgcccgtggcgaaagcaatatccttctggaacggctgtatatcgagcgttcgctgagtgtgaataccgctgccgctggcggtaacgccagcttaatgactataggttaa

SEQ ID NO: 16 - DNA, 1488 bp, Escherichia coli
atgaattttcatcatctggcttactggcaggataaagcgttaagtctcgccattgaaaaccgcttatttattaacggtgaatatactgctgcggcggaaaatgaaacctttgaaaccgttgatccggtcacccaggcaccgctggcgaaaattgcccgcggcaagagcgtcgatatcgaccgtgcgatgagcgcagcacgcggcgtatttgaacgcggcgactggtcactctcttctccggctaaacgtaaagcggtactgaataaactcgccgatttaatggaagcccacgccgaagagctggcactgctggaaactctcgacaccggcaaaccgattcgtcacagtctgcgtgatgatattcccggcgcggcgcgcgccattcgctggtacgccgaagcgatcgacaaagtgtatggcgaagtggcgaccaccagtagccatgagctggcgatgatcgtgcgtgaaccggtcggcgtgattgccgccatcgtgccgtggaacttcccgctgttgctgacttgctggaaactcggcccggcgctggcggcgggaaacagcgtgattctaaaaccgtctgaaaaatcaccgctcagtgcgattcgtctcgcggggctggcgaaagaagcaggcttgccggatggtgtgttgaacgtggtgacgggttttggtcatgaagccgggcaggcgctgtcgcgtcataacgatatcgacgccattgcctttaccggttcaacccgtaccgggaaacagctgctgaaagatgcgggcgacagcaacatgaaacgcgtctggctggaagcgggcggcaaaagcgccaacatcgttttcgctgactgcccggatttgcaacaggcggcaagcgccaccgcagcaggcattttctacaaccagggacaggtgtgcatcgccggaacgcgcctgttgctggaagagagcatcgccgatgaattcttagccctgttaaaacagcaggcgcaaaactggcagccgggccatccacttgatcccgcaaccaccatgggcaccttaatcgactgcgcccacgccgactcggtccatagctttattcgggaaggcgaaagcaaagggcaactgttgttggatggccgtaacgccgggctggctgccgccatcggcccgaccatctttgtggatgtggacccgaatgcgtccttaagtcgcgaagagattttcggtccggtgctggtggtcacgcgtttcacatcagaagaacaggcgctacagcttgccaacgacagccagtacggccttggcgcggcggtatggacgcgcgacctctcccgcgcgcaccgcatgagccgacgcctgaaagccggttccgtcttcgtcaataactacaacgacggcgatatgaccgtgccgtttggcggctataagcagagcggcaacggtcgcgacaaatccctgcatgcccttgaaaaattcactgaactgaaaaccatctggataagcctggaggcctga

SEQ ID NO: 17 - DNA, 1389 bp, Escherichia coli
atgaccattactccggcaactcatgcaatttcgataaatcctgccacgggtgaacaactttctgtgctgccgtgggctggcgctgacgatatcgaaaacgcacttcagctggcggcagcaggctttcgcgactggcgcgagacaaatatagattatcgtgctgaaaaactgcgtgatatcggtaaggctctgcgcgctcgtagcgaagaaatggcgcaaatgatcacccgcgaaatgggcaaaccaatcaaccaggcgcgcgctgaagtggcgaaatcggcgaatttgtgtgactggtatgcagaacatggtccggcaatgctgaaggcggaacctacgctggtggaaaatcagcaggcggttattgagtatcgaccgttggggacgattctggcgattatgccgtggaattttccgttatggcaggtgatgcgtggcgctgttcccatcattcttgcaggtaacggctacttacttaaacatgcgccgaatgtgatgggctgtgcacagctcattgcccaggtgtttaaagatgcgggtatcccacaaggcgtatatggctggctgaatgccgacaacgacggtgtcagtcagatgattaaagactcgcgcattgctgctgtcacggtgaccggaagtgttcgtgcgggagcggctattggcgcacaggctggagcggcactgaaaaaatgcgtactggaactgggcggttcggatccgtttattgtgcttaacgatgccgatctggaactggcggtgaaagcggcggtagccggacgttatcagaataccggacaggtatgtgcagcggcaaaacgctttattatcgaagagggaattgcttcggcatttaccgaacgttttgtggcagctgcggcagccttgaaaatgggcgatccccgtgacgaagagaacgctctcggaccaatggctcgttttgatttacgtgatgagctgcatcatcaggtggagaaaaccctggcgcagggtgcgcgtttgttactgggcggggaaaagatggctggggcaggtaactactatccgccaacggttctggcgaatgttaccccagaaatgaccgcgtttcgggaagaaatgtttggccccgttgcggcaatcaccattgcgaaagatgcagaacatgcactggaactggctaatgatagtgagttcggcctttcagcgaccatttttaccactgacgaaacacaggccagacagatggcggcacgtctggaatgcggtggggtgtttatcaatggttattgtgccagcgacgcgcgagtggcctttggtggcgtgaaaaagagtggctttggtcgtgagctttcccatttcggcttacacgaattctgtaatatccagacggtgtggaaagaccggatctga

SEQ ID NO: 18 - DNA, 1146 bp, Escherichia coli
atgagtctgaatatgttctggtttttaccgacccacggtgacgggcattatctgggaacggaagaaggttcacgcccggttgatcacggttatctgcaacaaattgcgcaagcggcggatcgtcttggctataccggtgtgctaattccaacggggcgctcctgcgaagatgcgtggctggttgccgcatcgatgatcccggtgacgcagcggctgaagtttcttgtcgccctgcgtcccagcgtaacctcacctaccgttgccgcccgccaggccgccacgcttgaccgtctctcaaatggacgtgcgttgtttaacctggtcacaggcagcgatccacaagagctggcaggcgacggagtgttccttgatcatagcgagcgctacgaagcctcggcggaatttacccaggtctggcggcgtttattgcagagagaaaccgtcgatttcaacggtaaacatattcatgtgcgcggagcaaaactgctcttcccggcgattcaacagccgtatccgccactttactttggcggatcgtcagatgtcgcccaggagctggcggcagaacaggttgatctctacctcacctggggcgaaccgccggaactggttaaagagaaaatcgaacaagtgcgggcgaaagctgccgcgcatggacgcaaaattcgtttcggtattcgtctgcatgtgattgttcgtgaaactaacgacgaagcgtggcaggccgccgagcggttaatctcgcatcttgatgatgaaactatcgccaaagcacaggccgcattcgcccggacggattccgtagggcaacagcgaatggcggcgttacataacggcaagcgcgacaatctggagatcagccccaatttatgggcgggcgttggcttagtgcgcggcggtgccgggacggcgctggtgggcgatggtcctacggtcgctgcgcgaatcaacgaatatgccgcgcttggcatcgacagttttgtgctttcgggctatccgcatctggaagaagcgtatcgggttggcgagttgctgttcccgcttctggatgtcgccatcccggaaattccccagccgcagccgctgaatccgcaaggcgaagcggtggcgaatgattttatcccccgtaaagtcgcgcaaagctaa

SEQ ID NO: 19 - DNA, 1089 bp, Escherichia coli
atgcctcacaatcctatccgcgtggtcgtcggcccggctaactacttttcacatccaggaagtttcaatcacctgcacgattttttcactgatgaacaactttctcgcgcggtgtggatctacggcaaacgcgccattgctgcggcgcaaaccaaacttccgccagcgtttggactgccaggggcaaagcatattttgtttcgcggtcattgcagcgaaagcgatgtacaacaactggcggctgagtccggtgacgaccgcagcgtggtgattggcgtcggtggcggtgcactgctcgacaccgcgaaagccctcgcccgccgtctcggtctgccgtttgttgccgttccgacgatcgccgccacctgcgccgcctggacaccgctctccgtctggtataatgatgccggacaggcgctgcattatgagattttcgacgacgccaattttatggtgctggtggaaccggagattatcctcaatgcaccgcaacaatatctgctggcggggatcggtgacacgctggcgaaatggtatgaagcggtggtgctggctccgcaaccagaaacgttgccgctaaccgtgcgactggggatcaataatgcgcaagccattcgcgacgtcttgttaaacagtagcgaacaggcgctgagcgatcagcaaaatcaacagttaacgcaatcattttgcgatgtggtggatgctattattgctggtggtgggatggttggtggtctgggcgatcgttttacgcgtgtggcggcagctcatgccgtgcataacggtctgaccgtgctgccgcaaaccgagaagtttctccacggcaccaaagtcgcctacggaattctggtgcaaagcgccttgctgggtcaggatgatgtgctggcgcaattaactggagcgtatcagcgttttcatctgccgactacactggcggagctggaagtggatatcaataatcaggcggagatcgacaaagtgattgcccacaccctgcgtccggtggagtccattcattacctgccagtcacgctgacaccagatacgttgcgtgcagcgttcaaaaaagtggaatcgtttaaagcctga

SEQ ID NO: 20 - DNA, 1425 bp, Escherichia coli
atgcaacataagttactgattaacggagaactggttagcggcgaaggggaaaaacagcctgtctataatccggcaacgggggacgttttactggaaattgccgaggcatccgcagagcaggtcgatgctgctgtgcgcgcggcagatgcagcatttgccgaatgggggcaaaccacgccgaaagtgcgtgcggaatgtctgctgaaactggctgatgttatcgaagaaaatggtcaggtttttgccgaactggagtcccgtaattgtggcaaaccgctgcatagtgcgttcaatgatgaaatcccggcgattgtcgatgtttttcgctttttcgcgggtgcggcgcgctgtctgaatggtctggcggcaggtgaatatcttgaaggtcatacttcgatgatccgtcgcgatccgttgggggtcgtggcttctatcgcaccgtggaattatccgctgatgatggccgcgtggaaacttgctccggcgctggcggcagggaactgcgtagtgcttaaaccatcagaaattaccccgctgaccgcgttgaagttggcagagctggcgaaagatatcttcccggcaggcgtgattaacatactgtttggcagaggcaaaacggtgggtgatccgctgaccggtcatcccaaagtgcggatggtgtcgctgacgggctctatcgccaccggcgagcacatcatcagccataccgcgtcgtccattaagcgtactcatatggaacttggtggcaaagcgccagtgattgtttttgatgatgcggatattgaagcagtggtcgaaggtgtacgtacatttggctattacaatgctggacaggattgtactgcggcttgtcggatctacgcgcaaaaaggcatttacgatacgctggtggaaaaactgggtgctgcggtggcaacgttaaaatctggtgcgccagatgacgagtctacggagcttggacctttaagctcgctggcgcatctcgaacgcgtcggcaaggcagtagaagaggcgaaagcgacagggcacatcaaagtgatcactggcggtgaaaagcgcaagggtaatggctattactatgcgccgacgctgctggctggcgcattacaggacgatgccatcgtgcaaaaagaggtatttggtccagtagtgagtgttacgcccttcgacaacgaagaacaggtggtgaactgggcgaatgacagccagtacggacttgcatcttcggtatggacgaaagatgtgggcagggcgcatcgcgtcagcgcacggctgcaatatggttgtacctgggtcaatacccatttcatgctggtaagtgaaatgccgcacggtgggcagaaactttctggttacggcaaggatatgtcactttatgggctggaggattacaccgtcgtccgccacgtcatggttaaacattaa

SEQ ID NO: 21 - DNA, 909 bp, Escherichia coli
atgaaaacgggatctgagtttcatgtcggtatcgttggcttagggtcaatgggaatgggagcagcactgtcatatgtccgcgcaggtctttctacctggggcgcagacctgaacagcaatgcctgcgctacgttgaaagaggcaggtgcttgcggggtttctgataacgccgcgacgtttgccgaaaaactggacgcactgctggtgctggtggtcaatgcggcccaggttaaacaggtgctgtttggtgaaacaggcgttgcacaacatctgaaacccggtacggcagtaatggtttcttccactatcgctagtgctgatgcgcaagaaattgctaccgctctggctggattcgatctggaaatgctggatgcgccagtttctggtggtgcagtaaaagccgctaacggtgaaatgactgtcatggcctccggtagcgatattgcctttgaacgactggcacccgtgctggaagccgttgccggaaaagtttatcgcataggtgcagaaccgggactaggttcgaccgtaaaaattattcaccagttgttagcgggcgtacatattgctgccggagccgaagcgatggcacttgcagcccgtgcggggatcccgctggatgtgatgtatgacgtcgtgaccaatgccgccggaaattcctggatgttcgaaaaccggatgcgtcatgtggtggatggcgattacaccccgcattcagccgtcgatatttttgttaaggatcttggtctggttgccgatacagccaaagccctgcacttcccgctgccattggcctcaacagcattgaatatgttcaccagcgccagtaacgcgggttacgggaaagaagacgatagcgcagttatcaagattttctctggcatcactctaccgggagcgaaatcatga

SEQ ID NO: 22 - DNA, 1152 bp, Escherichia coli
atggcagcttcaacgttctttattccttctgtgaatgtcatcggcgctgattcattgactgatgcaatgaatatgatggcagattatggatttacccgtaccttaattgtcactgacaatatgttaacgaaattaggtatggcgggcgatgtgcaaaaagcactggaagaacgcaatatttttagcgttatttatgatggcacccaacctaaccccaccacggaaaacgtcgccgcaggtttgaaattacttaaagagaataattgcgatagcgtgatctccttaggcggtggttctccacacgactgcgcaaaaggtattgcgctggtggcagccaatggcggcgatattcgcgattacgaaggcgttgaccgctctgcaaaaccgcagctgccgatgatcgccatcaataccacggcgggtacggcctctgaaatgacccgtttctgcatcatcactgacgaagcgcgtcatatcaaaatggcgattgttgataaacatgtcactccgctgctttctgtcaatgactcctctctgatgattggtatgccgaagtcactgaccgccgcaacgggtatggatgccttaacgcacgctatcgaagcatatgtttctattgccgccacgccgatcactgacgcttgtgcactgaaagccgtgaccatgattgccgaaaacctgccgttagccgttgaagatggcagtaatgcgaaagcgcgtgaagcaatggcttatgcccagttcctcgccggtatggcgttcaataatgcttctctgggttatgttcatgcgatggcgcaccagctgggcggtttctacaacctgccacacggtgtatgtaacgccgttttgctgccgcacgttcaggtattcaacagcaaagtcgccgctgcacgtctgcgtgactgtgccgctgcaatgggcgtgaacgtgacaggtaaaaacgacgcggaaggtgctgaagcctgcattaacgccatccgtgaactggcgaagaaagtggatatcccggcaggcctacgcgacctgaacgtgaaagaagaagatttcgcggtattggcgactaatgccctgaaagatgcctgtggctttactaacccgatccaggcaactcacgaagaaattgtggcgatttatcgcgcagcgatgtaa

SEQ ID NO: 23 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 24 - protein, 512 aa, Escherichia coli
MTNNPPSAQIKPGEYGFPLKLKARYDNFIGGEWVAPADGEYYQNLTPVTGQLLCEVASSGKRDIDLALDAAHKVKDKWAHTSVQDRAAILFKIADRMEQNLELLATAETWDNGKPIRETSAADVPLAIDHFRYFASCIRAQEGGISEVDSETVAYHFHEPLGVVGQIIPWNFPLLMASWKMAPALAAGNCVVLKPARLTPLSVLLLMEIVGDLLPPGVVNVVNGAGGVIGEYLATSKRIAKVAFTGSTEVGQQIMQYATQNIIPVTLELGGKSPNIFFADVMDEEDAFFDKALEGFALFAFNQGEVCTCPSRALVQESIYERFMERAIRRVESIRSGNPLDSVTQMGAQVSHGQLETILNYIDIGKKEGADVLTGGRRKLLEGELKDGYYLEPTILFGQNNMRVFQEEIFGPVLAVTTFKTMEEALELANDTQYGLGAGVWSRNGNLAYKMGRGIQAGRVWTNCYHAYPAHAAFGGYKQSGIGRETHKMMLEHYQQTKCLLVSYSDKPLGLF

SEQ ID NO: 25 - protein, 490 aa, Escherichia coli
MSRMAEQQLYIHGGYTSATSGRTFETINPANGNVLATVQAAGREDVDRAVKSAQQGQKIWASMTAMERSRILRRAVDILRERNDELAKLETLDTGKAYSETSTVDIVTGADVLEYYAGLIPALEGSQIPLRETSFVYTRREPLGVVAGIGAWNYPIQIALWKSAPALAAGNAMIFKPSEVTPLTALKLAEIYSEAGLPDGVFNVLPGVGAETGQYLTEHPGIAKVSFTGGVASGKKVMANSAASSLKEVTMELGGKSPLIVFDDADLDLAADIAMMANFFSSGQVCTNGTRVFVPAKCKAAFEQKILARVERIRAGDVFDPQTNFGPLVSFPHRDNVLRYIAKGKEEGARVLCGGDVLKGDGFDNGAWVAPTVFTDCSDDMTIVREEIFGPVMSILTYESEDEVIRRANDTDYGLAAGIVTADLNRAHRVIHQLEAGICWINTWGESPAEMPVGGYKHSGIGRENGVMTLQSYTQVKSIQVEMAKFQSIF

SEQ ID NO: 26 - protein, 467 aa, Escherichia coli
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 27 - protein, 395 aa, Escherichia coli
MQNELQTALFQAFDTLNLQRVKTFSVPPVTLCGPGSVSSCGQQAQTRGLKHLFVMADSFLHQAGMTAGLTRSLTVKGIAMTLWPCPVGEPCITDVCAAVAQLRESGCDGVIAFGGGSVLDAAKAVTLLVTNPDSTLAEMSETSVLQPRLPLIAIPTTAGTGSETTNVTVIIDAVSGRKQVLAHASLMPDVAILDAALTEGVPSHVTAMTGIDALTHAIEAYSALNATPFTDSLAIGAIAMIGKSLPKAVGYGHDLAARESMLLASCMAGMAFSSAGLGLCHAMAHQPGAALHIPHGLANAMLLPTVMEFNRMVCRERFSQIGRALRTKKSDDRDAINAVSELIAEVGIGKRLGDVGATSAHYGAWAQAALEDICLRSNPRTASLEQIVGLYAAAQ

SEQ ID NO: 28 - protein, 383 aa, Escherichia coli
MMANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 29 - protein, 482 aa, Escherichia coli
MKLNDSNLFRQQALINGEWLDANNGEAIDVTNPANGDKLGSVPKMGADETRAAIDAANRALPAWRALTAKERATILRNWFNLMMEHQDDLARLMTLEQGKPLAEAKGEISYAASFIEWFAEEGKRIYGDTIPGHQADKRLIVIKQPIGVTAAITPWNFPAAMITRKAGPALAAGCTMVLKPASQTPFSALALAELAIRAGVPAGVFNVVTGSAGAVGNELTSNPLVRKLSFTGSTEIGRQLMEQCAKDIKKVSLELGGNAPFIVFDDADLDKAVEGALASKFRNAGQTCVCANRLYVQDGVYDRFAEKLQQAVSKLHIGDGLDNGVTIGPLIDEKAVAKVEEHIADALEKGARVVCGGKAHERGGNFFQPTILVDVPANAKVSKEETFGPLAPLFRFKDEADVIAQANDTEFGLAAYFYARDLSRVFRVGEALEYGIVGINTGIISNEVAPFGGIKASGLGREGSKYGIEDYLEIKYMCIGL

SEQ ID NO: 30 - protein, 296 aa, Escherichia coli
MTMKVGFIGLGIMGKPMSKNLLKAGYSLVVADRNPEAIADVIAAGAETASTAKAIAEQCDVIITMLPNSPHVKEVALGENGIIEGAKPGTVLIDMSSIAPLASREISEALKAKGIDMLDAPVSGGEPKAIDGTLSVMVGGDKAIFDKYYDLMKAMAGSVVHTGEIGAGNVTKLANQVIVALNIAAMSEALTLATKAGVNPDLVYQAIRGGLAGSTVLDAKAPMVMDRNFKPGFRIDLHIKDLANALDTSHGVGAQLPLTAAVMEMMQALRADGLGTADHSALACYYEKLAKVEVTR

SEQ ID NO: 31 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 32 - protein, 292 aa, Escherichia coli
MKLGFIGLGIMGTPMAINLARAGHQLHVTTIGPVADELLSLGAVSVETARQVTEASDIIFIMVPDTPQVEEVLFGENGCTKASLKGKTIVDMSSISPIETKRFARQVNELGGDYLDAPVSGGEIGAREGTLSIMVGGDEAVFERVKPLFELLGKNITLVGGNGDGQTCKVANQIIVALNIEAVSEALLFASKAGADPVRVRQALMGGFASSRILEVHGERMIKRTFNPGFKIALHQKDLNLALQSAKALALNLPNTATCQELFNTCAANGGSQLDHSALVQALELMANHKLA

SEQ ID NO: 33 - protein, 468 aa, Escherichia coli
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 34 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 35 - protein, 681 aa, Escherichia coli
MQQLASFLSGTWQSGRGRSRLIHHAISGEALWEVTSEGLDMAAARQFAIEKGAPALRAMTFIERAAMLKAVAKHLLSEKERFYALSAQTGATRADSWVDIEGGIGTLFTYASLGSRELPDDTLWPEDELIPLSKEGGFAARHLLTSKSGVAVHINAFNFPCWGMLEKLAPTWLGGMPAIIKPATATAQLTQAMVKSIVDSGLVPEGAISLICGSAGDLLDHLDSQDVVTFTGSAATGQMLRVQPNIVAKSIPFTMEADSLNCCVLGEDVTPDQPEFALFIREVVREMTTKAGQKCTAIRRIIVPQALVNAVSDALVARLQKVVVGDPAQEGVKMGALVNAEQRADVQEKVNILLAAGCEIRLGGQADLSAAGAFFPPTLLYCPQPDETPAVHATEAFGPVATLMPAQNQRHALQLACAGGGSLAGTLVTADPQIARQFIADAARTHGRIQILNEESAKESTGHGSPLPQLVHGGPGRAGGGEELGGLRAVKHYMQRTAVQGSPTMLAAISKQWVRGAKVEEDRIHPFRKYFEELQPGDSLLTPRRTMTEADIVNFACLSGDHFYAHMDKIAAAESIFGERVVHGYFVLSAAAGLFVDAGVGPVIANYGLESLRFIEPVKPGDTIQVRLTCKRKTLKKQRSAEEKPTGVVEWAVEVFNQHQTPVALYSILTLVARQHGDFVD

SEQ ID NO: 36 - protein, 417 aa, Escherichia coli
MLEQMGIAAKQASYKLAQLSSREKNRVLEKIADELEAQSEIILNANAQDVADARANGLSEAMLDRLALTPARLKGIADDVRQVCNLADPVGQVIDGGVLDSGLRLERRRVPLGVIGVIYEARPNVTVDVASLCLKTGNAVILRGGKETCRTNAATVAVIQDALKSCGLPAGAVQAIDNPDRALVSEMLRMDKYIDMLIPRGGAGLHKLCREQSTIPVITGGIGVCHIYVDESVEIAEALKVIVNAKTQRPSTCNTVETLLVNKNIADSFLPALSKQMAESGVTLHADAAALAQLQAGPAKVVAVKAEEYDDEFLSLDLNVKIVSDLDDAIAHIREHGTQHSDAILTRDMRNAQRFVNEVDSSAVYVNASTRFTDGGQFGLGAEVAVSTQKLHARGPMGLEALTTYKWIGIGDYTIRA

SEQ ID NO: 37 - protein, 1320 aa, Escherichia coli
MGTTTMGVKLDDATRERIKSAATRIDRTPHWLIKQAIFSYLEQLENSDTLPELPALLSGAANESDEAPTPAEEPHQPFLDFAEQILPQSVSRAAITAAYRRPETEAVSMLLEQARLPQPVAEQAHKLAYQLADKLRNQKNASGRAGMVQGLLQEFSLSSQEGVALMCLAEALLRIPDKATRDALIRDKISNGNWQSHIGRSPSLFVNAATWGLLFTGKLVSTHNEASLSRSLNRIIGKSGEPLIRKGVDMAMRLMGEQFVTGETIAEALANARKLEEKGFRYSYDMLGEAALTAADAQAYMVSYQQAIHAIGKASNGRGIYEGPGISIKLSALHPRYSRAQYDRVMEELYPRLKSLTLLARQYDIGINIDAEESDRLEISLDLLEKLCFEPELAGWNGIGFVIQAYQKRCPLVIDYLIDLATRSRRRLMIRLVKGAYWDSEIKRAQMDGLEGYPVYTRKVYTDVSYLACAKKLLAVPNLIYPQFATHNAHTLAAIYQLAGQNYYPGQYEFQCLHGMGEPLYEQVTGKVADGKLNRPCRIYAPVGTHETLLAYLVRRLLENGANTSFVNRIADTSLPLDELVADPVTAVEKLAQQEGQTGLPHPKIPLPRDLYGHGRDNSAGLDLANEHRLASLSSALLNSALQKWQALPMLEQPVAAGEMSPVINPAEPKDIVGYVREATPREVEQALESAVNNAPIWFATPPAERAAILHRAAVLMESQMQQLIGILVREAGKTFSNAIAEVREAVDFLHYYAGQVRDDFANETHRPLGPVVCISPWNFPLAIFTGQIAAALAAGNSVLAKPAEQTPLIAAQGIAILLEAGVPPGVVQLLPGRGETVGAQLTGDDRVRGVMFTGSTEVATLLQRNIASRLDAQGRPIPLIAETGGMNAMIVDSSALTEQVVVDVLASAFDSAGQRCSALRVLCLQDEIADHTLKMLRGAMAECRMGNPGRLTTDIGPVIDSEAKANIERHIQTMRSKGRPVFQAVRENSEDAREWQSGTFVAPTLIELDDFAELQKEVFGPVLHVVRYNRNQLPELIEQINASGYGLTLGVHTRIDETIAQVTGSAHVGNLYVNRNMVGAVVGVQPFGGEGLSGTGPKAGGPLYLYRLLANRPESALAVTLARQDAKYPVDAQLKAALTQPLNALREWAANRPELQALCTQYGELAQAGTQRLLPGPTGERNTWTLLPRERVLCIADDEQDALTQLAAVLAVGSQVLWPDDALHRQLVKALPSAVSERIQLAKAENITAQPFDAVIFHGDSDQLRALCEAVAARDGTIVSVQGFARGESNILLERLYIERSLSVNTAAAGGNASLMTIG

SEQ ID NO: 38 - protein, 495 aa, Escherichia coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 39 - protein, 462 aa, Escherichia coli
MTITPATHAISINPATGEQLSVLPWAGADDIENALQLAAAGFRDWRETNIDYRAEKLRDIGKALRARSEEMAQMITREMGKPINQARAEVAKSANLCDWYAEHGPAMLKAEPTLVENQQAVIEYRPLGTILAIMPWNFPLWQVMRGAVPIILAGNGYLLKHAPNVMGCAQLIAQVFKDAGIPQGVYGWLNADNDGVSQMIKDSRIAAVTVTGSVRAGAAIGAQAGAALKKCVLELGGSDPFIVLNDADLELAVKAAVAGRYQNTGQVCAAAKRFIIEEGIASAFTERFVAAAAALKMGDPRDEENALGPMARFDLRDELHHQVEKTLAQGARLLLGGEKMAGAGNYYPPTVLANVTPEMTAFREEMFGPVAAITIAKDAEHALELANDSEFGLSATIFTTDETQARQMAARLECGGVFINGYCASDARVAFGGVKKSGFGRELSHFGLHEFCNIQTVWKDRI

SEQ ID NO: 40 - protein, 381 aa, Escherichia coli
MSLNMFWFLPTHGDGHYLGTEEGSRPVDHGYLQQIAQAADRLGYTGVLIPTGRSCEDAWLVAASMIPVTQRLKFLVALRPSVTSPTVAARQAATLDRLSNGRALFNLVTGSDPQELAGDGVFLDHSERYEASAEFTQVWRRLLQRETVDFNGKHIHVRGAKLLFPAIQQPYPPLYFGGSSDVAQELAAEQVDLYLTWGEPPELVKEKIEQVRAKAAAHGRKIRFGIRLHVIVRETNDEAWQAAERLISHLDDETIAKAQAAFARTDSVGQQRMAALHNGKRDNLEISPNLWAGVGLVRGGAGTALVGDGPTVAARINEYAALGIDSFVLSGYPHLEEAYRVGELLFPLLDVAIPEIPQPQPLNPQGEAVANDFIPRKVAQS

SEQ ID NO: 41 - protein, 362 aa, Escherichia coli
MPHNPIRVVVGPANYFSHPGSFNHLHDFFTDEQLSRAVWIYGKRAIAAAQTKLPPAFGLPGAKHILFRGHCSESDVQQLAAESGDDRSVVIGVGGGALLDTAKALARRLGLPFVAVPTIAATCAAWTPLSVWYNDAGQALHYEIFDDANFMVLVEPEIILNAPQQYLLAGIGDTLAKWYEAVVLAPQPETLPLTVRLGINNAQAIRDVLLNSSEQALSDQQNQQLTQSFCDVVDAIIAGGGMVGGLGDRFTRVAAAHAVHNGLTVLPQTEKFLHGTKVAYGILVQSALLGQDDVLAQLTGAYQRFHLPTTLAELEVDINNQAEIDKVIAHTLRPVESIHYLPVTLTPDTLRAAFKKVESFKA

SEQ ID NO: 42 - protein, 474 aa, Escherichia coli
MQHKLLINGELVSGEGEKQPVYNPATGDVLLEIAEASAEQVDAAVRAADAAFAEWGQTTPKVRAECLLKLADVIEENGQVFAELESRNCGKPLHSAFNDEIPAIVDVFRFFAGAARCLNGLAAGEYLEGHTSMIRRDPLGVVASIAPWNYPLMMAAWKLAPALAAGNCVVLKPSEITPLTALKLAELAKDIFPAGVINILFGRGKTVGDPLTGHPKVRMVSLTGSIATGEHIISHTASSIKRTHMELGGKAPVIVFDDADIEAVVEGVRTFGYYNAGQDCTAACRIYAQKGIYDTLVEKLGAAVATLKSGAPDDESTELGPLSSLAHLERVGKAVEEAKATGHIKVITGGEKRKGNGYYYAPTLLAGALQDDAIVQKEVFGPVVSVTPFDNEEQVVNWANDSQYGLASSVWTKDVGRAHRVSARLQYGCTWVNTHFMLVSEMPHGGQKLSGYGKDMSLYGLEDYTVVRHVMVKH

SEQ ID NO: 43 - protein, 302 aa, Escherichia coli
MKTGSEFHVGIVGLGSMGMGAALSYVRAGLSTWGADLNSNACATLKEAGACGVSDNAATFAEKLDALLVLVVNAAQVKQVLFGETGVAQHLKPGTAVMVSSTIASADAQEIATALAGFDLEMLDAPVSGGAVKAANGEMTVMASGSDIAFERLAPVLEAVAGKVYRIGAEPGLGSTVKIIHQLLAGVHIAAGAEAMALAARAGIPLDVMYDVVTNAAGNSWMFENRMRHVVDGDYTPHSAVDIFVKDLGLVADTAKALHFPLPLASTALNMFTSASNAGYGKEDDSAVIKIFSGITLPGAKS

SEQ ID NO: 44 - protein, 383 aa, Escherichia coli
MAASTFFIPSVNVIGADSLTDAMNMMADYGFTRTLIVTDNMLTKLGMAGDVQKALEERNIFSVIYDGTQPNPTTENVAAGLKLLKENNCDSVISLGGGSPHDCAKGIALVAANGGDIRDYEGVDRSAKPQLPMIAINTTAGTASEMTRFCIITDEARHIKMAIVDKHVTPLLSVNDSSLMIGMPKSLTAATGMDALTHAIEAYVSIAATPITDACALKAVTMIAENLPLAVEDGSNAKAREAMAYAQFLAGMAFNNASLGYVHAMAHQLGGFYNLPHGVCNAVLLPHVQVFNSKVAAARLRDCAAAMGVNVTGKNDAEGAEACINAIRELAKKVDIPAGLRDLNVKEEDFAVLATNALKDACGFTNPIQATHEEIVAIYRAAM

SEQ ID NO: 45 - DNA, 20 bp, artificial sequence
  chemically synthesized
atggctgttactaatgtcgc

SEQ ID NO: 46 - DNA, 24 bp, artificial sequence
  chemically synthesized
agcggattttttcgcttttttctc

SEQ ID NO: 47 - DNA, 20 bp, artificial sequence
  chemically synthesized
atgaaggctgcagttgttac

SEQ ID NO: 48 - DNA, 19 bp, artificial sequence
  chemically synthesized
gtgacggaaatcaatcacc

SEQ ID NO: 49 - DNA, 19 bp, artificial sequence
  chemically synthesized
atgtcagtacccgttcaac

SEQ ID NO: 50 - DNA, 22 bp, artificial sequence
  chemically synthesized
agactgtaaataaaccacctgg

SEQ ID NO: 51 - DNA, 21 bp, artificial sequence
  chemically synthesized
atgaccaataatcccccttca

SEQ ID NO: 52 - DNA, 14 bp, artificial sequence
  chemically synthesized
gaacagccccaacg

SEQ ID NO: 53 - DNA, 24 bp, artificial sequence
  chemically synthesized
atgactttatggattaacggtgac

SEQ ID NO: 54 - DNA, 15 bp, artificial sequence
  chemically synthesized
tcgcaccacctcatc

SEQ ID NO: 55 - DNA, 19 bp, artificial sequence
  chemically synthesized
atgtcccgaatggcagaac

SEQ ID NO: 56 - DNA, 22 bp, artificial sequence
  chemically synthesized
gaatatggactggaatttagcc

SEQ ID NO: 57 - DNA, 25 bp, artificial sequence
  chemically synthesized
atggctaatccaaccgttattaagc

SEQ ID NO: 58 - DNA, 15 bp, artificial sequence
  chemically synthesized
gccgccgaactggtc

SEQ ID NO: 59 - DNA, 20 bp, artificial sequence
  chemically synthesized
atggctatccctgcatttgg

SEQ ID NO: 60 - DNA, 19 bp, artificial sequence
  chemically synthesized
atcccattcaggagccaga

SEQ ID NO: 61 - DNA, 24 bp, artificial sequence
  chemically synthesized
atgaatcaacaggatattgaacag

SEQ ID NO: 62 - DNA, 19 bp, artificial sequence
  chemically synthesized
aacaatgcgaaacgcatcg

SEQ ID NO: 63 - DNA, 22 bp, artificial sequence
  chemically synthesized
atgcaaaatgaattgcagaccg

SEQ ID NO: 64 - DNA, 15 bp, artificial sequence
  chemically synthesized
ttgcgccgctgcgta

SEQ ID NO: 65 - DNA, 18 bp, artificial sequence
  chemically synthesized
atgacagagccgcatgta

SEQ ID NO: 66 - DNA, 19 bp, artificial sequence
  chemically synthesized
ataccgtacacacaccgac

SEQ ID NO: 67 - DNA, 24 bp, artificial sequence
  chemically synthesized
atgatggctaacagaatgattctg

SEQ ID NO: 68 - DNA, 18 bp, artificial sequence
  chemically synthesized
ccaggcggtatggtaaag

SEQ ID NO: 69 - DNA, 25 bp, artificial sequence
  chemically synthezised
atgaaacttaacgacagtaacttat

SEQ ID NO: 70 - DNA, 19 bp, artificial sequence
  chemically synthesized
aagaccgatgcacatatat

SEQ ID NO: 71 - DNA, 25 bp, artificial sequence
  chemically synthesized
atgactatgaaagttggttttattg

SEQ ID NO: 72 - DNA, 19 bp, artificial sequence
  chemically synthesized
acgagtaacttcgactttc

SEQ ID NO: 73 - DNA, 20 bp, artificial sequence
  chemically synthesized
atggaccgcattattcaatc

SEQ ID NO: 74 - DNA, 20 bp, artificial sequence
  chemically synthesized
ttcccactcttgcaggaaac

SEQ ID NO: 75 - DNA, 25 bp, artificial sequence
  chemically synthesized
atgaaactgggatttattggcttag

SEQ ID NO: 76 - DNA, 19 bp, artificial sequence
  chemically synthesized
ggccagtttatggttagcc

SEQ ID NO: 77 - DNA, 20 bp, artificial sequence
  chemically synthesized
atgtccaagcaacagatcgg

SEQ ID NO: 78 - DNA, 19 bp, artificial sequence
  chemically synthesized
atccagccattcggtatgg

SEQ ID NO: 79 - DNA, 21 bp, artificial sequence
  chemically synthesized
atgaaactcgccgtttatagc

SEQ ID NO: 80 - DNA, 17 bp, artificial sequence
  chemically synthesized
aaccagttcgttcgggc

SEQ ID NO: 81 - DNA, 21 bp, artificial sequence
  chemically synthesized
atgcagcagttagccagtttc

SEQ ID NO: 82 - DNA, 21 bp, artificial sequence
  chemically synthesized
atcgacaaaatcaccgtgctg

SEQ ID NO: 83 - DNA, 20 bp, artificial sequence
  chemically synthesized
atgctggaacaaatgggcat

SEQ ID NO: 84 - DNA, 18 bp, artificial sequence
  chemically synthesized
cgcacgaatggtgtaatc

SEQ ID NO: 85 - DNA, 18 bp, artificial sequence
  chemically synthesized
atgggaaccaccaccatg

SEQ ID NO: 86 - DNA, 22 bp, artificial sequence
  chemically synthesized
acctatagtcattaagctggcg

SEQ ID NO: 87 - DNA, 24 bp, artificial sequence
  chemically synthesized
atgaattttcatcatctggcttac

SEQ ID NO: 88 - DNA, 17 bp, artificial sequence
  chemically synthesized
ggcctccaggcttatcc

SEQ ID NO: 89 - DNA, 20 bp, artificial sequence
  chemically synthesized
atgaccattactccggcaac

SEQ ID NO: 90 - DNA, 19 bp, artificial sequence
  cheically synthesized
agatccggtctttccacac

SEQ ID NO: 91 - DNA, 24 bp, artificial sequence
  chemically synthesized
atgattagtctattcgacatgtta

SEQ ID NO: 92 - DNA, 20 bp, artificial sequence
  chemically synthesized
gtcacactggactttgattg

SEQ ID NO: 93 - DNA, 24 bp, artificial sequence
  chemically synthesized
atgattagcgtattcgatattttc

SEQ ID NO: 94 - DNA, 19 bp, artificial sequence
  chemically synthesized
atcgcaggcaacgatcttc

SEQ ID NO: 95 - DNA, 23 bp, artificial sequenceq
  chemically synthesized
atgagtctgaatatgttctggtt

SEQ ID NO: 96 - DNA, 18 bp, artificial sequence
  chemically synthesized
gctttgcgcgactttacg

SEQ ID NO: 97 - DNA, 22 bp, artificial sequence
  chemically synthesized
atgcatattacatacgatctgc

SEQ ID NO: 98 - DNA, 18 bp, artificial sequence
  chemically synthesized
agcgtcaacgaaaccggt

SEQ ID NO: 99 - DNA, 24 bp, artificial sequence
  chemically synthesized
atgattagtgcattcgatattttc

SEQ ID NO: 100 - DNA, 18 bp, artificial sequence
  chemically synthesized
gccgcagaccactttaat

SEQ ID NO: 101 - DNA, 20 bp, artificial sequence
  chemically synthsized
atgtctgaaggctggaacat

SEQ ID NO: 102 - DNA, 19 bp, artificial sequence
  chemically synthesized
gtacagatactcctgcacc

SEQ ID NO: 103 - DNA, 20 bp, artificial sequence
  chemically synthesized
atgcctcacaatcctatccg

SEQ ID NO: 104 - DNA, 20 bp, artificial sequence
  chemically synthesized
ggctttaaacgattccactt

SEQ ID NO: 105 - DNA, 25 bp, artificial sequence
  chemically synthesized
atgcaacataagttactgattaacg

SEQ ID NO: 106 - DNA, 20 bp, artificial sequence
  chemically synthesized
tacaaattggtactgcaccg

SEQ ID NO: 107 - DNA, 26 bp, artificial sequence
  chemically synthesized
atgcaacaaaaaatgattcaatttag

SEQ ID NO: 108 - DNA, 19 bp, artificial sequence
  chemically synthesized
caccatatccagcgcagtt

SEQ ID NO: 109 - DNA, 22 bp, artificial sequence
  chemically synthesized
atgaaaacgggatctgagtttc

SEQ ID NO: 110 - DNA, 18 bp, artificial sequence
  chemically synthesized
tgatttcgctcccggtag

SEQ ID NO: 111 - DNA, 24 bp, artificial sequence
  chemically synthesized
atgttacgcgataaatttattcac

SEQ ID NO: 112 - DNA, 18 bp, artificial sequence
  chemically synthesized
cccccgtccaaactccag

SEQ ID NO: 113 - DNA, 20 bp, artificial sequence
  chemically synthesized
atggtctggttagcgaatcc

SEQ ID NO: 114 - DNA, 19 bp, artificial sequence
  chemically synthesized
tttatcggaagacgcctgc

SEQ ID NO: 115 - DNA, 20 bp, artificial sequence
  chemically synthesized
atggcagcttcaacgttctt

SEQ ID NO: 116 - DNA, 19 bp, artificial seuence
  chemically synthesized
catcgctgcgcgataaatc

SEQ ID NO: 117 - DNA, 23 bp, artificial sequence
  chemically synthesized
atgaacaactttaatctgcacac

SEQ ID NO: 118 - DNA, 19 bp, artificial sequence
  chemicallyl synthesized
gcgggcggcttcgtatata

SEQ ID NO: 119 - DNA, 4381 bp, artificial sequence
  chemically synthesized
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggcccttaagggcgaattcgaagcttacgtagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 120 - DNA, 1014 bp, Escherichia coli
atgtctgaaggctggaacattgccgtcctgggcgcaactggcgctgtgggcgaagccctgcttgaaacgctggctgaacgtcagttcccggttggggaaatttatgcactggcacgtaacgaaagcgcaggcgaacaactgcgctttggtggtaagacaatcaccgtgcaggatgccgctgaattcgactggacgcaggcgcagctggcattttttgtcgcaggcaaagaagctaccgctgcctgggttgaagaagcgaccaactcaggttgcctggtgatcgacagcagtggattgtttgctctcgaacccgacgtaccgctggtggtgccggaagtaaacccgtttgtactgacagattaccggaaccggaatgtcatcgccgtaccagacagtctgaccagccagctgctggcggcactgaaaccgttaatcgatcagggcggtttatcacgtatcagcgttaccagcctgatttcagcctccgcccagggcaaaaaagcggtcgatgcgttagcggggcagagtgcgaaattgctcaacggcattccgattgacgaagaagatttcttcgggcgtcagctggcgttcaacatgctgccgttactgccggatagcgaaggtagcgtgcgtgaagaacgtcgtatcgttgacgaagtacgcaaaatcctgcaggacgaagggctgatgatttcggctagcgtcgtccaggcaccggtattctacggtcatgcccagatggtcaactttgaagctctgcgtccactggcagcagaagaagcgcgtgatgcgtttgttcaaggcgaagatattgtgctctctgaagagaacgaattcccaactcaggtaggtgatgcttcgggtacgccgcatctttctgttggctgcgtgcgtaatgactacggtatgccggagcaagtccagttctggtcggtggccgataacgttcgctttggcggcgcgctgatggcagtaaaaatcgccgagaaactggtgcaggagtatctgtactaa

SEQ ID NO: 121 - protein, 337 aa, Escherichia coli
MSEGWNIAVLGATGAVGEALLETLAERQFPVGEIYALARNESAGEQLRFGGKTITVQDAAEFDWTQAQLAFFVAGKEATAAWVEEATNSGCLVIDSSGLFALEPDVPLVVPEVNPFVLTDYRNRNVIAVPDSLTSQLLAALKPLIDQGGLSRISVTSLISASAQGKKAVDALAGQSAKLLNGIPIDEEDFFGRQLAFNMLPLLPDSEGSVREERRIVDEVRKILQDEGLMISASVVQAPVFYGHAQMVNFEALRPLAAEEARDAFVQGEDIVLSEENEFPTQVGDASGTPHLSVGCVRNDYGMPEQVQFWSVADNVRFGGALMAVKIAEKLVQEYLY

SEQ ID NO: 122 - protein, 1232 aa, Chloroflexus aurantiacus
MRVKFHTTGETIMAGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 123 - DNA, 8252 bp, artificial sequence
  chemically synthesized
gaattccgctagcaggagctaaggaagctaaaatgtccggtacgggtcgtttggctggtaaaattgcattgatcaccggtggtgctggtaacattggttccgagctgacccgccgttttctggccgagggtgcgacggttattatcagcggccgtaaccgtgcgaagctgaccgcgctggccgagcgcatgcaagccgaggccggcgtgccggccaagcgcattgatttggaggtgatggatggttccgaccctgtggctgtccgtgccggtatcgaggcaatcgtcgctcgccacggtcagattgacattctggttaacaacgcgggctccgccggtgcccaacgtcgcttggcggaaattccgctgacggaggcagaattgggtccgggtgcggaggagactttgcacgcttcgatcgcgaatctgttgggcatgggttggcacctgatgcgtattgcggctccgcacatgccagttggctccgcagttatcaacgtttcgactattttctcgcgcgcagagtactatggtcgcattccgtacgttaccccgaaggcagcgctgaacgctttgtcccagctggctgcccgcgagctgggcgctcgtggcatccgcgttaacactattttcccaggtcctattgagtccgaccgcatccgtaccgtgtttcaacgtatggatcaactgaagggtcgcccggagggcgacaccgcccatcactttttgaacaccatgcgcctgtgccgcgcaaacgaccaaggcgctttggaacgccgctttccgtccgttggcgatgttgctgatgcggctgtgtttctggcttctgctgagagcgcggcactgtcgggtgagacgattgaggtcacccacggtatggaactgccggcgtgtagcgaaacctccttgttggcgcgtaccgatctgcgtaccatcgacgcgagcggtcgcactaccctgatttgcgctggcgatcaaattgaagaagttatggccctgacgggcatgctgcgtacgtgcggtagcgaagtgattatcggcttccgttctgcggctgccctggcgcaatttgagcaggcagtgaatgaatctcgccgtctggcaggtgcggatttcaccccgccgatcgctttgccgttggacccacgtgacccggccaccattgatgcggttttcgattggggcgcaggcgagaatacgggtggcatccatgcggcggtcattctgccggcaacctcccacgaaccggctccgtgcgtgattgaagtcgatgacgaacgcgtcctgaatttcctggccgatgaaattaccggcaccatcgttattgcgagccgtttggcgcgctattggcaatcccaacgcctgaccccgggtgcccgtgcccgcggtccgcgtgttatctttctgagcaacggtgccgatcaaaatggtaatgtttacggtcgtattcaatctgcggcgatcggtcaattgattcgcgtttggcgtcacgaggcggagttggactatcaacgtgcatccgccgcaggcgatcacgttctgccgccggtttgggcgaaccagattgtccgtttcgctaaccgctccctggaaggtctggagttcgcgtgcgcgtggaccgcacagctgctgcacagccaacgtcatattaacgaaattacgctgaacattccagccaatattagcgcgaccacgggcgcacgttccgccagcgtcggctgggccgagtccttgattggtctgcacctgggcaaggtggctctgattaccggtggttcggcgggcatcggtggtcaaatcggtcgtctgctggccttgtctggcgcgcgtgtgatgctggccgctcgcgatcgccataaattggaacagatgcaagccatgattcaaagcgaattggcggaggttggttataccgatgtggaggaccgtgtgcacatcgctccgggttgcgatgtgagcagcgaggcgcagctggcagatctggtggaacgtacgctgtccgcattcggtaccgtggattatttgattaataacgccggtattgcgggcgtggaggagatggtgatcgacatgccggtggaaggctggcgtcacaccctgtttgccaacctgatttcgaattattcgctgatgcgcaagttggcgccgctgatgaagaagcaaggtagcggttacatcctgaacgtttcttcctattttggcggtgagaaggacgcggcgattccttatccgaaccgcgccgactacgccgtctccaaggctggccaacgcgcgatggcggaagtgttcgctcgtttcctgggtccagagattcagatcaatgctattgccccaggtccggttgaaggcgaccgcctgcgtggtaccggtgagcgtccgggcctgtttgctcgtcgcgcccgtctgatcttggagaataaacgcctgaacgaattgcacgcggctttgattgctgcggcccgcaccgatgagcgctcgatgcacgagttggttgaattgttgctgccgaacgacgtggccgcgttggagcagaacccagcggcccctaccgcgctgcgtgagctggcacgccgcttccgtagcgaaggtgatccggcggcaagctcctcgtccgccttgctgaatcgctccatcgctgccaagctgttggctcgcttgcataacggtggctatgtgctgccggcggatatttttgcaaatctgcctaatccgccggacccgttctttacccgtgcgcaaattgaccgcgaagctcgcaaggtgcgtgatggtattatgggtatgctgtatctgcagcgtatgccaaccgagtttgacgtcgctatggcaaccgtgtactatctggccgatcgtaacgtgagcggcgaaactttccatccgtctggtggtttgcgctacgagcgtaccccgaccggtggcgagctgttcggcctgccatcgccggaacgtctggcggagctggttggtagcacggtgtacctgatcggtgaacacctgaccgagcacctgaacctgctggctcgtgcctatttggagcgctacggtgcccgtcaagtggtgatgattgttgagacggaaaccggtgcggaaaccatgcgtcgtctgttgcatgatcacgtcgaggcaggtcgcctgatgactattgtggcaggtgatcagattgaggcagcgattgaccaagcgatcacgcgctatggccgtccgggtccggtggtgtgcactccattccgtccactgccaaccgttccgctggtcggtcgtaaagactccgattggagcaccgttttgagcgaggcggaatttgcggaactgtgtgagcatcagctgacccaccatttccgtgttgctcgtaagatcgccttgtcggatggcgcgtcgctggcgttggttaccccggaaacgactgcgactagcaccacggagcaatttgctctggcgaacttcatcaagaccaccctgcacgcgttcaccgcgaccatcggtgttgagtcggagcgcaccgcgcaacgtattctgattaaccaggttgatctgacgcgccgcgcccgtgcggaagagccgcgtgacccgcacgagcgtcagcaggaattggaacgcttcattgaagccgttctgctggttaccgctccgctgcctcctgaggcagacacgcgctacgcaggccgtattcaccgcggtcgtgcgattaccgtctaatagaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaaca

SEQ ID NO: 124 - DNA, 44 bp, artificial sequence
  chemically synthesized
tcgtaccaaccatggccggtacgggtcgtttggctggtaaaatt

SEQ ID NO: 125 - DNA, 25 bp, artificial sequence
  chemically synthesized
ggattagacggtaatcgcacgaccg

SEQ ID NO: 126 - DNA, 26 bp, artificial sequence
  chemically synthesized
gggaacggcggggaaaaacaaacgtt

SEQ ID NO: 127 - DNA, 30 bp, artificial sequence
  chemically synthesized
ggtccatggtaattctccacgcttataagc

SEQ ID NO: 128 - DNA, 25 bp, artificial sequence
  chemically synthesized
gggaacggcggggaaaaacaaacgt

SEQ ID NO: 129 - DNA, 8286 bp, artificial sequence
  chemically synthesized
atgaccatgattacgccaagcgcgcaattaaccctcactaaagggaacaaaagctgggtaccgggccccccctcgaggtcgacggtatcgataagcttgatatccactgtggaattcgcccttggattagacggtaatcgcacgaccgcggtgaatacggcctgcgtagcgcgtgtctgcctcaggaggcagcggagcggtaaccagcagaacggcttcaatgaagcgttccaattcctgctgacgctcgtgcgggtcacgcggctcttccgcacgggcgcggcgcgtcagatcaacctggttaatcagaatacgttgcgcggtgcgctccgactcaacaccgatggtcgcggtgaacgcgtgcagggtggtcttgatgaagttcgccagagcaaattgctccgtggtgctagtcgcagtcgtttccggggtaaccaacgccagcgacgcgccatccgacaaggcgatcttacgagcaacacggaaatggtgggtcagctgatgctcacacagttccgcaaattccgcctcgctcaaaacggtgctccaatcggagtctttacgaccgaccagcggaacggttggcagtggacggaatggagtgcacaccaccggacccggacggccatagcgcgtgatcgcttggtcaatcgctgcctcaatctgatcacctgccacaatagtcatcaggcgacctgcctcgacgtgatcatgcaacagacgacgcatggtttccgcaccggtttccgtctcaacaatcatcaccacttgacgggcaccgtagcgctccaaataggcacgagccagcaggttcaggtgctcggtcaggtgttcaccgatcaggtacaccgtgctaccaaccagctccgccagacgttccggcgatggcaggccgaacagctcgccaccggtcggggtacgctcgtagcgcaaaccaccagacggatggaaagtttcgccgctcacgttacgatcggccagatagtacacggttgccatagcgacgtcaaactcggttggcatacgctgcagatacagcatacccataataccatcacgcaccttgcgagcttcgcggtcaatttgcgcacgggtaaagaacgggtccggcggattaggcagatttgcaaaaatatccgccggcagcacatagccaccgttatgcaagcgagccaacagcttggcagcgatggagcgattcagcaaggcggacgaggagcttgccgccggatcaccttcgctacggaagcggcgtgccagctcacgcagcgcggtaggggccgctgggttctgctccaacgcggccacgtcgttcggcagcaacaattcaaccaactcgtgcatcgagcgctcatcggtgcgggccgcagcaatcaaagccgcgtgcaattcgttcaggcgtttattctccaagatcagacgggcgcgacgagcaaacaggcccggacgctcaccggtaccacgcaggcggtcgccttcaaccggacctggggcaatagcattgatctgaatctctggacccaggaaacgagcgaacacttccgccatcgcgcgttggccagccttggagacggcgtagtcggcgcggttcggataaggaatcgccgcgtccttctcaccgccaaaataggaagaaacgttcaggatgtaaccgctaccttgcttcttcatcagcggcgccaacttgcgcatcagcgaataattcgaaatcaggttggcaaacagggtgtgacgccagccttccaccggcatgtcgatcaccatctcctccacgcccgcaataccggcgttattaatcaaataatccacggtaccgaatgcggacagcgtacgttccaccagatctgccagctgcgcctcgctgctcacatcgcaacccggagcgatgtgcacacggtcctccacatcggtataaccaacctccgccaattcgctttgaatcatggcttgcatctgttccaatttatggcgatcgcgagcggccagcatcacacgcgcgccagacaaggccagcagacgaccgatttgaccaccgatgcccgccgaaccaccggtaatcagagccaccttgcccaggtgcagaccaatcaaggactcggcccagccgacgctggcggaacgtgcgcccgtggtcgcgctaatattggctggaatgttcagcgtaatttcgttaatatgacgttggctgtgcagcagctgtgcggtccacgcgcacgcgaactccagaccttccagggagcggttagcgaaacggacaatctggttcgcccaaaccggcggcagaacgtgatcgcctgcggcggatgcacgttgatagtccaactccgcctcgtgacgccaaacgcgaatcaattgaccgatcgccgcagattgaatacgaccgtaaacattaccattttgatcggcaccgttgctcagaaagataacacgcggaccgcgggcacgggcacccggggtcaggcgttgggattgccaatagcgcgccaaacggctcgcaataacgatggtgccggtaatttcatcggccaggaaattcaggacgcgttcgtcatcgacttcaatcacgcacggagccggttcgtgggaggttgccggcagaatgaccgccgcatggatgccacccgtattctcgcctgcgccccaatcgaaaaccgcatcaatggtggccgggtcacgtgggtccaacggcaaagcgatcggcggggtgaaatccgcacctgccagacggcgagattcattcactgcctgctcaaattgcgccagggcagccgcagaacggaagccgataatcacttcgctaccgcacgtacgcagcatgcccgtcagggccataacttcttcaatttgatcgccagcgcaaatcagggtagtgcgaccgctcgcgtcgatggtacgcagatcggtacgcgccaacaaggaggtttcgctacacgccggcagttccataccgtgggtgacctcaatcgtctcacccgacagtgccgcgctctcagcagaagccagaaacacagccgcatcagcaacatcgccaacggacggaaagcggcgttccaaagcgccttggtcgtttgcgcggcacaggcgcatggtgttcaaaaagtgatgggcggtgtcgccctccgggcgacccttcagttgatccatacgttgaaacacggtacggatgcggtcggactcaataggacctgggaaaatagtgttaacgcggatgccacgagcgcccagctcgcgggcagccagctgggacaaagcgttcagcgctgccttcggggtaacgtacggaatgcgaccatagtactctgcgcgcgagaaaatagtcgaaacgttgataactgcggagccaactggcatgtgcggagccgcaatacgcatcaggtgccaacccatgcccaacagattcgcgatcgaagcgtgcaaagtctcctccgcacccggacccaattctgcctccgtcagcggaatttccgccaagcgacgttgggcaccggcggagcccgcgttgttaaccagaatgtcaatctgaccgtggcgagcgacgattgcctcgataccggcacggacagccacagggtcggaaccatccatcacctccaaatcaatgcgcttggccggcacgccggcctcggcttgcatgcgctcggccagcgcggtcagcttcgcacggttacggccgctgataataaccgtcgcaccctcggccagaaaacggcgggtcagctcggaaccaatgttaccagcaccaccggtgatcaatgcaattttaccagccaaacgacccgtaccggccatgatcgtttcgcctgtggtatgaaatttcacacgcattatatacaaaaaaagcgattcagaccccgttggcaagccgcgtggttaactcatggtaattctccacgcttataagcgaataaaggaagatggccgccccgcagggcagcaggtctgtgaaacagtatagagattcatcggcacaaaggctttgctttttgtcatttattcaaaccttcaagcgattcagatagcgccagcttaatcggttcaacagcgaaggtcagccccttttcgccgttgtccgcgacaacataacgcagtgcaccttctgtctcggtgtaataacgtttgtttttccccgccgttcccaagggcgaattccacattggtcgctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcattagtgtccttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 130 - DNA, 2404 bp, artificial sequence
  chemically synthesized
aacgaattcaagcttgatatcattcaggacgagcctcagactccagcgtaactggactgaaaacaaactaaagcgcccttgtggcgctttagttttgttccgcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccccctcaagtcaaaagcctccggtcggaggcttttgactttctgctatggaggtcaggtatgatttaaatggtcagtattgagcgatatctagagaattcgtc

SEQ ID NO: 131 - DNA, 21 bp, artificial sequence
  chemically synthesized
aacgaattcaagcttgatatc

SEQ ID NO: 132 - DNA, 21 bp, artificial sequence
  chemically synthesized
gaattcgttgacgaattctct

SEQ ID NO: 133 - DNA, 24 bp, artificial sequence
  chemically synthesized
ggaaacagctatgaccatgattac

SEQ ID NO: 134 - DNA, 26 bp, artificial sequence
  chemically synthesized
ttgtaaaacgacggccagtgagcgcg

SEQ ID NO: 135 - DNA, 6678 bp, artificial sequence
  chemically synthesized
ttaaaacgacggccagtgagcgcgcgtaatacgactcactatagggcgaattggagctcccgcggtgcggccgctctagaactagtggatcccccgggctgcagcgaccaatgtggaattcgcccttgggaacggcggggaaaaacaaacgttattacaccgagacagaaggtgcactgcgttatgttgtcgcggacaacggcgaaaaggggctgaccttcgctgttgaaccgattaagctggcgctatctgaatcgcttgaaggtttgaataaatgacaaaaagcaaagcctttgtgccgatgaatctctatactgtttcacagacctgctgccctgcggggcggccatcttcctttattcgcttataagcgtggagaattaccatgagttaaccacgcggcttgccaacggggtctgaatcgctttttttgtatataatgcgtgtgaaatttcataccacaggcgaaacgatcatggccggtacgggtcgtttggctggtaaaattgcattgatcaccggtggtgctggtaacattggttccgagctgacccgccgttttctggccgagggtgcgacggttattatcagcggccgtaaccgtgcgaagctgaccgcgctggccgagcgcatgcaagccgaggccggcgtgccggccaagcgcattgatttggaggtgatggatggttccgaccctgtggctgtccgtgccggtatcgaggcaatcgtcgctcgccacggtcagattgacattctggttaacaacgcgggctccgccggtgcccaacgtcgcttggcggaaattccgctgacggaggcagaattgggtccgggtgcggaggagactttgcacgcttcgatcgcgaatctgttgggcatgggttggcacctgatgcgtattgcggctccgcacatgccagttggctccgcagttatcaacgtttcgactattttctcgcgcgcagagtactatggtcgcattccgtacgttaccccgaaggcagcgctgaacgctttgtcccagctggctgcccgcgagctgggcgctcgtggcatccgcgttaacactattttcccaggtcctattgagtccgaccgcatccgtaccgtgtttcaacgtatggatcaactgaagggtcgcccggagggcgacaccgcccatcactttttgaacaccatgcgcctgtgccgcgcaaacgaccaaggcgctttggaacgccgctttccgtccgttggcgatgttgctgatgcggctgtgtttctggcttctgctgagagcgcggcactgtcgggtgagacgattgaggtcacccacggtatggaactgccggcgtgtagcgaaacctccttgttggcgcgtaccgatctgcgtaccatcgacgcgagcggtcgcactaccctgatttgcgctggcgatcaaattgaagaagttatggccctgacgggcatgctgcgtacgtgcggtagcgaagtgattatcggcttccgttctgcggctgccctggcgcaatttgagcaggcagtgaatgaatctcgccgtctggcaggtgcggatttcaccccgccgatcgctttgccgttggacccacgtgacccggccaccattgatgcggttttcgattggggcgcaggcgagaatacgggtggcatccatgcggcggtcattctgccggcaacctcccacgaaccggctccgtgcgtgattgaagtcgatgacgaacgcgtcctgaatttcctggccgatgaaattaccggcaccatcgttattgcgagccgtttggcgcgctattggcaatcccaacgcctgaccccgggtgcccgtgcccgcggtccgcgtgttatctttctgagcaacggtgccgatcaaaatggtaatgtttacggtcgtattcaatctgcggcgatcggtcaattgattcgcgtttggcgtcacgaggcggagttggactatcaacgtgcatccgccgcaggcgatcacgttctgccgccggtttgggcgaaccagattgtccgtttcgctaaccgctccctggaaggtctggagttcgcgtgcgcgtggaccgcacagctgctgcacagccaacgtcatattaacgaaattacgctgaacattccagccaatattagcgcgaccacgggcgcacgttccgccagcgtcggctgggccgagtccttgattggtctgcacctgggcaaggtggctctgattaccggtggttcggcgggcatcggtggtcaaatcggtcgtctgctggccttgtctggcgcgcgtgtgatgctggccgctcgcgatcgccataaattggaacagatgcaagccatgattcaaagcgaattggcggaggttggttataccgatgtggaggaccgtgtgcacatcgctccgggttgcgatgtgagcagcgaggcgcagctggcagatctggtggaacgtacgctgtccgcattcggtaccgtggattatttgattaataacgccggtattgcgggcgtggaggagatggtgatcgacatgccggtggaaggctggcgtcacaccctgtttgccaacctgatttcgaattattcgctgatgcgcaagttggcgccgctgatgaagaagcaaggtagcggttacatcctgaacgtttcttcctattttggcggtgagaaggacgcggcgattccttatccgaaccgcgccgactacgccgtctccaaggctggccaacgcgcgatggcggaagtgttcgctcgtttcctgggtccagagattcagatcaatgctattgccccaggtccggttgaaggcgaccgcctgcgtggtaccggtgagcgtccgggcctgtttgctcgtcgcgcccgtctgatcttggagaataaacgcctgaacgaattgcacgcggctttgattgctgcggcccgcaccgatgagcgctcgatgcacgagttggttgaattgttgctgccgaacgacgtggccgcgttggagcagaacccagcggcccctaccgcgctgcgtgagctggcacgccgcttccgtagcgaaggtgatccggcggcaagctcctcgtccgccttgctgaatcgctccatcgctgccaagctgttggctcgcttgcataacggtggctatgtgctgccggcggatatttttgcaaatctgcctaatccgccggacccgttctttacccgtgcgcaaattgaccgcgaagctcgcaaggtgcgtgatggtattatgggtatgctgtatctgcagcgtatgccaaccgagtttgacgtcgctatggcaaccgtgtactatctggccgatcgtaacgtgagcggcgaaactttccatccgtctggtggtttgcgctacgagcgtaccccgaccggtggcgagctgttcggcctgccatcgccggaacgtctggcggagctggttggtagcacggtgtacctgatcggtgaacacctgaccgagcacctgaacctgctggctcgtgcctatttggagcgctacggtgcccgtcaagtggtgatgattgttgagacggaaaccggtgcggaaaccatgcgtcgtctgttgcatgatcacgtcgaggcaggtcgcctgatgactattgtggcaggtgatcagattgaggcagcgattgaccaagcgatcacgcgctatggccgtccgggtccggtggtgtgcactccattccgtccactgccaaccgttccgctggtcggtcgtaaagactccgattggagcaccgttttgagcgaggcggaatttgcggaactgtgtgagcatcagctgacccaccatttccgtgttgctcgtaagatcgccttgtcggatggcgcgtcgctggcgttggttaccccggaaacgactgcgactagcaccacggagcaatttgctctggcgaacttcatcaagaccaccctgcacgcgttcaccgcgaccatcggtgttgagtcggagcgcaccgcgcaacgtattctgattaaccaggttgatctgacgcgccgcgcccgtgcggaagagccgcgtgacccgcacgagcgtcagcaggaattggaacgcttcattgaagccgttctgctggttaccgctccgctgcctcctgaggcagacacgcgctacgcaggccgtattcaccgcggtcgtgcgattaccgtctaatccaagggcgaattccacagtggatatcaagcttatcgataccgtcgacctcgagggggggcccggtacccagcttttgttccctttagtgagggttaattgcgcgcttggcgtaatcatggtcatagctgtttccaacgaattcaagcttgatatcattcaggacgagcctcagactccagcgtaactggactgaaaacaaactaaagcgcccttgtggcgctttagttttgttccgcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagtacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccccctcaagtcaaaagcctccggtcggaggcttttgactttctgctatggaggtcaggtatgatttaaatggtcagtattgagcgatatctagagaattcgtc

SEQ ID NO: 136 - DNA, 21 bp, artificial sequence
  chemically synthesized
gagcacagtatcgcaaacatg

SEQ ID NO: 137 - DNA, 25 bp, artificial sequence
  chemically synthesized
caggcagcgcatcaggcagccctgg

SEQ ID NO: 138 - DNA, 23 bp, artificial sequence
  chemically synthesized
agcaggcaccagcggtaagcttg

SEQ ID NO: 139 - DNA, 25 bp, artificial sequence
  chemically synthesized
aacagtccttgttacgtctgtgtgg

SEQ ID NO: 140 - DNA, 23 bp, artificial sequence
  chemically synthesized
aaaattgcccgtttgtgaaccac

SEQ ID NO: 141 - DNA, 23 bp, artificial sequence
  chemically synthesized
atcattggcagccatttcggttc

SEQ ID NO: 142 - DNA, 23 bp, artificial sequence
  chemically synthesized
gaaattgtggcgatttatcgcgc

SEQ ID NO: 143 - DNA, 24 bp, artificial sequence
  chemically synthesized
cccagaaacgtacttctgttggcg

SEQ ID NO: 144 - DNA, 22 bp, artificial sequence
  chemically synthesized
ggcggcaagtgagcgaatcccg

SEQ ID NO: 145 - DNA, 22 bp, artificial sequence
  chemically synthesized
cgcttgcgccaaagccgatgcg

SEQ ID NO: 146 - DNA, 22 bp, artificial sequence
  chemically synthesized
tttatcgatattgatccaggtg

SEQ ID NO: 147 - DNA, 24 bp, artificial sequence
  chemically synthesized
gtgtgcattacccaacggcaaacg

SEQ ID NO: 148 - DNA, 21 bp, artificial sequence
  chemically synthesized
atcacctggggtcagttggcg

SEQ ID NO: 149 - DNA, 23 bp, artificial sequence
  chemically synthesized
cgtcgttcatctgtttgagatcg

SEQ ID NO: 150 - DNA, 23 bp, artificial sequence
  chemically synthesized
ccagcgtggctacaacattgaaa

SEQ ID NO: 151 - DNA, 22 bp, artificial sequence
  chemically synthesized
tcccactgaaaggagtttacgg

SEQ ID NO: 152 - DNA, 24 bp, artificial sequence
  chemically synthesized
gcatcgcgctattgaatcaggccg

SEQ ID NO: 153 - DNA, 24 bp, artificial sequence
  chemically synthesized
cgtcatgcaccactaactgtcttg

SEQ ID NO: 154 - DNA, 24 bp, artificial sequence
  chemically synthesized
gcgtgaagcaatggcttatgccca

SEQ ID NO: 155 - DNA, 22 bp, artificial sequence
  chemically synthesized
caaaaataagcactcccagtgc

SEQ ID NO: 156 - DNA, 22 bp, artificial sequence
  chemically synthesized
ggcggcaagtgagcgaatcccg

SEQ ID NO: 157 - DNA, 22 bp, artificial sequence
  chemically synthesized
cgcttgcgccaaagccgatgcg

SEQ ID NO: 158 - DNA, 20 bp, artificial sequence
  chemically synthesized
cagtcatagccgaatagcct

SEQ ID NO: 159 - DNA, 8252 bp, artificial sequence; identical to SEQ ID NO: 123
  chemically synthesized plasmid comprising codon optimized mcr gene

SEQ ID NO: 160 - DNA, 7988 bp, artificial sequence
  pHT08 plasmid
ctcgagggtaactagcctcgccgatcccgcaagaggcccggcagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcatgcttaagttattggtatgactggttttaagcgcaaaaaaagttgctttttcgtacctattaatgtatcgttttagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaagaccacattaaaaaatgtggtcttttgtgtttttttaaaggatttgagcgtagcgaaaaatccttttctttcttatcttgataataagggtaactattgccgatcgtccattccgacagcatcgccagtcactatggcgtgctgctagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcaggccttaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccacttttcccgcgtttgcagaaacgtggctggcctggttcaccacgcgggaaacggtctgataagagacaccggcatactctgcgacatcgtataacgttactggtttcatcaaaatcgtctccctccgtttgaatatttgattgatcgtaaccagatgaagcactctttccactatccctacagtgttatggcttgaacaatcacgaaacaataattggtacgtacgatctttcagccgactcaaacatcaaatcttacaaatgtagtctttgaaagtattacatatgtaagatttaaatgcaaccgttttttcggaaggaaatgatgacctcgtttccaccggaattagcttggtaccagctattgtaacataatcggtacgggggtgaaaaagctaacggaaaagggagcggaaaagaatgatgtaagcgtgaaaaattttttatcttatcacttgaaattggaagggagattctttattataagaattgtggaattgtgagcggataacaattcccaattaaaggaggaaggatctatgcgcggaagccatcaccatcaccatcaccatcacggatcctctagagtcgacgtccccggggcagcccgcctaatgagcgggcttttttcacgtcacgcgtccatggagatctttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaactattcataaccttacatggaattgatcgaagggtggaaggttaatggtacgaaattaggggatctacctagaaagcacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcacatgtaaagaaggctttaaaaaaggaaaaagagggtcttccaaaggttttgaaaaaagagcacgatgaatttgttaaaaaaatgatggatgagtcagaaacaattaatattccagaggccttacaatatgacacaatgtatgaagatatactcagtaaaggagaaattcgaaaagaaatcaaaaaacaaatacctaatcctacaacatcttttgagagtatatcaatgacaactgaagaggaaaaagtcgacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagat

SEQ ID NO: 161 - DNA, 27 bp, artificial sequence
  chemically synthesized
ggaaggatccatgtccggtacgggtcg

SEQ ID NO: 162 - DNA, 26 bp, artificial sequence
  chemically synthesized
gggattagacggtaatcgcacgaccg

SEQ ID NO: 163 - DNA, 7794 bp, artificial sequence
  chemically synthesized
ggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtcaccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatttttttttcctcctcggcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggcgctgacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccatttttttttcctcctttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatttttttttcctcctttaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattgaggggtgtcgcccttattcgactctatagtgaagttcctattctctagaaagtataggaacttctgaagtggggtttaaactccctctgcccttccctcccgcttcatccttatttttggacaataaactagagaacaatttgaacttgaattggaattcagattcagagcaagagacaagaaacttccctttttcttctccacatattattatttattcgtgtattttcttttaacgatacgatacgatacgacacgatacgatacgacacgctactatacagtgacgtcagattgtactgagagtgcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacaggtgtgaaataccgcacagtcatgagatccgataacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaacggatccgaatactagttggccaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggccaccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagggatgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataagtttaaacaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgct

SEQ ID NO: 164 - DNA, 7794 bp, artificial sequence; identical to SEQ ID NO: 163
  chemically synthesized

SEQ ID NO: 165 - DNA, 6477 bp, artificial sequence
  chemically synthesized
aaactccctctgcccttccctcccgcttcatccttatttttggacaataaactagagaacaatttgaacttgaattggaattcagattcagagcaagagacaagaaacttccctttttcttctccacatattattatttattcgtgtattttcttttaacgatacgatacgatacgacacgatacgatacgacacgctactatacagtgacgtcagattgtactgagagtgcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacaggtgtgaaataccgcacagtcatgagatccgataacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaacggatccatggccggtacgggtcgtttggctggtaaaattgcattgatcaccggtggtgctggtaacattggttccgagctgacccgccgttttctggccgagggtgcgacggttattatcagcggccgtaaccgtgcgaagctgaccgcgctggccgagcgcatgcaagccgaggccggcgtgccggccaagcgcattgatttggaggtgatggatggttccgaccctgtggctgtccgtgccggtatcgaggcaatcgtcgctcgccacggtcagattgacattctggttaacaacgcgggctccgccggtgcccaacgtcgcttggcggaaattccgctgacggaggcagaattgggtccgggtgcggaggagactttgcacgcttcgatcgcgaatctgttgggcatgggttggcacctgatgcgtattgcggctccgcacatgccagttggctccgcagttatcaacgtttcgactattttctcgcgcgcagagtactatggtcgcattccgtacgttaccccgaaggcagcgctgaacgctttgtcccagctggctgcccgcgagctgggcgctcgtggcatccgcgttaacactattttcccaggtcctattgagtccgaccgcatccgtaccgtgtttcaacgtatggatcaactgaagggtcgcccggagggcgacaccgcccatcactttttgaacaccatgcgcctgtgccgcgcaaacgaccaaggcgctttggaacgccgctttccgtccgttggcgatgttgctgatgcggctgtgtttctggcttctgctgagagcgcggcactgtcgggtgagacgattgaggtcacccacggtatggaactgccggcgtgtagcgaaacctccttgttggcgcgtaccgatctgcgtaccatcgacgcgagcggtcgcactaccctgatttgcgctggcgatcaaattgaagaagttatggccctgacgggcatgctgcgtacgtgcggtagcgaagtgattatcggcttccgttctgcggctgccctggcgcaatttgagcaggcagtgaatgaatctcgccgtctggcaggtgcggatttcaccccgccgatcgctttgccgttggacccacgtgacccggccaccattgatgcggttttcgattggggcgcaggcgagaatacgggtggcatccatgcggcggtcattctgccggcaacctcccacgaaccggctccgtgcgtgattgaagtcgatgacgaacgcgtcctgaatttcctggccgatgaaattaccggcaccatcgttattgcgagccgtttggcgcgctattggcaatcccaacgcctgaccccgggtgcccgtgcccgcggtccgcgtgttatctttctgagcaacggtgccgatcaaaatggtaatgtttacggtcgtattcaatctgcggcgatcggtcaattgattcgcgtttggcgtcacgaggcggagttggactatcaacgtgcatccgccgcaggcgatcacgttctgccgccggtttgggcgaaccagattgtccgtttcgctaaccgctccctggaaggtctggagttcgcgtgcgcgtggaccgcacagctgctgcacagccaacgtcatattaacgaaattacgctgaacattccagccaatattagcgcgaccacgggcgcacgttccgccagcgtcggctgggccgagtccttgattggtctgcacctgggcaaggtggctctgattaccggtggttcggcgggcatcggtggtcaaatcggtcgtctgctggccttgtctggcgcgcgtgtgatgctggccgctcgcgatcgccataaattggaacagatgcaagccatgattcaaagcgaattggcggaggttggttataccgatgtggaggaccgtgtgcacatcgctccgggttgcgatgtgagcagcgaggcgcagctggcagatctggtggaacgtacgctgtccgcattcggtaccgtggattatttgattaataacgccggtattgcgggcgtggaggagatggtgatcgacatgccggtggaaggctggcgtcacaccctgtttgccaacctgatttcgaattattcgctgatgcgcaagttggcgccgctgatgaagaagcaaggtagcggttacatcctgaacgtttcttcctattttggcggtgagaaggacgcggcgattccttatccgaaccgcgccgactacgccgtctccaaggctggccaacgcgcgatggcggaagtgttcgctcgtttcctgggtccagagattcagatcaatgctattgccccaggtccggttgaaggcgaccgcctgcgtggtaccggtgagcgtccgggcctgtttgctcgtcgcgcccgtctgatcttggagaataaacgcctgaacgaattgcacgcggctttgattgctgcggcccgcaccgatgagcgctcgatgcacgagttggttgaattgttgctgccgaacgacgtggccgcgttggagcagaacccagcggcccctaccgcgctgcgtgagctggcacgccgcttccgtagcgaaggtgatccggcggcaagctcctcgtccgccttgctgaatcgctccatcgctgccaagctgttggctcgcttgcataacggtggctatgtgctgccggcggatatttttgcaaatctgcctaatccgccggacccgttctttacccgtgcgcaaattgaccgcgaagctcgcaaggtgcgtgatggtattatgggtatgctgtatctgcagcgtatgccaaccgagtttgacgtcgctatggcaaccgtgtactatctggccgatcgtaacgtgagcggcgaaactttccatccgtctggtggtttgcgctacgagcgtaccccgaccggtggcgagctgttcggcctgccatcgccggaacgtctggcggagctggttggtagcacggtgtacctgatcggtgaacacctgaccgagcacctgaacctgctggctcgtgcctatttggagcgctacggtgcccgtcaagtggtgatgattgttgagacggaaaccggtgcggaaaccatgcgtcgtctgttgcatgatcacgtcgaggcaggtcgcctgatgactattgtggcaggtgatcagattgaggcagcgattgaccaagcgatcacgcgctatggccgtccgggtccggtggtgtgcactccattccgtccactgccaaccgttccgctggtcggtcgtaaagactccgattggagcaccgttttgagcgaggcggaatttgcggaactgtgtgagcatcagctgacccaccatttccgtgttgctcgtaagatcgccttgtcggatggcgcgtcgctggcgttggttaccccggaaacgactgcgactagcaccacggagcaatttgctctggcgaacttcatcaagaccaccctgcacgcgttcaccgcgaccatcggtgttgagtcggagcgcaccgcgcaacgtattctgattaaccaggttgatctgacgcgccgcgcccgtgcggaagagccgcgtgacccgcacgagcgtcagcaggaattggaacgcttcattgaagccgttctgctggttaccgctccgctgcctcctgaggcagacacgcgctacgcaggccgtattcaccgcggtcgtgcgattaccgtcggatctagatctcaccatcaccaccattaaactagttggccaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggccaccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagggatgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataagttt

SEQ ID NO: 166 - DNA, 6233 bp, artificial sequence
  chemically synthesized yeast plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacaatgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatgagtgtgcgtaatgagttgtaaaattatgtataaacctactttctctcacaagttatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcactaagaaaccattattatcatgacattaacctataaaaataggcgtatcacgaggccctttcgtc

SEQ ID NO: 167 - DNA, 12710 bp, artificial sequence
  chemically synthesized plasmid comprising codon optimized mcr gene
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacaatgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatgagtgtgcgtaatgagttgtaaaattatgtataaacctactttctctcacaagttatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccaaactccctctgcccttccctcccgcttcatccttatttttggacaataaactagagaacaatttgaacttgaattggaattcagattcagagcaagagacaagaaacttccctttttcttctccacatattattatttattcgtgtattttcttttaacgatacgatacgatacgacacgatacgatacgacacgctactatacagtgacgtcagattgtactgagagtgcagattgtactgagagtgcaccataaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatgcggtgtgaaataccgcacaggtgtgaaataccgcacagtcatgagatccgataacttcttttctttttttttcttttctctctcccccgttgttgtctcaccatatccgcaatgacaaaaaaaatgatggaagacactaaaggaaaaaattaacgacaaagacagcaccaacagatgtcgttgttccagagctgatgaggggtatcttcgaacacacgaaactttttccttccttcattcacgcacactactctctaatgagcaacggtatacggccttccttccagttacttgaatttgaaataaaaaaagtttgccgctttgctatcaagtataaatagacctgcaattattaatcttttgtttcctcgtcattgttctcgttccctttcttccttgtttctttttctgcacaatatttcaagctataccaagcatacaatcaactccaacggatccatggccggtacgggtcgtttggctggtaaaattgcattgatcaccggtggtgctggtaacattggttccgagctgacccgccgttttctggccgagggtgcgacggttattatcagcggccgtaaccgtgcgaagctgaccgcgctggccgagcgcatgcaagccgaggccggcgtgccggccaagcgcattgatttggaggtgatggatggttccgaccctgtggctgtccgtgccggtatcgaggcaatcgtcgctcgccacggtcagattgacattctggttaacaacgcgggctccgccggtgcccaacgtcgcttggcggaaattccgctgacggaggcagaattgggtccgggtgcggaggagactttgcacgcttcgatcgcgaatctgttgggcatgggttggcacctgatgcgtattgcggctccgcacatgccagttggctccgcagttatcaacgtttcgactattttctcgcgcgcagagtactatggtcgcattccgtacgttaccccgaaggcagcgctgaacgctttgtcccagctggctgcccgcgagctgggcgctcgtggcatccgcgttaacactattttcccaggtcctattgagtccgaccgcatccgtaccgtgtttcaacgtatggatcaactgaagggtcgcccggagggcgacaccgcccatcactttttgaacaccatgcgcctgtgccgcgcaaacgaccaaggcgctttggaacgccgctttccgtccgttggcgatgttgctgatgcggctgtgtttctggcttctgctgagagcgcggcactgtcgggtgagacgattgaggtcacccacggtatggaactgccggcgtgtagcgaaacctccttgttggcgcgtaccgatctgcgtaccatcgacgcgagcggtcgcactaccctgatttgcgctggcgatcaaattgaagaagttatggccctgacgggcatgctgcgtacgtgcggtagcgaagtgattatcggcttccgttctgcggctgccctggcgcaatttgagcaggcagtgaatgaatctcgccgtctggcaggtgcggatttcaccccgccgatcgctttgccgttggacccacgtgacccggccaccattgatgcggttttcgattggggcgcaggcgagaatacgggtggcatccatgcggcggtcattctgccggcaacctcccacgaaccggctccgtgcgtgattgaagtcgatgacgaacgcgtcctgaatttcctggccgatgaaattaccggcaccatcgttattgcgagccgtttggcgcgctattggcaatcccaacgcctgaccccgggtgcccgtgcccgcggtccgcgtgttatctttctgagcaacggtgccgatcaaaatggtaatgtttacggtcgtattcaatctgcggcgatcggtcaattgattcgcgtttggcgtcacgaggcggagttggactatcaacgtgcatccgccgcaggcgatcacgttctgccgccggtttgggcgaaccagattgtccgtttcgctaaccgctccctggaaggtctggagttcgcgtgcgcgtggaccgcacagctgctgcacagccaacgtcatattaacgaaattacgctgaacattccagccaatattagcgcgaccacgggcgcacgttccgccagcgtcggctgggccgagtccttgattggtctgcacctgggcaaggtggctctgattaccggtggttcggcgggcatcggtggtcaaatcggtcgtctgctggccttgtctggcgcgcgtgtgatgctggccgctcgcgatcgccataaattggaacagatgcaagccatgattcaaagcgaattggcggaggttggttataccgatgtggaggaccgtgtgcacatcgctccgggttgcgatgtgagcagcgaggcgcagctggcagatctggtggaacgtacgctgtccgcattcggtaccgtggattatttgattaataacgccggtattgcgggcgtggaggagatggtgatcgacatgccggtggaaggctggcgtcacaccctgtttgccaacctgatttcgaattattcgctgatgcgcaagttggcgccgctgatgaagaagcaaggtagcggttacatcctgaacgtttcttcctattttggcggtgagaaggacgcggcgattccttatccgaaccgcgccgactacgccgtctccaaggctggccaacgcgcgatggcggaagtgttcgctcgtttcctgggtccagagattcagatcaatgctattgccccaggtccggttgaaggcgaccgcctgcgtggtaccggtgagcgtccgggcctgtttgctcgtcgcgcccgtctgatcttggagaataaacgcctgaacgaattgcacgcggctttgattgctgcggcccgcaccgatgagcgctcgatgcacgagttggttgaattgttgctgccgaacgacgtggccgcgttggagcagaacccagcggcccctaccgcgctgcgtgagctggcacgccgcttccgtagcgaaggtgatccggcggcaagctcctcgtccgccttgctgaatcgctccatcgctgccaagctgttggctcgcttgcataacggtggctatgtgctgccggcggatatttttgcaaatctgcctaatccgccggacccgttctttacccgtgcgcaaattgaccgcgaagctcgcaaggtgcgtgatggtattatgggtatgctgtatctgcagcgtatgccaaccgagtttgacgtcgctatggcaaccgtgtactatctggccgatcgtaacgtgagcggcgaaactttccatccgtctggtggtttgcgctacgagcgtaccccgaccggtggcgagctgttcggcctgccatcgccggaacgtctggcggagctggttggtagcacggtgtacctgatcggtgaacacctgaccgagcacctgaacctgctggctcgtgcctatttggagcgctacggtgcccgtcaagtggtgatgattgttgagacggaaaccggtgcggaaaccatgcgtcgtctgttgcatgatcacgtcgaggcaggtcgcctgatgactattgtggcaggtgatcagattgaggcagcgattgaccaagcgatcacgcgctatggccgtccgggtccggtggtgtgcactccattccgtccactgccaaccgttccgctggtcggtcgtaaagactccgattggagcaccgttttgagcgaggcggaatttgcggaactgtgtgagcatcagctgacccaccatttccgtgttgctcgtaagatcgccttgtcggatggcgcgtcgctggcgttggttaccccggaaacgactgcgactagcaccacggagcaatttgctctggcgaacttcatcaagaccaccctgcacgcgttcaccgcgaccatcggtgttgagtcggagcgcaccgcgcaacgtattctgattaaccaggttgatctgacgcgccgcgcccgtgcggaagagccgcgtgacccgcacgagcgtcagcaggaattggaacgcttcattgaagccgttctgctggttaccgctccgctgcctcctgaggcagacacgcgctacgcaggccgtattcaccgcggtcgtgcgattaccgtcggatctagatctcaccatcaccaccattaaactagttggccaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggccaccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagggatgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataagtttgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcactaagaaaccattattatcatgacattaacctataaaaataggcgtatcacgaggccctttcgtc

SEQ ID NO: 168 - DNA, 747 bp, Escherichia coli
atgatcgttttagtaactggagcaacggcaggttttggtgaatgcattactcgtcgttttattcaacaagggcataaagttatcgccactggccgtcgccaggaacggttgcaggagttaaaagacgaactgggagataatctgtatatcgcccaactggacgttcgcaaccgcgccgctattgaagagatgctggcatcgcttcctgccgagtggtgcaatattgatatcctggtaaataatgccggcctggcgttgggcatggagcctgcgcataaagccagcgttgaagactgggaaacgatgattgataccaacaacaaaggcctggtatatatgacgcgcgccgtcttaccgggtatggttgaacgtaatcatggtcatattattaacattggctcaacggcaggtagctggccgtatgccggtggtaacgtttacggtgcgacgaaagcgtttgttcgtcagtttagcctgaatctgcgtacggatctgcatggtacggcggtgcgcgtcaccgacatcgaaccgggtctggtgggtggtaccgagttttccaatgtccgctttaaaggcgatgacggtaaagcagaaaaaacctatcaaaataccgttgcattgacgccagaagatgtcagcgaagccgtctggtgggtgtcaacgctgcctgctcacgtcaatatcaataccctggaaatgatgccggttacccaaagctatgccggactgaatgtccaccgtcagtaa

SEQ ID NO: 169 - protein, 248 aa, Escherichia coli
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

(19) United States
(12) Patent Application Publication
(10) Pub. No.: US 2013/0189787 A1
(43) Pub. Date: Jul. 25, 2013

(54) Methods, Systems And Compositions Related To Reduction Of Conversions Of Microbially Produced 3-Hydroxyproplonic Acid (3-HP) To Aldehyde Metabolites

(71) Applicants: Michael D. Lynch, Boulder, CO (US); Christopher P. Mercogliano, Boulder Superior, CO (US); Matthew L. Lipscomb, Boulder, CO (US); Tanya E.W. Lipscomb, Boulder, CO (US)

(72) Inventors: Michael D. Lynch, Boulder, CO (US); Christopher P. Mercogliano, Boulder Superior, CO (US); Matthew L. Lipscomb, Boulder, CO (US); Tanya E.W. Lipscomb, Boulder, CO (US)

(73) Assignee: OPX BIOTECHNOLOGIES ,INC.

(21) Appl. No.: 13/062,917

(22) PCT Filed: Sep. 15, 2009
(86) PCT No.: PCT/US2009/057058

§ 371 Date: May 30, 2011

Related U.S. Application Data
(60) Provisional application No. 61/096,937, filed on Sep. 15, 2008.

(51) Int. Cl. C12P 7/42; C12N 15/63

(52) U.S. Cl. CPC C12P 7/42; C12N 15/63
USPC 435/471; 435/252.3; 435/257.2; 435/254.11; 435/254.2; 435/252.34; 435/252.33; 435/252.31; 435/254.21; 435/254.23; 435/252.32; 435/254.22

(57) ABSTRACT

The present invention relates to methods, systems and compositions, including genetically modified microorganisms, directed to achieve decreased microbial conversion of 3-hydroxypropionic acid (3-HP) to aldehydes of 3-HP. In various embodiments this is achieved by disruption of particular aldehyde dehydrogenase genes, including multiple gene deletions. Among the specific nucleic acids that are deleted whereby the desired decreased conversion is achieved are aldA, aldB, puuC), and usg of E. coli. Genetically modified microorganisms so modified are adapted to produce 3-HP, such as by approaches described herein.

### RELATED APPLICATIONS

This application claims priority to the following U.S. Provisional patent application: 61/096,937, filed on Sep. 15, 2008; which is hereby incorporated by reference in its entirety.

### STATEMENT REGARDING FEDERALLY SPONSORED DEVELOPMENT

N/A

### REFERENCE TO A SEQUENCE LISTING

This application includes a sequence listing submitted electronically herewith as an ASCII text file named “3426-723-602_{—}15SEP2009_ST25.txt”, which is 281 kB in size and was created Sep. 15, 2009; the electronic sequence listing is incorporated herein by reference in its entirety. The sequences are presented in numerical order based on their respective first references in the Examples, followed by sequence numbers of sequences not recited in the Examples.

### FIELD OF THE INVENTION

The present invention relates to methods, systems and compositions, including genetically modified microorganisms, e.g., recombinant microorganisms, comprising one or more genetic modifications directed to reduce enzymatic conversion of the chemical 3-hydroxypropionic acid (3-HP) to aldehydes. Also, additional genetic modifications may be made to provide or improve one or more 3-HP biosynthesis pathways.

### BACKGROUND OF THE INVENTION

With increasing acceptance that petroleum hydrocarbon supplies are decreasing and their costs are ultimately increasing, interest has increased for developing and improving industrial microbial systems for production of chemicals and fuels. Such industrial microbial systems could completely or partially replace the use of petroleum hydrocarbons for production of certain chemicals.

One candidate chemical for biosynthesis in industrial microbial systems is 3-hydroxypropionic acid (“3-HP”, CAS No. 503-66-2), which may be converted to a number of basic building blocks, such as acrylic acid, for polymers used in a wide range of industrial and consumer products. Currently there is interest in microbial production of 3-HP.

Metabolically engineering a selected microbe is one way to work toward an economically viable industrial microbial system, such as for production of 3-HP. A great challenge in such directed metabolic engineering is determining which genetic modification(s) to incorporate, increase copy numbers of, and/or otherwise effectuate, and/or which metabolic pathways (or portions thereof) to incorporate, increase copy numbers of, decrease activity of, and/or otherwise modify in a particular target microorganism.

Metabolic engineering uses knowledge and techniques from the fields of genomics, proteomics, bioinformatics and metabolic engineering. Concomitant with designing a commercial microbial strain using metabolic engineering is the challenge to balance the overall carbon and energy flows that pass through a respective microorganism's complex and interrelated metabolic pathways and complexes.

Notwithstanding advances in these fields and in metabolic engineering as a whole, the identification of genes, enzymes, pathway portions and/or whole metabolic pathways that are related to a particular phenotype of interest remains cumbersome and at times inaccurate. Perspective as to the problem of finding a particular gene or pathway whose modification may provide greater tolerance and production of a product of interest may be further gained with the knowledge that there are at least 4,580 genes (of which 4,389 are identified as protein genes, 191 as RNA genes, and 116 as pseudo genes) and 224 identified metabolic pathways in an E. coli bacterium's genome (source www.biocyc.org, version 12.0 referring to Strain K-12). A review of specific metabolic engineering efforts, which also identifies existing gene identification and modification techniques, is “Engineering primary metabolic pathways of industrial micro-organisms,” Alexander Kern et al., Jl. of Biotechnology 129(2007)6-29, which is incorporated by reference for its listing and descriptions of such techniques.

Among the patent references that utilize metabolic engineering for 3-HP microbial production are U.S. Pat. No. 6,852,517, U.S. Pat. No. 7,186,541, U.S. Pat. No. 7,393,676, PCT Publication No. WO/2002/042418, and US/20080199926. These references utilize various approaches to genetically modify a microorganism to produce 3-HP.

Despite such interest and approaches, none of these references explicitly recognize a metabolic challenge, namely, to reduce or eliminate undesired conversions of 3-HP in the culture media and microorganism. Thus, there remains a need in the art for methods, systems and compositions to achieve such purpose.

### SUMMARY OF THE INVENTION

Some embodiments, the invention contemplates a method of making a genetically modified microorganism comprising introducing at least one genetic modification into a microorganism to decrease its enzymatic conversion of 3-hydroxypropionic acid (“3-HP”) to an aldehyde of 3-HP, wherein the genetically modified microorganism synthesizes 3-HP.

In some embodiments, the invention contemplates a method of making a genetically modified microorganism comprising: a) providing to a selected microorganism at least one genetic modification of a 3-hydroxypropionic acid (“3-HP”) production pathway to increase microbial synthesis of 3-HP above the rate of a control microorganism lacking the at least one genetic modification; and b) providing to the selected microorganism at least one genetic modification of two or more aldehyde dehydrogenases.

In some embodiments, the invention contemplates a method comprising: a) introducing to a selected microorganism at least one genetic modification of a nucleic acid sequence encoding an enzyme that is within a 50, 60, 70, 80, 90, or 95 percent homology of one of the aldehyde dehydrogenase amino acid sequences of Table 1; and b) evaluating the microorganism of step a for a difference in conversion of 3-hydroxypropionic acid (“3-HP”) to an aldehyde of 3-HP compared to a control microorganism lacking the at least one genetic modification.

In some embodiments, the invention contemplates a method of making a microorganism comprising one or more genetic modifications directed to reducing conversion of 3-hydroxypropionic acid (“3-HP”) to aldehydes comprising: a) introducing into a selected microorganism at least one genetic modification of an aldehyde dehydrogenase; b) evaluating the microorganism of step a for decreased conversion of 3-HP to an aldehyde of 3-HP; and c) optionally repeating steps a and b iteratively to obtain a microorganism comprising multiple genetic modifications directed to reducing conversion of 3-HP to aldehydes.

In some embodiments, the invention contemplates a genetically modified microorganism made by a method of the instant invention.

In some embodiments, the invention contemplates a genetically modified microorganism comprising: a) at least one genetic modification to produce 3-hydroxypropionic acid (“3-HP”); and b) at least one genetic modification of at least two aldehyde dehydrogenases effective to decrease each said aldehyde dehydrogenase's respective enzymatic activity and effective to decrease metabolism of 3-HP to any aldehydes of 3-HP, as compared to the metabolism of a control microorganism lacking the at least two genetic modifications of the aldehyde dehydrogenases.

In some embodiments, the invention contemplates a genetically modified microorganism comprising at least one genetic modification of each of two or more aldehyde dehydrogenases, said aldehyde dehydrogenases capable of converting 3-hydroxypropionic acid (“3-HP”) to any of its aldehyde metabolites.

In some embodiments, the invention contemplates a genetically modified microorganism comprising at least one genetic modification of each of at least two aldehyde dehydrogenases effective to decrease microbial enzymatic conversion of 3-hydroxypropionic acid (“3-HP”) to an aldehyde of 3-HP as compared to the enzymatic conversion of a control microorganism lacking the genetic modifications.

In some embodiments, the invention contemplates a culture system comprising: a) a population of a genetically modified microorganism as described herein; and b) a media comprising nutrients for the population.

### BRIEF DESCRIPTION OF THE DRAWINGS

FIG. 1 depicts metabolic conversions from 3-HP to a number of it aldehydes.

FIG. 2 provides, from a prior art reference, a summary of a known 3-HP production pathway from glucose to pyruvate to acetyl-CoA to malonyl-CoA to 3-HP.

FIG. 3 provides, from a prior art reference, a summary of a known 3-HP production pathway from glucose to phosphoenolpyruvate (PEP) to oxaloacetate (directly or via pyruvate) to aspartate to β-alanine to malonate semialdehyde to 3-HP.

FIG. 4A provides a summary of various 3-HP metabolic production pathways from a prior art reference.

FIG. 4B depicts propanoate metabolism map from the KEGG pathway database.

FIG. 5A provides a schematic diagram of natural mixed fermentation pathways in E. coli.

FIG. 5B provides a schematic diagram of a proposed bio-production pathway modified from FIG. 4A for production of 3-HP.

FIGS. 6-8 provide graphic data of test microorganisms' responses to 3-HP relative to control.

FIG. 9 depicts enzyme activity assays for enzymes with 3HP as substrate.

FIG. 10 provides a calibration curve for 3-HP conducted with HPLC.

FIG. 11 provides a calibration curve for 3-HP conducted for GC/MS.

Tables are provided as indicated herein and are part of the specification and including the respective examples referring to them. The identifiers “FIG.” and “Figure” are meant to refer to the respective figures.

### DETAILED DESCRIPTION OF EMBODIMENTS OF THE INVENTION

A. Introduction

The definitions and methods provided define the present invention and guide those of ordinary skill in the art in the practice of the present invention. Unless otherwise noted, terms are to be understood according to conventional usage by those of ordinary skill in the relevant art.

The present invention relates to methods, systems and compositions that are intended to improve biosynthetic capabilities of metabolically engineered microorganisms so that the latter may attain a relatively higher net productivity and/or yield in microorganisms that produce the compound 3-hydroxypropionic acid (“3-HP”, CAS No. 503-66-2). The genetic modifications, such as disruptions including deletions, are of genes that encode aldehyde dehydrogenases that convert 3-HP to an aldehyde metabolite of 3-HP. As is generally recognized by those skilled in the art, aldehyde dehydrogenases belong to a group of enzymes classified in Enzyme Classification E.C. 1.2. By making one or more such genetic modifications in a microorganism that also comprises at least one genetic modification to increase its production of 3-HP, the resulting genetically modified microorganism converts less 3-HP to one or more aldehydes of 3-HP.

Also, aspects of the invention relate to a genetically modified microorganism comprising genetic modifications to greater than one, greater than two, greater than three, or greater than four aldehyde dehydrogenases each capable of converting 3-HP to at least one of its aldehydes. Such genetic modifications typically are gene disruptions, such as gene deletions, so that less 3-HP is converted to its aldehydes.

The following sections describe aspects and features that are found in various combinations in the various embodiments of the present invention.

B. Reduction or Elimination of Undesired Aldehyde Dehydrogenase Activity in a Selected Microorganism

As to genetic modifications that reduce or eliminate undesired conversion of 3-HP to aldehydes, it is recognized that one aspect of 3-HP toxicity is a result of a particular aldehyde metabolite of 3-HP, 3-hydroxypropionaldehyde (3-HPA). 3-HPA is part of a previously characterized HPA system—a dynamic equilibrium of 3-hydroxpropionaldehyde, its hydrate and it dimer that exist together in aqueous physiologic conditions, pHs and temperatures. 3-HPA has also been termed reuterin, a known antibacterial agent produced by the gut flora Lactobacillus reuterii. 3-HPA (reuterin) is toxic to a wide range of gram negative and gram positive bacteria at concentrations as low as 15 mM (Valentine et al. Inhibitory activity spectrum of reuterin produced by Lactobacillus reuteri against intestinal bacteria, BMC Microbiol. 2007; 7: 101; Vollenweider, S. et al., Purification and Structural Characterization of 3-hydroxypropionaldehyde and its derivatives, J Agric. Food Chem., 2003, 51, 3287-3293). Genetically modified strains of E. coli capable of production of 3-HP have been characterized to also produce 3-HPA, which is known to be toxic to E. coli.

It was conceived that removal of this metabolite from 3-HP producing microorganism strains, such as via genetic modification, not only will allow for a more pure 3-HP product, but also will result in a more productive microorganism with less burden to 3-HP toxicity attributable to 3-HP's conversion to 3-HPA.

Also, in addition to the toxic effects of 3-HP that is converted to 3-HPA, the removal of the conversion capacity that converts 3-HP to various aldehydes will enable a greater flux of carbon to the desired product 3-HP which is expected to result in increased productivities and greater yields. In order to genetically manipulate organisms to greatly reduce or eliminate the conversion of 3-HP to 3-HPA and other aldehydes, it is essential to first identify the genes and enzymes responsible for such conversions. Then, genetic modification(s) to reduce or eliminate such undesired enzymatic conversion activity may result in a desired genetically modified microorganism that may be used in bio-production methods and systems that provide even greater productivity and yields of 3-HP. Such microorganism may be developed and refined by the methods, including genetic manipulations, described and/or exemplified herein.

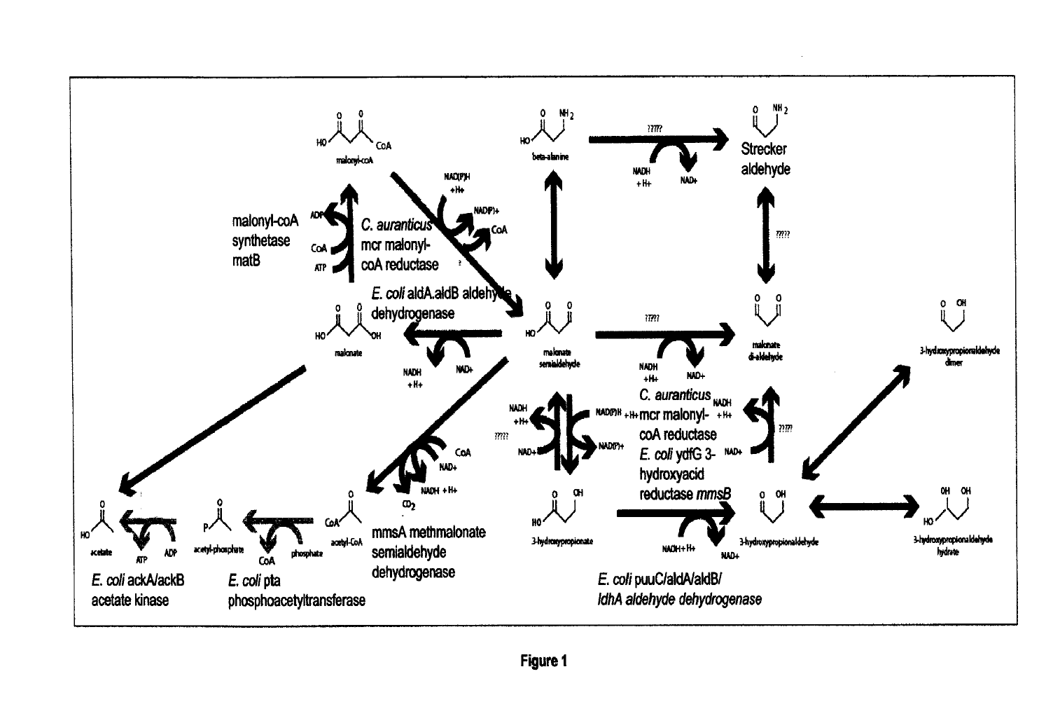

It is appreciated that various aldehyde dehydrogenases convert 3-HP to aldehyde compounds in addition to the noted 3-HPA, its dimer, and its hydrate. These include, but are not necessarily limited to, malonate semialdehyde, malonate di-aldehyde, and Strecker aldehyde (see FIG. 1). As used herein, the terms “aldehyde(s),” “aldehyde(s) of 3-HP,” “aldehyde metabolites,” and the like mean aldehyde compounds that are related by metabolic conversion from 3-HP to such aldehyde(s), such as depicted in FIG. 1.

Example 1 provides one approach to identifying genes and their enzyme products which, when their activity is reduced, such as by gene deletion, result in less conversion from 3-HP to an aldehyde. Table 1 provides a listing of these genes in E. coli, K-12 substrain MG1655, and includes the names of the proteins (enzymes) encoded and normally expressed by these genes, as provided from www.ecocyc.org, and sequence identification numbers (SEQ ID NOs.) both for the nucleic acid sequences and the encoded enzymes. This listing is meant to be exemplary and not limiting, as it is well-known that homologous genes may be identified that encode, for E. coli or other microorganism species, enzymes having similar conversion capability, i.e., converting 3-HP to an aldehyde. These may then be evaluated to determine, for a selected species, which of the homologous genes exhibit enzymatic activity to convert 3-HP to one of its aldehydes. Results of such identifications and evaluations then may be applied to modify that microorganism so as to reduce or eliminate activity of one or more such identified genes, such as by disruption, including gene deletion, and as taught herein, such modified microorganism may also comprise genetic modifications directed to 3-HP production.

Further to the determination of homologous genes in a selected microorganism species, this may be determined as follows. Using as a starting point the genes shown in Table 1, one may conduct a homology search and analysis for any of these to obtain a listing of potentially homologous sequences for the selected microorganism species. For this homology approach a local blast (http://www.ncbi.nlm.nih.gov/Tools/) (blastp) comparison using the selected set of E. coli proteins (from Table 1) is performed using different thresholds and comparing to one or more selected microorganism species (http://www.ncbi.nlm.nih.gov/genomes/lproks.cgi). A suitable E-value is chosen at least in part based on the number of results and the desired ‘tightness’ of the homology, considering the number of later evaluations to identify useful genes.

For example, search results for genes were obtained by comparing the proteins, using BLASTP, encoded by the genes of Table 1, of aldehyde dehydrogenases, with protein sequences in B. subtilis, C. necator, and Saccharomyces cerevisiae. It is noted, however, that this comparison does not include homologies for gldA, ybdH, and yghD, since no homologies were found in these three species. The criterion for inclusion in the search results is that at least one protein sequence of these species has a homology with a protein of Table 1, based on having E⁻¹⁰ or less E-value). Table 2 provides some examples of the homology relationships for genetic elements of these species that have a demonstrated homology to E. coli genes that encode enzymes of Table 1, which may be capable of catalyzing enzymatic conversion steps from 3-HP to aldehydes. Table 2 provides only a few of the many homologies obtained by these comparisons, as it was condensed by deleting the middle section (over 400 total homologies were obtained satisfying the stated criterion among the three species). Not all of the homologous sequences in such results are expected to encode a desired enzyme suitable for an enzymatic conversion step regarding 3-HP to aldehyde conversion for a target selected species that, if disrupted, would lead to less 3-HP to aldehyde conversion. However, through evaluation one or more of a combination of genetic elements known and/or expected to encode such enzymatic conversions, selected from such a listing as provided in Table 1, the most relevant genetic elements are selected for disruption. Genes so evaluated and identified for deletion in accordance with the teachings of the present invention may encode an enzyme having aldehyde dehydrogenase activity (and so be referred to as an aldehyde dehydrogenase herein), wherein that enzyme's amino acid sequence is within a 50, a 60, a 70, an 80, a 90, or a 95 percent homology of an aldehyde dehydrogenase amino acid sequence of Table 1. It is noted that such identified and evaluated nucleic acid and amino acid sequences may also be characterized by their sequence identities with the respective aldehyde dehydrogenase sequence recited herein or obtained a homology determination such as described above.

Thus, using such approaches based on identifying sequences that have a specified homology to sequences of Table 1, or other nucleic acid and amino acid sequences recited herein (“reference sequences”), nucleic acid and amino acid sequences are identified, and may be evaluated and used in embodiments of the invention, wherein the latter nucleic acid and amino acid sequences fall within a specified percentage of sequence identity.

As noted above, some embodiments of the invention comprising genetic modifications to reduce or eliminate undesired conversion of 3-HP to aldehydes also include genetic modifications that to provide and/or increase 3-HP production in a selected microorganism.

Examples 2 and 3 provide results of additional evaluations of the effects of aldehyde dehydrogenases on the conversion of 3-HP to aldhehydes of 3-HP. Example 8 describes an embodiment in which genetic modifications are made in a microorganism both to produce 3-HP and delete aldehyde dehydrogenase genes.

C. 3-HP Production

The aspects of the present invention directed to reduced or eliminated aldehyde dehydrogenase activity so as to reduce or eliminate enzymatic conversion of 3-HP to its aldehydes can be provided in a microorganism that produces 3-HP. As noted elsewhere herein, this is expected to result in an increase in productivity and/or yield of 3-HP.

As to the 3-HP production increase aspects of the invention, which may result in elevated titer of 3-HP in industrial bio-production, the genetic modifications comprise introduction of one or more nucleic acid sequences into a microorganism, wherein the one or more nucleic acid sequences encode for and express one or more production pathway enzymes (or enzymatic activities of enzymes of a production pathway). In various embodiments these improvements thereby combine to increase the efficiency and efficacy of, and consequently to lower the costs for, the industrial bio-production production of 3-HP.

Any one or more of a number of 3-HP production pathways may be used in a microorganism such as in combination with genetic modifications directed to reduce conversion of 3-HP to its aldehyde(s). In various embodiments genetic modifications are made to provide enzymatic activity for implementation of one or more of such 3-HP production pathways.

A number of 3-HP production pathways are known in the art. For example, U.S. Pat. No. 6,852,517 teaches a 3-HP production pathway from glycerol as carbon source, and is incorporated by reference for its teachings of that pathway. This reference teaches providing a genetic construct which expresses the dhaB gene from Klebsiella pneumoniae and a gene for an aldehyde dehydrogenase. These are stated to be capable of catalyzing the production of 3-HP from glycerol.

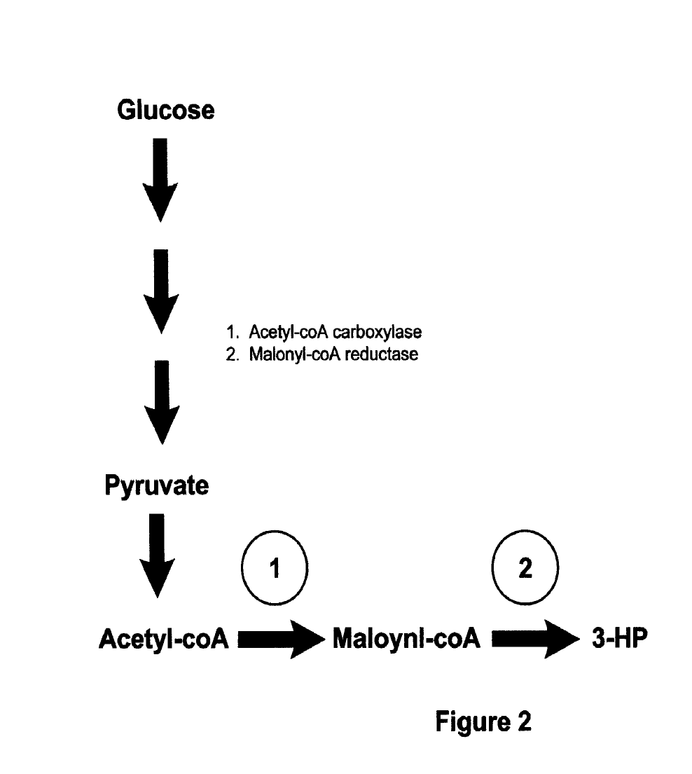

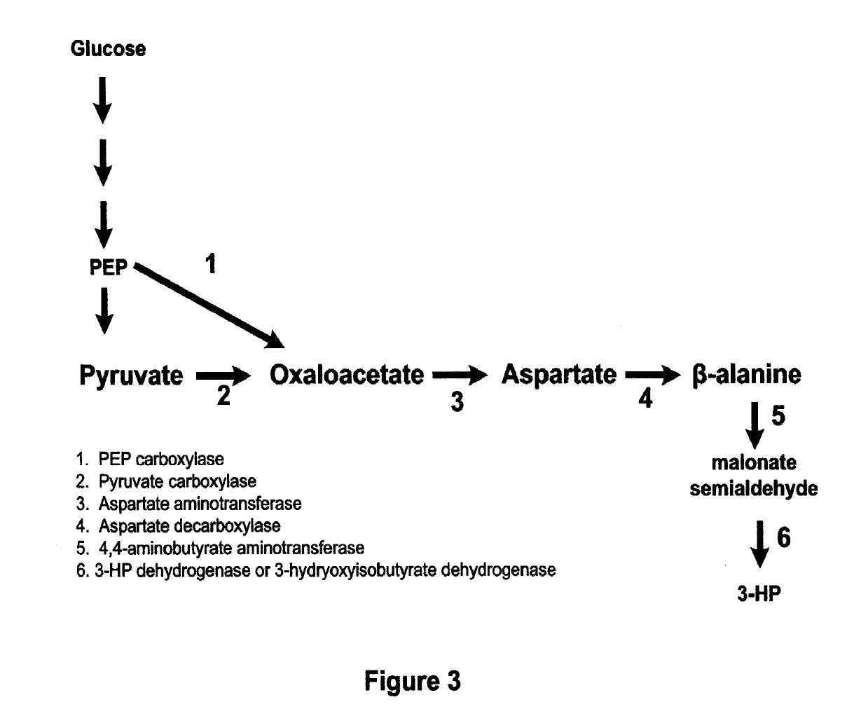

Also, WO2002/042418 (PCT/US01/43607) teaches several 3-HP production pathways. This PCT publication is incorporated by reference for its teachings of such pathways. FIG. 44 of that publication, which summarizes a 3-HP production pathway from glucose to pyruvate to acetyl-CoA to malonyl-CoA to 3-HP, is provided herein as FIG. 2. FIG. 55 of that publication, which summarizes a 3-HP production pathway from glucose to phosphoenolpyruvate (PEP) to oxaloacetate (directly or via pyruvate) to aspartate to β-alanine to malonate semialdehyde to 3-HP, is provided herein as FIG. 3. Representative enzymes for various conversions are also shown in these figures.

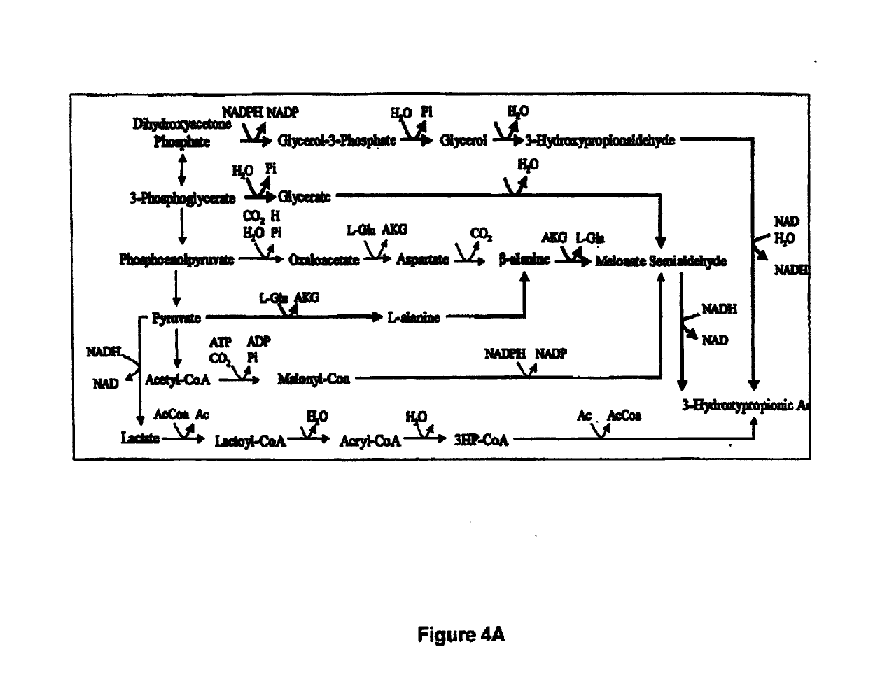

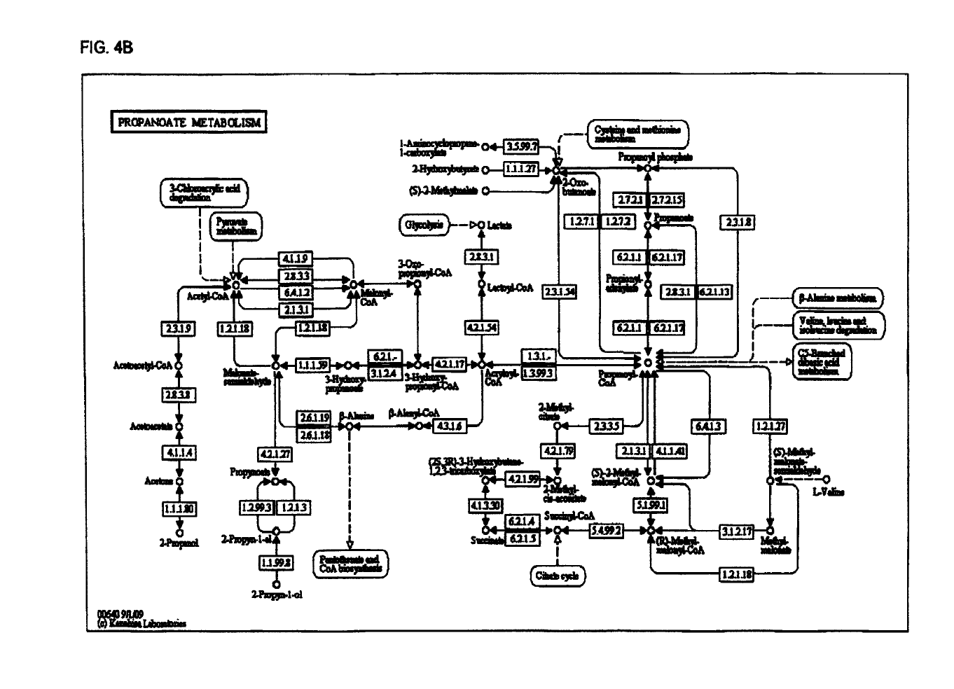

FIG. 4A, from U.S. Patent Publication No. US2008/0199926, published Aug. 21, 2008 and incorporated by reference herein, summarizes the above-described 3-HP production pathways and other known natural pathways. FIG. 4A presents several 3-HP production pathways, leading to 3-HP, many of which are also described above. FIG. 4B is the propanoate metabolism map in the KEGG pathway database (http://www.genome.jp/dbget-bin/show_pathway?map00640), and is also referenced in U.S. Patent Publication No. US2008/0199926. FIG. 4B provides a broader perspective of possible 3-HP pathways that may be completed in a selected microorganism that lacks one or more enzymes that nonetheless are known to exist in other organisms. For a selected microorganism species that lacks one or more enzymes along a metabolic pathway that leads to 3-HP (indicated as 3-Hydroxypropanoate in FIG. 4B), genetic modifications may made to provide nucleic acid sequences that encode enzymes that supply such missing activities. Thereby a 3-HP production pathway is completed in such selected microorganism. Such selected microorganism, prior to such genetic modification(s), may have been a microorganism that did not produce 3-HP, or may have been a microorganism able to produce 3-HP but at a lower production rate than following the genetic modifications. More generally as to developing specific metabolic pathways, of which many may be not found in nature, Hatzimanikatis et al. discuss this in “Exploring the diversity of complex metabolic networks,” Bioinformatics 21(8):1603-1609 (2005). This article is incorporated by reference for its teachings of the complexity of metabolic networks.

Further to the 3-HP production pathway summarized in FIG. 2, Strauss and Fuchs (“Enzymes of a novel autotrophic CO₂ fixation pathway in the phototrophic bacterium Chloroflexus aurantiacus, the 3-hydroxypropionate cycle,” Eur. J. Bichem. 215, 633-643 (1993)) identified a natural bacterial pathway that produced 3-HP. At that time the authors stated the conversion of malonyl-CoA to malonate semialdehyde was by an NADP-dependant acylating malonate semialdehyde dehydrogenase and conversion of malonate semialdehyde to 3-HP was catalyzed by a 3-hydroxypropionate dehydrogenase. However, since that time it has become appreciated that, at least for Chloroflexus aurantiacus, a single enzyme may catalyze both steps (M. Hugler et al., “Malonyl-Coenzyme A Reductase from Chloroflexus aurantiacus, a Key Enzyme of the 3-Hydroxypropionate Cycle for Autotrophic CO₂ Fixation,” J. Bacter,184(9):2404-2410 (2002)).

Accordingly, one production pathway of various embodiments of the present invention comprises malonyl-Co-A reductase enzymatic activity that achieves conversions of malonyl-CoA to malonate semialdehyde to 3-HP. As provided in the Examples section below, introduction into a microorganism of a nucleic acid sequence encoding a polypeptide providing this enzyme (or enzymatic activity) is effective to provide increased 3-HP biosynthesis.

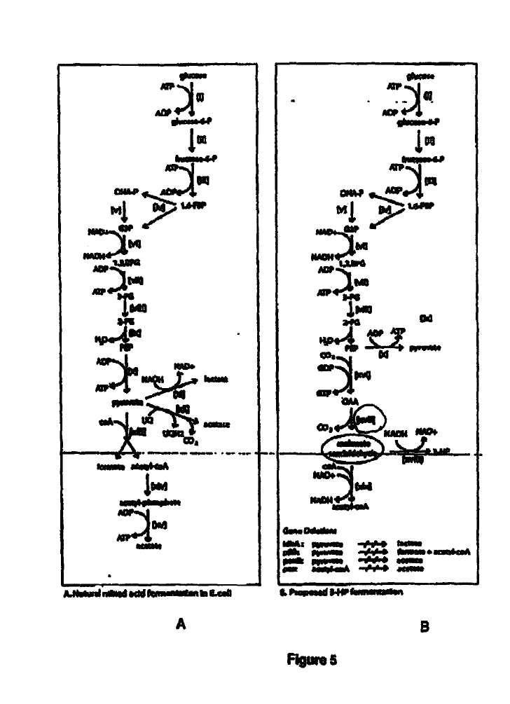

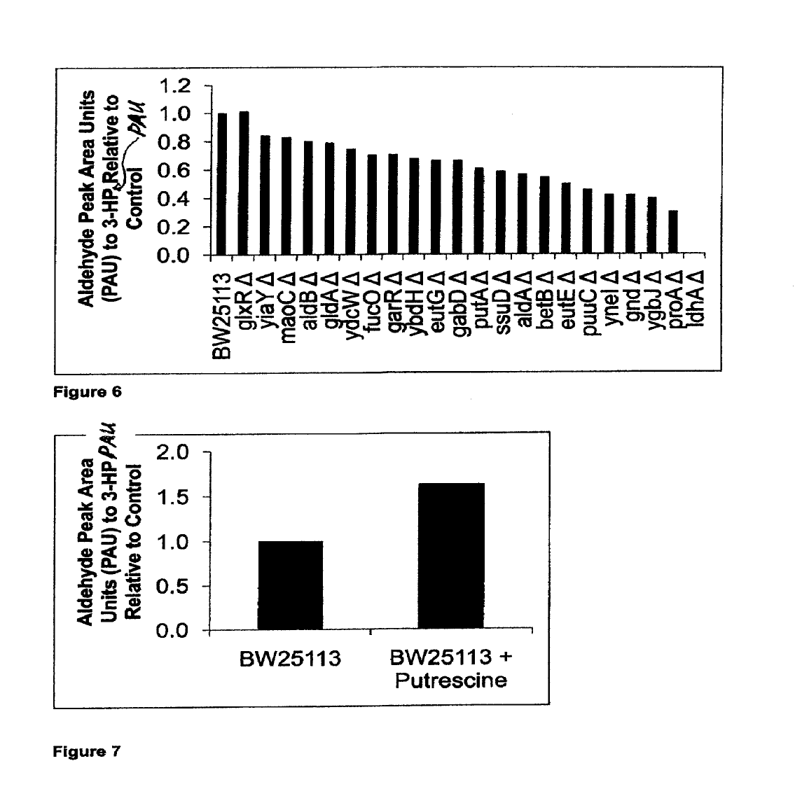

Another 3-HP production pathway is provided in FIG. 5B (FIG. 5A showing the natural mixed fermentation pathways) and explained in this and following paragraphs. This is a 3-HP production pathway that may be used with or independently of other 3-HP production pathways. One possible way to establish this biosynthetic pathway in a recombinant microorganism, one or more nucleic acid sequences encoding an oxaloacetate alpha-decarboxylase (oad-2) enzyme (or respective or related enzyme having such activity) is introduced into a microorganism and expressed. For this and other 3-HP production pathways, enzyme evolution techniques may be applied to enzymes having a desired catalytic role for a structurally similar substrate, so as to obtain an evolved (e.g., mutated) enzyme (and corresponding nucleic acid sequence(s) encoding it), that exhibits the desired catalytic reaction at a desired rate and specificity in a microorganism.

As noted, the above examples of 3-HP production pathways, and particular enzymes (and the nucleic acid sequences encoding them) that are important to complete or improve flux to 3-HP through such pathways, are not meant to be limiting particularly in view of the various known approaches, standard in the art, to achieve desired metabolic conversions. Specific nucleic acid and amino acid sequences corresponding to the enzyme names and activities provided herein (e.g., for 3-HP production), including the claims, are readily found at widely used databases including www.metacyc.org, www.brenda-enzymes.org, and www.ncbi.gov.

D. Discussion of Microorganism Species

The examples below describe specific modifications and evaluations to certain bacterial and yeast microorganisms. The scope of the invention is not meant to be limited to such species, but to be generally applicable to a wide range of suitable microorganisms. As the genomes of various species become known, features of the present invention easily may be applied to an ever-increasing range of suitable microorganisms. Further, given the relatively low cost of genetic sequencing, the genetic sequence of a species of interest may readily be determined to make application of aspects of the present invention more readily obtainable (based on the ease of application of genetic modifications to an organism having a known genomic sequence). More generally, a microorganism used for the present invention may be selected from bacteria, cyanobacteria, filamentous fungi and yeasts.

More particularly, based on the various criteria described herein, suitable microbial hosts for the bio-production of 3-HP that comprise tolerance aspects provided herein generally may include, but are not limited to, any gram negative organisms such as E. coli, Oligotropha carboxidovorans, or Pseudomononas sp.; any gram positive microorganism, for example Bacillus subtilis, Lactobaccilus sp. or Lactococcus sp. a yeast, for example Saccharomyces cerevisiae, Pichia pastoris or Pichia stipitis; and other groups or microbial species. More particularly, suitable microbial hosts for the bio-production of 3-HP generally include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula and Saccharomyces. Hosts that may be particularly of interest include: Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis and Saccharomyces cerevisiae.

Further, in some embodiments, the recombinant microorganism is a gram-negative bacterium. In some embodiments, the recombinant microorganism is selected from the genera Zymomonas, Escherichia, Pseudomonas, Alcaligenes, and Klebsiella, In some embodiments, the recombinant microorganism is selected from the species Escherichia coli, Cupriavidus necator, Oligotropha carboxidovorans, and Pseudomonas putida. In some embodiments, the recombinant microorganism is an E. coli strain.

In some embodiments, the recombinant microorganism is a gram-positive bacterium. In some embodiments, the recombinant microorganism is selected from the genera Clostridium, Salmonella, Rhodococcus, Bacillus, Lactobacillus, Enterococcus, Paenibacillus, Arthrobacter, Corynebacterium, and Brevibacterium. In some embodiments, the recombinant microorganism is selected from the species Bacillus licheniformis, Paenibacillus macerans, Rhodococcus erythropolis, Lactobacillus plantarum, Enterococcus faecium, Enterococcus gallinarium, Enterococcus faecalis, and Bacillus subtilis. In some embodiments, the recombinant microorganism is a B. subtilis strain.

In some embodiments, the recombinant microorganism is a yeast. In some embodiments, the recombinant microorganism is selected from the genera Pichia, Candida, Hansenula and Saccharomyces. In some embodiments, the recombinant microorganism is Saccharomyces cerevisiae.

Species and other phylogenic identifications, above and elsewhere in this application, are according to the classification known to a person skilled in the art of microbiology.

Features as described and claimed herein directed to genetic modifications of aldehyde dehydrogenases, such as to decrease conversion of 3-HP to its aldehydes, may be provided in a microorganism selected from the above listing, or another suitable microorganism, that may also comprise one or more genetic modifications providing increased 3-HP production through natural, introduced, and/or novel 3-HP bio-production pathways. Thus, in some embodiments the microorganism comprises an endogenous 3-HP production pathway (which may, in some such embodiments, be enhanced), whereas in other embodiments the microorganism does not comprise an endogenous 3-HP production pathway, but is provided with one or more nucleic acid sequences encoding polypeptides having enzymatic activity to complete a pathway resulting in production of 3-HP.

E. Other Aspects of Scope of the Invention

Genetic Modifications and Related Definitions

The ability to genetically modify a host cell is essential for the production of any genetically modified, e.g., recombinant microorganism. The mode of gene transfer technology may be by electroporation, conjugation, transduction or natural transformation. A broad range of host conjugative plasmids and drug resistance markers are available. The cloning vectors are tailored to the host organisms based on the nature of antibiotic resistance markers that can function in that host.

For various embodiments of the invention the genetic manipulations to any selected aldehyde dehydrogenases and any of the 3-HP bio-production pathways may be described to include various genetic manipulations, including those directed to change regulation of, and therefore ultimate activity of, an enzyme or enzymatic activity of an enzyme identified in any of the respective pathways. Such genetic modifications may be directed to transcriptional, translational, and post-translational modifications that result in a change of enzyme activity and/or selectivity under selected and/or identified culture conditions and/or to provision of additional nucleic acid sequences (as provided in some of the Examples) such as to increase copy number and/or mutants of an enzyme related to 3-HP production. Specific methodologies and approaches to achieve such genetic modification are well known to one skilled in the art, and include, but are not limited to: increasing expression of an endogenous genetic element; decreasing functionality of a repressor gene; introducing a heterologous genetic element; increasing copy number of a nucleic acid sequence encoding a polypeptide catalyzing an enzymatic conversion step to produce 3-HP; mutating a genetic element to provide a mutated protein to increase specific enzymatic activity; over-expressing; under-expressing; over-expressing a chaperone; knocking out a protease; altering or modifying feedback inhibition; providing an enzyme variant comprising one or more of an impaired binding site for a repressor and/or competitive inhibitor; knocking out a repressor gene; evolution, selection and/or other approaches to improve mRNA stability as well as use of plasmids having an effective copy number and promoters to achieve an effective level of improvement. Random mutagenesis may be practiced to provide genetic modifications that may fall into any of these or other stated approaches. The genetic modifications further broadly fall into additions (including insertions), deletions (such as by a mutation) and substitutions of one or more nucleic acids in a nucleic acid of interest. In various embodiments a genetic modification results in improved enzymatic specific activity and/or turnover number of an enzyme. Without being limited, changes may be measured by one or more of the following: K_{M}; K_{cat}; and K_{avidity}.

In various embodiments, to function more efficiently, a microorganism may comprise one or more gene deletions. For example, in E. coli, the genes encoding the pyruvate kinase (pfkA and pfkB), lactate dehydrogenase (ldhA), phosphate acetyltransferase (pta), pyruvate oxidase (poxB) and pyruvate-formate lyase (pflB) may be deleted. Such gene deletions are summarized at the bottom of FIG. 5B for a particular embodiment, which is not meant to be limiting. Gene deletions may be accomplished by mutational gene deletion approaches, and/or starting with a mutant strain having reduced or no expression of one or more of these enzymes, and/or other methods known to those skilled in the art. Gene deletions may be effectuated by any of a number of known specific methodologies, including but not limited to the RED/ET methods using kits and other reagents sold by Gene Bridges (Gene Bridges GmbH, Dresden, Germany, www.genebridges.com). Further, for 3-HP production, such genetic modifications may be chosen and/or selected for to achieve a higher flux rate through certain basic pathways within the respective 3-HP production pathway and so may affect general cellular metabolism in fundamental and/or major ways. For genetic modifications to reduce or eliminate activity of selected aldhehyde dehdrogenases, gene disruption often is used, although other approaches known to those skilled in the art may also or alternatively be utilized.

As used herein, the term “gene disruption,” or grammatical equivalents thereof (and including “to disrupt enzymatic function,” disruption of enzymatic function,” and the like), is intended to mean a genetic modification to a microorganism that renders the encoded gene product as having a reduced polypeptide activity compared with polypeptide activity in or from a microorganism cell not so modified. The genetic modification can be, for example, deletion of the entire gene, deletion or other modification of a regulatory sequence required for transcription or translation, deletion of a portion of the gene which results in a truncated gene product (e.g., enzyme) or by any of various mutation strategies that reduces activity (including to no detectable activity level) the encoded gene product. A disruption may broadly include a deletion of all or part of the nucleic acid sequence encoding the enzyme, and also includes, but is not limited to other types of genetic modifications, e.g., introduction of stop codons, frame shift mutations, introduction or removal of portions of the gene, and introduction of a degradation signal, those genetic modifications affecting mRNA transcription levels and/or stability, and altering the promoter or repressor upstream of the gene encoding the enzyme.

In some embodiments, a gene disruption is taken to mean any genetic modification to the DNA, mRNA encoded from the DNA, and the amino acid sequence resulting there from that results in reduced polypeptide activity. Many different methods can be used to make a cell having reduced polypeptide activity. For example, a cell can be engineered to have a disrupted regulatory sequence or polypeptide-encoding sequence using common mutagenesis or knock-out technology. See, e.g., Methods in Yeast Genetics (1997 edition), Adams, Gottschling, Kaiser, and Sterns, Cold Spring Harbor Press (1998). One particularly useful method of gene disruption is complete gene deletion because it reduces or eliminates the occurrence of genetic reversions in the genetically modified microorganisms of the invention. Accordingly, a gene disruption of gene whose product is an enzyme thereby disrupts enzymatic function. Alternatively, antisense technology can be used to reduce the activity of a particular polypeptide. For example, a cell can be engineered to contain a cDNA that encodes an antisense molecule that prevents a polypeptide from being translated. The term “antisense molecule” as used herein encompasses any nucleic acid molecule or nucleic acid analog (e.g., peptide nucleic acids) that contains a sequence that corresponds to the coding strand of an endogenous polypeptide. An antisense molecule also can have flanking sequences (e.g., regulatory sequences). Thus, antisense molecules can be ribozymes or antisense oligonucleotides. A ribozyme can have any general structure including, without limitation, hairpin, hammerhead, or axhead structures, provided the molecule cleaves RNA. Further, gene silencing can be used to reduce the activity of a particular polypeptide.

Gene disruptions may be identified that “reduce enzymatic conversion of 3-hydroxypropionic acid (“3-HP”) to an aldehyde of 3-HP,” and one or more such gene disruptions may be introduced into a microorganism host cell to decrease such overall conversion rate under various culture conditions. As used herein, the term “to reduce enzymatic conversion of 3-hydroxypropionic acid (“3-HP”) to an aldehyde of 3-HP” and grammatical equivalents thereof are intended to indicate a reduction in such conversions relative to a control microorganism lacking the genetic modifications shown to provide this result. Also, the term “reduction” or “to reduce” when used in such phrase and its grammatical equivalents are intended to encompass a complete elimination of such conversion(s).

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “microorganism” includes a single microorganism as well as a plurality of microorganisms; and the like.

The term “heterologous DNA,” “heterologous nucleic acid sequence,” and the like as used herein refers to a nucleic acid sequence wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid.

Embodiments of the present invention may result from introduction of an expression vector into a host microorganism, wherein the expression vector contains a nucleic acid sequence coding for an enzyme that is, or is not, normally found in a host microorganism. With reference to the host microorganism's genome prior to the introduction of the heterologous nucleic acid sequence, then, the nucleic acid sequence that codes for the enzyme is heterologous (whether or not the heterologous nucleic acid sequence is introduced into that genome). Also, when the genetic modification of a gene product, i.e., an enzyme, is referred to herein, including the claims, it is understood that the genetic modification is of a nucleic acid sequence, such as or including the gene, that normally encodes the stated gene product, i.e., the enzyme.

Also as used herein, the terms “production” and “bio-production” are used interchangeably when referring to microbial synthesis of 3-HP.

Sequence Listing Free Text

This section is provided to comply with paragraph 36 of Annex C of the PCT Administrative Instructions. Artificial sequences provided in the sequence listing comprise codon-optimized genes, such as mcr (malonyl CoA reductase) provided in a chemically synthesized plasmid in SEQ ID NO:159, the plasmid pHT08 of SEQ ID NO: 160, a chemically synthesized yeast plasmid of SEQ ID NO:166, and its related chemically synthesized plasmid comprising codon optimized mcr as SEQ ID NO:167. Other artificial sequences include primers, plasmids and other constructs. All of these indicated artificial sequences are chemically synthesized at least in part, and thereby are identified as chemically synthesized.

Bio-Production Media

Bio-production media, which is used embodiments of the present invention with recombinant microorganisms, including those having a biosynthetic pathway for 3-HP, must contain suitable carbon substrates for the intended metabolic pathways. Suitable substrates may include, but are not limited to, monosaccharides such as glucose and fructose, oligosaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. Additionally the carbon substrate may also be one-carbon substrates such as carbon dioxide, carbon monoxide, or methanol for which metabolic conversion into key biochemical intermediates has been demonstrated. In addition to one and two carbon substrates methylotrophic organisms are also known to utilize a number of other carbon containing compounds such as methylamine, glucosamine and a variety of amino acids for metabolic activity. For example, methylotrophic yeast are known to utilize the carbon from methylamine to form trehalose or glycerol (Bellion et al., Microb. Growth C1 Compd., [Int. Symp.], 7th (1993), 415-32. Editor(s): Murrell, J. Collin; Kelly, Don P. Publisher: Intercept, Andover, UK). Similarly, various species of Candida will metabolize alanine or oleic acid (Sulter et al., Arch. Microbiol. 153:485-489 (1990)). Hence it is contemplated that the source of carbon utilized in embodiments of the present invention may encompass a wide variety of carbon containing substrates and will only be limited by the choice of organism.

Although it is contemplated that all of the above mentioned carbon substrates and mixtures thereof are suitable for embodiments in the present invention as a carbon source, common carbon substrates used as carbon sources are glucose, fructose, and sucrose, as well as mixtures of any of these sugars. Sucrose may be obtained from feedstocks such as sugar cane, sugar beets, cassava, and sweet sorghum. Glucose and dextrose may be obtained through saccharification of starch based feedstocks including grains such as corn, wheat, rye, barley, and oats.

In addition, fermentable sugars may be obtained from cellulosic and lignocellulosic biomass through processes of pretreatment and saccharification, as described, for example, in US patent application publication number US20070031918A1, which is herein incorporated by reference for its teachings. Biomass refers to any cellulosic or lignocellulosic material and includes materials comprising cellulose, and optionally further comprising hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge from paper manufacture, yard waste, wood and forestry waste. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure. Any such biomass may be used in a bio-production method or system to provide a carbon source.

In addition to an appropriate carbon source, such as selected from one of the above-disclosed types, bio-production media must contain suitable minerals, salts, cofactors, buffers and other components, known to those skilled in the art, suitable for the growth of the cultures and promotion of the enzymatic pathway necessary for 3-HP production.

Finally, in various embodiments the carbon source may be selected to exclude acrylic acid, 1,4-butanediol, as well as other downstream products.

Culture Conditions

Typically cells are grown at a temperature in the range of about 25° C. to about 40° C. in an appropriate medium, as well as up to 70° C. for thermophilic microorganisms. Suitable growth media for embodiments of the present invention are common commercially prepared media such as Luria Bertani (LB) broth, M9 minimal media, Sabouraud Dextrose (SD) broth, Yeast medium (YM) broth (Ymin) yeast synthetic minimal media and minimal media as described herein, such as M9 minimal media. Other defined or synthetic growth media may also be used, and the appropriate medium for growth of the particular microorganism will be known by one skilled in the art of microbiology or bio-production science. In various embodiments a minimal media may be developed and used that does not comprise, or that has a low level of addition (e.g., less than 0.2, or less than one, or less than 0.05 percent) of one or more of yeast extract and/or a complex derivative of a yeast extract, e.g., peptone, tryptone, etc.

Suitable pH ranges for the bio-production are between pH 3.0 to pH 10.0, where pH 6.0 to pH 8.0 is a typical pH range for the initial condition.

However, the actual culture conditions for a particular embodiment are not meant to be limited by the ranges in this section.

Bio-productions may be performed under aerobic, microaerobic, or anaerobic conditions, with or without agitation. The operation of cultures and populations of microorganisms to achieve aerobic, microaerobic and anaerobic conditions are known in the art, and dissolved oxygen levels of a liquid culture comprising a nutrient media and such microorganism populations may be monitored to maintain or confirm a desired aerobic, microaerobic or anaerobic condition.

The amount of 3-HP produced in a bio-production media generally can be determined using a number of methods known in the art, for example, high performance liquid chromatography (HPLC), gas chromatography (GC), or GC/Mass Spectroscopy (MS). Specific HPLC methods for the specific examples are provided herein.

Bio-Production Reactors and Systems:

Any of the recombinant microorganisms as described and/or referred to above may be introduced into an industrial bio-production system where the microorganisms convert a carbon source into 3-HP in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to 3-HP. Industrial bio-production systems and their operation are well-known to those skilled in the arts of chemical engineering and bioprocess engineering. The following paragraphs provide an overview of the methods and aspects of industrial systems that may be used for the bio-production of 3-HP.

In various embodiments, any of a wide range of sugars, including, but not limited to sucrose, glucose, xylose, cellulose or hemicellulose, are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing one or more of the 3-HP biosynthetic pathway alternatives, and the a carbon source may be combined. The carbon source enters the cell and is cataboliized by well-known and common metabolic pathways to yield common metabolic intermediates, including phosphoenolpyruvate (PEP). (See Molecular Biology of the Cell, 3^{rd} Ed., B. Alberts et al. Garland Publishing, New York, 1994, pp. 42-45, 66-74, incorporated by reference for the teachings of basic metabolic catabolic pathways for sugars; Principles of Biochemistry, 3r^{d} Ed., D. L. Nelson & M. M. Cox, Worth Publishers, New York, 2000, pp 527-658, incorporated by reference for the teachings of major metabolic pathways; and Biochemistry, 4^{th} Ed., L. Stryer, W. H. Freeman and Co., New York, 1995, pp. 463-650, also incorporated by reference for the teachings of major metabolic pathways.). The appropriate intermediates are subsequently converted to 3-HP by one or more of the above-disclosed biosynthetic pathways.

Further to types of industrial bio-production, various embodiments of the present invention may employ a batch type of industrial bioreactor. A classical batch bioreactor system is considered “closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate.

A variation on the standard batch system is the Fed-Batch system. Fed-Batch bio-production processes are also suitable when practicing embodiments of the present invention and comprise a typical batch system with the exception that the nutrients, including the substrate, are added in increments as the bio-production progresses. Fed-Batch systems are useful when catabolite repression is apt to inhibit the metabolism of the cells and where it is desirable to have limited amounts of substrate in the media. Measurement of the actual nutrient concentration in Fed-Batch systems may be measured directly, such as by sample analysis at different times, or estimated on the basis of the changes of measurable factors such as pH, dissolved oxygen and the partial pressure of waste gases such as CO₂. Batch and Fed-Batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2^{nd} Ed. J. E. Bailey and D. F. Ollis, McGraw Hill, New York, 1986, herein incorporated by reference for general instruction on bio-production, which as used herein may be aerobic, microaerobic, or anaerobic.

Although embodiments of the present invention may be performed in batch mode, or in fed-batch mode, it is contemplated that the method would be adaptable to continuous bio-production methods. Continuous bio-production is considered an “open” system where a defined bio-production medium is added continuously to a bioreactor and an equal amount of conditioned media is removed simultaneously for processing. Continuous bio-production generally maintains the cultures within a controlled density range where cells are primarily in log phase growth. Two types of continuous bioreactor operation include: 1) Chemostat—where fresh media is fed to the vessel while simultaneously removing an equal rate of the vessel contents. The limitation of this approach is that cells are lost and high cell density generally is not achievable. In fact, typically one can obtain much higher cell density with a fed-batch process. 2) Perfusion culture, which is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bio-production is particularly advantageous for industrial operations because it has less down time associated with draining, cleaning and preparing the equipment for the next bio-production event. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Methods of modulating nutrients and growth factors for continuous bio-production processes as well as techniques for maximizing the rate of product formation are well known in the art of industrial microbiology and a variety of methods are detailed by Brock, supra.

It is contemplated that embodiments of the present invention may be practiced in either batch, fed-batch or continuous processes and that any known mode of bio-production would be suitable. Additionally, it is contemplated that cells may be immobilized on an inert scaffold as whole cell catalysts and subjected to suitable bio-production conditions for 3-HP production. Thus, embodiments used in such processes, and in bio-production systems using these processes, include a population of genetically modified microorganisms of the present invention, and a culture system comprising such population in a media comprising nutrients for the population.

The following published resources are incorporated by reference herein for their respective teachings to indicate the level of skill in these relevant arts, and as needed to support a disclosure that teaches how to make and use methods of industrial bio-production of 3-HP from sugar sources, and also industrial systems that may be used to achieve such conversion with any of the recombinant microorganisms of the present invention (Biochemical Engineering Fundamentals, 2^{nd} Ed. J. E. Bailey and D. F. Ollis, McGraw Hill, New York, 1986, entire book for purposes indicated and Chapter 9, pages 533-657 in particular for biological reactor design; Unit Operations of Chemical Engineering, 5^{th} Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings).

Also, the scope of the present invention is not meant to be limited to the exact sequences provided herein. It is appreciated that a range of modifications to nucleic acid and to amino acid sequences may be made and still provide a desired functionality, such as a desired enzymatic activity and specificity. The following discussion is provided describe ranges of variation that may be practiced and still remain within the scope of the present invention.

It has long been recognized in the art that some amino acids in amino acid sequences can be varied without significant effect on the structure or function of proteins. Variants included can constitute deletions, insertions, inversions, repeats, and type substitutions so long as the indicated enzyme activity is not significantly adversely affected. Guidance concerning which amino acid changes are likely to be phenotypically silent can be found, inter alia, in Bowie, J. U., et Al., “Deciphering the Message in Protein Sequences: Tolerance to Amino Acid Substitutions,” Science 247:1306-1310 (1990). This reference is incorporated by reference for such teachings, which are, however, also generally known to those skilled in the art.

In various embodiments polypeptides obtained by the expression of the polynucleotide molecules of the present invention may have at least approximately 50%, 60%, 70%, 80%, 90%, 95%, 96%, 97%, 98%, 99% or 100% identity to one or more amino acid sequences encoded by the genes and/or nucleic acid sequences described herein for the 3-HP biosynthesis pathways. A truncated respective polypeptide has at least about 90% of the full length of a polypeptide encoded by a nucleic acid sequence encoding the respective native enzyme, and more particularly at least 95% of the full length of a polypeptide encoded by a nucleic acid sequence encoding the respective native enzyme. By a polypeptide having an amino acid sequence at least, for example, 95% “identical” to a reference amino acid sequence of a polypeptide is intended that the amino acid sequence of the claimed polypeptide is identical to the reference sequence except that the claimed polypeptide sequence can include up to five amino acid alterations per each 100 amino acids of the reference amino acid of the polypeptide. In other words, to obtain a polypeptide having an amino acid sequence at least 95% identical to a reference amino acid sequence, up to 5% of the amino acid residues in the reference sequence can be deleted or substituted with another amino acid, or a number of amino acids up to 5% of the total amino acid residues in the reference sequence can be inserted into the reference sequence. These alterations of the reference sequence can occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 50%, 60%, 70%, 80%, 85%, 90%, 92%, 95%, 96%, 97%, 98% or 99% identical to any reference amino acid sequence of any polypeptide described herein (which may correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set such that the percentage of identity is calculated over the full length of the reference amino acid sequence and that gaps in identity of up to 5% of the total number of amino acid residues in the reference sequence are allowed.

For example, in a specific embodiment the identity between a reference sequence (query sequence, i.e., a sequence of the present invention) and a subject sequence, also referred to as a global sequence alignment, may be determined using the FASTDB computer program based on the algorithm of Brutlag et al. (Comp. App. Biosci. 6:237-245 (1990)). Preferred parameters for a particular embodiment in which identity is narrowly construed, used in a FASTDB amino acid alignment, are: Scoring Scheme=PAM (Percent Accepted Mutations) 0, k-tuple=2, Mismatch Penalty-1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are lateral to the N- and C-terminal of the subject sequence, which are not matched (i.e., aligned) with a corresponding subject residue, as a percent of the total bases of the query sequence. A determination of whether a residue is matched (i.e., aligned) is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched (i.e., aligned) with the query sequence, are considered for the purposes of manually adjusting the percent identity score. That is, only query residue positions outside the farthest N- and C-terminal residues of the subject sequence are considered for this manual correction. For example, a 90 amino acid residue subject sequence is aligned with a 100 residue query sequence to determine percent identity. The deletion occurs at the N-terminus of the subject sequence and therefore, the FASTDB alignment does not show a matching (i.e., alignment) of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched (i.e., aligned) with the query. In this case the percent identity calculated by FASTDB is not manually corrected. Once again, only residue positions outside the N- and C-terminal ends of the subject sequence, as displayed in the FASTDB alignment, which are not matched (i.e., aligned) with the query sequence are manually corrected for.

Also as used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. “Homology”, with respect to polypeptides (i.e., amino acids), may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions, i.e. those that substitute a given amino acid in a polypeptide by another amino acid of similar characteristics. Typically seen as conservative substitutions are the following replacements: replacements of an aliphatic amino acid such as Ala, Val, Leu and Ile with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue. A polypeptide sequence (i.e., amino acid sequence) or a polynucleotide sequence comprising at least 50% homology to another amino acid sequence or another nucleotide sequence respectively has a homology of 50% or greater than 50%, e.g., 60%, 70%, 80%, 90% or 100%.

The above descriptions and methods for sequence identity and homology are intended to be exemplary and it is recognized that these concepts are well-understood in the art. Further, it is appreciated that nucleic acid sequences may be varied and still encode an enzyme or other polypeptide exhibiting a desired functionality, and such variations are within the scope of the present invention. Nucleic acid sequences that encode polypeptides that provide the indicated functions for 3-HP increased production are considered within the scope of the present invention. These may be further defined by the stringency of hybridization, described below, but this is not meant to be limiting when a function of an encoded polypeptide matches a specified 3-HP biosynthesis pathway enzyme activity.

Further to nucleic acid sequences, “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1M, more usually less than about 500 mM and less than about 200 mM. Hybridization temperatures can be as low as 5° C., but are typically greater than 22° C., more typically greater than about 30° C., and often are in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook and Russell and Anderson “Nucleic Acid Hybridization” 1^{st} Ed., BIOS Scientific Publishers Limited (1999), which is hereby incorporated by reference for hybridization protocols. “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

In one aspect of the invention the identity values in the preceding paragraphs are determined using the parameter set described above for the FASTDB software program. It is recognized that identity may be determined alternatively with other recognized parameter sets, and that different software programs (e.g., Bestfit vs. BLASTp) are expected to provide different results. Thus, identity can be determined in various ways. Further, for all specifically recited sequences herein it is understood that conservatively modified variants thereof are intended to be included within the invention.

In some embodiments, the invention contemplates a genetically modified (e.g., recombinant) microorganism comprising a heterologous nucleic acid sequence that encodes a polypeptide that is an identified enzymatic functional variant of any of the enzymes of any 3-HP production pathway, wherein the polypeptide has enzymatic activity and specificity effective to perform the enzymatic reaction of the respective 3-HP production enzyme, so that the recombinant microorganism exhibits greater 3-HP production than an appropriate control microorganism lacking such nucleic acid sequence. Relevant methods of the invention also are intended to be directed to identified enzymatic functional variants and the nucleic acid sequences that encode them.

The term “identified enzymatic functional variant” means a polypeptide that is determined to possess an enzymatic activity and specificity of an enzyme of interest but which has an amino acid sequence different from such enzyme of interest. A corresponding “variant nucleic acid sequence” may be constructed that is determined to encode such an identified enzymatic functional variant. For a particular purpose, such as increased production of 3-HP via genetic modification to increase enzymatic conversion at one or more of the enzymatic conversion steps of a 3-HP pathways in a microorganism, one or more genetic modifications may be made to provide one or more heterologous nucleic acid sequence(s) that encode one or more identified 3-HP production enzymatic functional variant(s). That is, each such nucleic acid sequence encodes a polypeptide that is not exactly the known polypeptide of an enzyme of that 3-HP pathway, but which nonetheless is shown to exhibit enzymatic activity of such enzyme. Such nucleic acid sequence, and the polypeptide it encodes, may not fall within a specified limit of homology or identity yet by its provision in a cell nonetheless provide for a desired enzymatic activity and specificity. The ability to obtain such variant nucleic acid sequences and identified enzymatic functional variants is supported by recent advances in the states of the art in bioinformatics and protein engineering and design, including advances in computational, predictive and high-throughput methodologies.

It is understood that the steps described herein and also exemplified in the non-limiting examples below comprise steps to make a genetic modification, and steps to identify a genetic modification such as to reduce conversion of 3-HP to its aldehydes and to improve 3-HP production in a microorganism and/or in a microorganism culture or culture system. Also, the genetic modifications so obtained and/or identified comprise means to make a microorganism exhibiting these features.

Having so described multiple aspects of the present invention and provided examples below, and in view of the above paragraphs, it is appreciated that various non-limiting aspects of the present invention may include, but are not limited to, the following embodiments.

In some embodiments, the invention contemplates a method of making a genetically modified microorganism comprising: a) providing to a selected microorganism at least one genetic modification of a 3-hydroxypropionic acid (“3-HP”) production pathway to increase microbial synthesis of 3-HP above the rate of a control microorganism lacking the at least one genetic modification; and b) providing to the selected microorganism at least one genetic modification of two or more aldehyde dehydrogenases. In some embodiments, the 3-HP production pathway is introduced into the selected microorganism. Some embodiments comprise providing a nucleic acid sequence encoding one of a malonyl Co-A reductase, a 3-hydroxyacid reductase, a 3-hydroxyacid reductase having at least 85% identity with the ydfG of E. coli, a nucleic acid sequence encoding a β-alanine aminotransferase, a nucleic acid sequence encoding an alanine-2,3-aminotransferase, an oxaloacetate α-decarboxylase, a glycerol dehydratase, a 3-phoshpoglycerate phosphatase, a glycerate dehydratase, and a β-alanine aminotransferase. In some embodiments, the control microorganism does not produce 3-HP. Some embodiments comprise providing at least one said genetic modification to each of at least three aldehyde dehydrogenases. In some embodiments, the aldehyde dehydrogenase genetic modifications are to aldA (SEQ ID NO:001), aldB (SEQ ID NO:002), puuC (SEQ ID NO:016). Some embodiments comprise providing an additional genetic modification of an additional aldehyde dehydrogenase. In some embodiments, the additional genetic modification comprises at least one genetic modification of a nucleic acid sequence encoding an aldehyde dehydrogenase enzyme, wherein the additional genetic modification disrupts enzymatic function of an additional aldehyde dehydrogenase. Some embodiments comprise providing at least one said genetic modification to each of at least four, or each of at least 5, aldehyde dehydrogenases. Some embodiments comprise disruptions of aldA (SEQ ID NO:001), aldB (SEQ ID NO:002), puuC (SEQ ID NO:016), and usg (SEQ ID NO:120). Some embodiments comprise disrupting an enzymatic function of one or more aldehyde dehydrogenases. In some embodiments, the disrupting of enzymatic function of one or more aldehyde dehydrogenases reduces enzymatic conversion of 3-HP to an aldehyde of 3-HP. Some embodiments comprise disrupting one of aldA (SEQ ID NO:001), aldB (SEQ ID NO:002), puuC (SEQ ID NO:016), and usg (SEQ ID NO:120). Some embodiments comprise disrupting aldA (SEQ ID NO:001) and aldB (SEQ ID NO:002); or aldA (SEQ ID NO:001) and puuC (SEQ ID NO:016); or aldA (SEQ ID NO:001) and usg (SEQ ID NO:120); or aldB (SEQ ID NO:002) and puuC (SEQ ID NO:016); or aldB (SEQ ID NO:002) and usg (SEQ ID NO:120); or puuC (SEQ ID NO:016) and usg (SEQ ID NO:120). Some embodiments comprise disrupting aldA (SEQ ID NO:001), aldB (SEQ ID NO:002), and puuC (SEQ ID NO:016); or aldA (SEQ ID NO:001), aldB (SEQ ID NO:002), and usg (SEQ ID NO:120); or aldA (SEQ ID NO:001), aldB (SEQ ID NO:002), puuC (SEQ ID NO:016), and usg (SEQ ID NO:120). In some embodiments, the at least one genetic modification of an aldehyde dehydrogenase comprises at least one genetic modification of a nucleic acid sequence encoding an enzyme having aldehyde dehydrogenase activity. Some embodiments comprise selecting the aldehyde dehydrogenase from Table 1. Some embodiments additionally comprise disrupting a nucleic acid sequence encoding lactate dehydrogenase. In some embodiments, the selected microorganism comprises a disruption of a nucleic acid sequence encoding lactate dehydrogenase. In some embodiments, the lactate dehydrogenase comprises ldhA (SEQ ID NO:012).

In some embodiments, the invention contemplates a method of making a genetically modified microorganism comprising introducing at least one genetic modification into a microorganism to decrease its enzymatic conversion of 3-hydroxypropionic acid (“3-HP”) to an aldehyde of 3-HP, wherein the genetically modified microorganism synthesizes 3-HP. In some embodiments, the at least one genetic modification decreases 3-HP metabolism to the aldehyde in the genetically modified microorganism below the 3-HP metabolism of a control microorganism lacking the genetic modification. Some embodiments comprise introducing at least two, at least three, at least four, or at least five said genetic modifications. Some embodiments additionally comprise providing in the genetically modified microorganism at least one genetic modification to increase 3-HP production. In some embodiments, the genetic modification(s) to decrease metabolism comprises disruption of at least one nucleic acid sequence that encodes an aldehyde dehydrogenase. In some embodiments, the aldehyde dehydrogenase is selected from Table 1. In some embodiments, each of the genetic modifications comprises a disruption of a nucleic acid sequence encoding an enzyme that is within a 50, 60, 70, 80, 90, or 95 percent homology of one of the aldehyde dehydrogenase amino acid sequences of Table 1. Some embodiments comprise selecting for said introduced genetic modification a nucleic acid sequence encoding an enzyme that is within a 50, 60, 70, 80, 90, or 95 percent homology of one of the aldehyde dehydrogenase amino acid sequences of Table 1, and evaluating a disruption of that nucleic acid sequence for its effect on said decrease of enzymatic conversion of 3-HP to an aldehyde of 3-HP. Some embodiments comprise providing in the microorganism at least one heterologous nucleic acid sequence encoding an enzyme in a 3-HP production pathway. Some embodiments comprise providing a nucleic acid sequence encoding one of malonyl Co-A reductase, a 3-hydroxyacid reductase, a 3-hydroxyacid reductase having at least 85% identity with the ydfG of E. coli, a β-alanine aminotransferase, an alanine-2,3-aminotransferase, an oxaloacetate α-decarboxylase, a glycerol dehydratase, a 3-phoshpoglycerate phosphatase, a glycerate dehydratase, and a β-alanine aminotransferase. In some embodiments, the invention contemplates a method comprising: a) introducing to a selected microorganism at least one genetic modification of a nucleic acid sequence encoding an enzyme that is within a 50, 60, 70, 80, 90, or 95 percent homology of one of the aldehyde dehydrogenase amino acid sequences of Table 1; and b) evaluating the microorganism of step a for a difference in conversion of 3-hydroxypropionic acid (“3-HP”) to an aldehyde of 3-HP compared to a control microorganism lacking the at least one genetic modification. Some embodiments comprise disrupting the nucleic acid sequence. In some embodiments, the nucleic acid sequence encodes an enzyme having aldehyde dehydrogenase activity. In some embodiments, the evaluating is made under aerobic conditions, anaerobic conditions, or microaerobic conditions. In some embodiments, the selected microorganism produces 3-HP. In some embodiments, the method additionally comprises providing one or more said genetic modifications to a second microorganism that produces 3-HP. Some embodiments comprise providing in the second microorganism at least one heterologous nucleic acid sequence encoding an enzyme along a 3-HP production pathway, effective to increase 3-HP production in the second microorganism. Some embodiments comprise providing a nucleic acid sequence encoding one of malonyl Co-A reductase, a 3-hydroxyacid reductase, a 3-hydroxyacid reductase having at least 85% identity with the ydfG of E. coli, a β-alanine aminotransferase, an alanine-2,3-aminotransferase, an oxaloacetate α-decarboxylase, a glycerol dehydratase, a 3-phoshpoglycerate phosphatase, a glycerate dehydratase, and a β-alanine aminotransferase. In some embodiments, the invention contemplates a method of making a microorganism comprising one or more genetic modifications directed to reducing conversion of 3-hydroxypropionic acid (“3-HP”) to aldehydes comprising: a) introducing into a selected microorganism at least one genetic modification of an aldehyde dehydrogenase; b) evaluating the microorganism of step a for decreased conversion of 3-HP to an aldehyde of 3-HP; and c) optionally repeating steps a and b iteratively to obtain a microorganism comprising multiple genetic modifications directed to reducing conversion of 3-HP to aldehydes. Some embodiments additionally comprise providing a nucleic acid sequence that encodes an enzyme, the expression of which increases production of 3-HP along a metabolic path in the microorganism increases comprising the enzyme. In some embodiments, the evaluating is made under aerobic conditions, anaerobic conditions, or microaerobic conditions.

In some embodiments, the invention contemplates a genetically modified microorganism made by a method of the instant invention.

In some embodiments, the invention contemplates a genetically modified microorganism comprising: a) at least one genetic modification to produce 3-hydroxypropionic acid (“3-HP”); and b) at least one genetic modification of at least two aldehyde dehydrogenases effective to decrease each said aldehyde dehydrogenase's respective enzymatic activity and effective to decrease metabolism of 3-HP to any aldehydes of 3-HP, as compared to the metabolism of a control microorganism lacking the at least two genetic modifications of the aldehyde dehydrogenases. Some embodiments comprise at least one said genetic modification to each of at least three aldehyde dehydrogenases. In some embodiments, the aldehyde dehydrogenase genetic modifications are to aldA (SEQ ID NO:001), aldB (SEQ ID NO:002), and puuC (SEQ ID NO:016). Some embodiments additionally comprise at least one genetic modification of an additional aldehyde dehydrogenase. In some embodiments, the genetically modified microorganism additionally comprises a genetic modification of ydfG (SEQ ID NO:168) or usg (SEQ 1D NO:120). Some embodiments comprise at least one said genetic modification to each of at least four aldehyde dehydrogenases. In some embodiments, the at least one genetic modification comprises a disruption of enzymatic function of at least one aldehyde dehydrogenase. In some embodiments, one said genetic modification comprises a disruption of one of aldA (SEQ ID NO:001), aldB (SEQ ID NO:002), puuC (SEQ ID NO:016), and usg (SEQ ID NO:120). In some embodiments, one said genetic modification comprises a disruption of aldA (SEQ ID NO:001) and aldB (SEQ ID NO:002), or aldA (SEQ ID NO:001) and puuC (SEQ ID NO:016), or aldA (SEQ ID NO:001) and usg (SEQ ID NO:120), or aldB (SEQ ID NO:002) and puuC (SEQ ID NO:016), or aldB (SEQ ID NO:002) and usg (SEQ ID NO:120), or puuC (SEQ ID NO:016) and usg (SEQ ID NO:120), or aldA (SEQ ID NO:001), aldB (SEQ ID NO:002), and puuC (SEQ ID NO:016), or aldA (SEQ ID NO:001), aldB (SEQ ID NO:002), and usg (SEQ ID NO:120), or aldA (SEQ ID NO:001), aldB (SEQ ID NO:002), puuC (SEQ ID NO:016), and usg (SEQ ID NO:120). In some embodiments, the at least one genetic modification comprises a deletion of one or more genes encoding the at least one aldehyde dehydrogenase.

In some embodiments, the invention contemplates a genetically modified microorganism comprising at least one genetic modification of each of two or more aldehyde dehydrogenases, said aldehyde dehydrogenases capable of converting 3-hydroxypropionic acid (“3-HP”) to any of its aldehyde metabolites. In some embodiments, the genetic modifications disrupt enzymatic function of the two or more, or of three of more, aldehyde dehydrogenases. In some embodiments, the aldehyde dehydrogenase genetic modifications comprise modifications to puuC, aldA and aldB. In some embodiments, the genetically modified microorganism comprises an additional aldehyde dehydrogenase genetic modification. In some embodiments, the genetic modifications disrupt enzymatic function of four or more aldehyde dehydrogenases. In some embodiments, the at least one genetic modification to produce 3-HP increases microbial synthesis of 3-HP above a rate or titer of a control microorganism lacking the at least one genetic modification to produce 3-HP. In some embodiments, the at least one genetic modification to produce 3-HP comprises providing a nucleic acid sequence that encodes an enzyme of a 3-HP production pathway. In some embodiments, the enzyme is one of malonyl Co-A reductase, a 3-hydroxyacid reductase, a 3-hydroxyacid reductase having at least 85% identity with the ydfG of E. coli, a β-alanine aminotransferase, an alanine-2,3-aminotransferase, an oxaloacetate α-decarboxylase, a glycerol dehydratase, a 3-phoshpoglycerate phosphatase, a glycerate dehydratase, and a β-alanine aminotransferase. In some embodiments, at least one genetic modification, to the aldehyde dehydrogenase comprises a gene deletion.

In some embodiments, the invention contemplates a genetically modified microorganism comprising at least one genetic modification of each of at least two aldehyde dehydrogenases effective to decrease microbial enzymatic conversion of 3-hydroxypropionic acid (“3-HP”) to an aldehyde of 3-HP as compared to the enzymatic conversion of a control microorganism lacking the genetic modifications. In some embodiments, the genetically modified microorganism comprises at least one said genetic modification to each of at least three aldehyde dehydrogenases. In some embodiments, the aldehyde dehydrogenase genetic modifications comprise modifications to puuC, aldA and aldB. In some embodiments, the genetically modified microorganism further comprises a genetic modification to an additional aldehyde dehydrogenase. In some embodiments, the genetically modified microorganism comprises at least one said genetic modification to each of at least four aldehyde dehydrogenases. In some embodiments, at least one said genetic modification is a gene disruption or deletion. In some embodiments, each said aldehyde dehydrogenase comprises an amino acid sequence comprising at least 50%, 60%, 70%, 80%, 85%, 90%, 92%, 95%, 96%, 97%, 98% or 99% sequence identity to an amino acid sequence selected from the group consisting of aldA (SEQ ID NO:001), aldB (SEQ ID NO:002), puuC (SEQ ID NO:016), and usg (SEQ ID NO:120). In some embodiments, each said aldehyde dehydrogenase is selected from the group consisting of aldA (SEQ ID NO:001), aldB (SEQ ID NO:002), puuC (SEQ ID NO:016), and usg (SEQ ID NO:120). In some embodiments, the nucleic acid sequence having the genetic modification has greater than 70%, greater than 75%, greater than 80%, greater than 85%, greater than 90%, greater than 95% sequence identity to an aldehyde dehydrogenase selected from the group consisting of aldA (SEQ ID NO:001), aldB (SEQ ID NO:002), puuC (SEQ ID NO:016), and usg (SEQ ID NO:120). In some embodiments, the aldehyde is selected from the group consisting of 3-hydroxypropionaldehyde (“3-HPA”), malonate semialdehyde (“MSA”), malonate, and malonate di-aldehyde. In some embodiments, said aldehyde dehydrogenase genetic modifications are effective to decrease enzymatic conversions of 3-HP to its aldehydes by at least about 5 percent, at least about 10 percent, at least about 20 percent, at least about 30 percent, or at least about 50 percent above said enzymatic conversions of a control microorganism lacking said aldehyde dehydrogenase genetic modifications. In some embodiments, control microorganism does not produce 3-HP. In some embodiments, does produce 3-HP. In some embodiments, the genetically modified microorganism additionally comprises a disruption of a nucleic acid sequence encoding lactate dehydrogenase. In some embodiments, the selected microorganism comprises a disruption of a nucleic acid sequence encoding lactate dehydrogenase. In some embodiments, SEQ ID NO:012 is the disrupted lactate dehydrogenase. In some embodiments, the genetically modified microorganism is a gram-negative bacterium. In some embodiments, the genetically modified microorganism is selected from the genera: Zymomonas, Escherichia, Pseudomonas, Alcaligenes, Salmonella, Shigella, Burkholderia, Oligotropha, and Klebsiella. In some embodiments, the genetically modified microorganism is selected from the species: Escherichia coli, Cupriavidus necator, Oligotropha carboxidovorans, and Pseudomonas putida. In some embodiments, the genetically modified microorganism is an E. coli strain. In some embodiments, the genetically modified microorganism is a gram-positive bacterium. In some embodiments, the genetically modified microorganism is selected from the genera: Clostridium, Rhodococcus, Bacillus, Lactobacillus, Enterococcus, Paenibacillus, Arthrobacter, Corynebacterium, and Brevibacterium. In some embodiments, the genetically modified microorganism is selected from the species: Bacillus licheniformis, Paenibacillus macerans, Rhodococcus erythropolis, Lactobacillus plantarum, Enterococcus faecium, Enterococcus gallinarium, Enterococcus faecalis, and Bacillus subtilis. In some embodiments, the genetically modified microorganism is a B. subtilis strain. In some embodiments, the genetically modified microorganism is a fungus or a yeast. In some embodiments, the genetically modified microorganism is selected from the genera: Pichia, Candida, Hansenula and Saccharomyces. In some embodiments, the genetically modified microorganism is Saccharomyces cerevisiae. In some embodiments, the genetic modification of the aldehyde dehydrogenase exhibits a difference from a control microorganism lacking said genetic modification in conversion of 3-HP to one of its aldehydes under aerobic culture conditions. In some embodiments, the genetic modification of the aldehyde dehydrogenase exhibits a difference from a control microorganism lacking said genetic modification in conversion of 3-HP to one of its aldehydes under anaerobic culture conditions. In some embodiments, the genetic modification of the aldehyde dehydrogenase exhibits a difference from a control microorganism lacking said genetic modification in conversion of 3-HP to one of its aldehydes under microaerobic culture conditions.

In some embodiments, the invention contemplates a culture system comprising: a) a population of a genetically modified microorganism as described herein; and b) a media comprising nutrients for the population.

Also, it is recognized for some embodiments that the enzyme 3-hydroxyacid dehydrogenase, such as that enzyme encoded by ydfG in E. coli (SEQ ID NO:168 for nucleic acid sequence, SEQ ID NO:169 for encoded amino acid sequence of the enzyme, www.ecocyc.org), may be genetically modified in various manners in a microorganism being modified for production of 3-HP. One group of such genetic modifications comprise disruptions, including deletions, to decrease enzymatic conversion of 3-HP to its aldehydes. In other embodiments, genetic modifications may be made to increase 3-hydroxyacid dehydrogenase enzymatic activity in order to increase production of 3-HP from malonate semialdehyde, which reaction is known.

In some embodiments, the invention contemplates a recombinant microorganism comprising at least one genetic modification effective to decrease enzymatic activity of an aldehyde dehydrogenase that is effective to decrease metabolism of 3-HP to any aldehydes of 3-HP, in some embodiments also comprising at least one genetic modification effective to increase 3-HP production, wherein the increased level of 3-HP production is greater than the level of 3-HP production in the wild-type microorganism. In some embodiments, the wild-type microorganism produces 3-HP. In some embodiments, the wild-type microorganism does not produce 3-HP. In some embodiments, the recombinant microorganism comprises at least one vector, such as at least one plasmid, wherein the at least one vector comprises at least one heterologous nucleic acid molecule.

In some embodiments of the invention, the at least one genetic modification effective to increase 3-HP production increased 3-HP production above the 3-HP production of a control microorganism by about 5%, 10%, or 20%. In some embodiments, the 3-HP production of the genetically modified microorganism is increased above the 3-HP production of a control microorganism by about 30%, 40%, 50%, 60%, 80%, or 100%.

Also, in various independent groupings of embodiments one or more aldehyde dehydrogenase genetic modifications, such as disruptions, may be selected from the list of Table 1 (such as for providing one or more aldehyde dehydrogenase gene deletions to a selected microorganism), however excluding aldA and its homologues, aldB and its homologues, betB and its homologues, eutE and its homologues, eutG and its homologues, fucO and its homologues, gabD and its homologues, garR and its homologues, gldA and its homologues, glxR and its homologues, gnd and its homologues, ldhA and its homologues, maoC and its homologues, proA and its homologues, putA and its homologues, puuC and its homologues, sad and its homologues, ssuD and its homologues, ybdH and its homologues, ydcW and its homologues, ygbJ and its homologues, yiaY and its homologues, or excluding two or more, or three or more, of such genes and their homologues from such smaller list, or sub-list. For example, a microorganism may be genetically modified to comprise gene deletions of puuC, aldA, aldB and another gene deletion selected from Table 1 however, for this embodiment, excluding ydcW, so the fourth gene deletion could comprise any of the genes of Table 1, and their respective homologues (particularly where these are identified to convert 3-HP to one of its aldehydes), other than ydcW and the already selected puuC, aldA, and aldB gene deletions. In other independent groupings of embodiments, the various sub-lists developed from the list of Table 1 exclude one or more of the above-indicated genes but not their homologues, or, alternatively, one or more of the above-indicated genes and only their respective homologues identified and evaluated to have the capability to convert 3-HP to one of its aldehydes. The following paragraphs disclose more particular embodiments.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, Seq. and ID NO. 044.

In some embodiments, the disruption is a disruption of one or more of the peptides of Seq. ID NO. 023, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO, 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO, 032, Seq. ID NO. 033, Seq. ID NO. 034, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, and Seq. ID NO. 042.

In some embodiments, the disruption is a disruption of one or more of the peptides of Seq. ID NO. 024, Seq. ID NO. 025, Seq. ID NO. 026, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 029, Seq. ID NO. 030, Seq. ID NO. 031, Seq. ID NO. 032, Seq. ID NO. 033, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 025, Seq. ID NO. 026, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 025, Seq. ID NO. 026, Seq. ID NO. 027, Seq. ID NO. 028, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 026, Seq. ID NO. 027, Seq. ID NO. 028, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 026, Seq. ID NO. 027, Seq. ID NO. 028, Seq. ID NO. 029, Seq. ID NO. 030, Seq. ID NO. 031, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 027, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 028, Seq. ID NO. 029, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 031, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 032, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 032, Seq. ID NO. 033, Seq. ID NO. 034, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 032, Seq. ID NO. 033, Seq. ID NO. 034, Seq. ID NO. 035, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 033, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 033, Seq. ID NO. 034, Seq. ID NO. 036, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 033, Seq. ID NO. 034, Seq. ID NO. 035, Seq. ID NO. 036, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 033, Seq. ID NO. 034, Seq. ID NO. 035, Seq. ID NO. 036, Seq. ID NO. 037, Seq. ID NO. 038, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 035, Seq. ID NO. 037, Seq. ID NO. 038, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 035, Seq. ID NO. 036, Seq. ID NO. 037, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 035, Seq. ID NO. 036, Seq. ID NO. 037, Seq. ID NO. 038, Seq. ID NO. 039, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 035, Seq. ID NO. 036, Seq. ID NO. 037, Seq. ID NO. 038, Seq. ID NO. 039, Seq. ID NO. 040, Seq. ID NO. 041, Seq. ID NO. 042, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 037, Seq. ID NO. 039, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 037, Seq. ID NO. 038, Seq. ID NO. 039, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 037, Seq. ID NO. 038, Seq. ID NO. 039, Seq. ID NO. 040, Seq. ID NO. 041, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 038, Seq. ID NO. 040, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 038, Seq. ID NO. 039, Seq. ID NO. 041, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO, 032, Seq. ID NO. 033, Seq. ID NO. 034, Seq. ID NO. 035, Seq. ID NO. 036, Seq. ID NO. 038, Seq. ID NO. 039, Seq. ID NO. 040, Seq. ID NO. 042, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 038, Seq. ID NO. 039, Seq. ID NO. 040, Seq. ID NO. 041, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 038, Seq. ID NO. 039, Seq. ID NO. 040, Seq. ID NO. 041, Seq. ID NO. 042, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 040, Seq. ID NO. 041, Seq. ID NO. 043, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 040, Seq. ID NO. 041, Seq. ID NO. 042, and Seq. ID NO. 043.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 041, Seq. ID NO. 042, and Seq. ID NO. 044.

In some embodiments, the disruption is a disruption of one or more of the peptides of Seq. ID NO. 023, Seq. ID NO. 024, Seq. ID NO. 025, Seq. ID NO. 026, Seq. ID NO. 027, Seq. ID NO. 028, Seq. ID NO. 029, Seq. ID NO. 030, Seq. ID NO. 031, Seq. ID NO. 032, Seq. ID NO. 033, Seq. ID NO. 034, Seq. ID NO. 035, Seq. ID NO. 036, Seq. ID NO. 037, Seq. ID NO. 038, Seq. ID NO. 039, Seq. ID NO. 040, Seq. ID NO. 041, and Seq. ID NO. 043.

Also, in various embodiments the production of 3-HP by a genetically modified microorganism of the present invention, under standard growth conditions, may produce 3-HP at different rates in different phases of growth, and may be cultured to first increase biomass and later produce 3-HP during a period of substantially lower biomass formation rates.

It is noted that the information in the figures, FIGS. 1-11, and in the tables, Tables 1-5, are incorporated into this section of the application for support of the various embodiments of the invention.

The practice of the present invention will employ, unless otherwise indicated, conventional techniques of the biosynthetic industry and the like, which are within the skill of the art. Such techniques are fully explained in the literature and exemplary methods are provided below.

Also, while steps of the example involve use of plasmids, other vectors known in the art may be used instead. These include cosmids, viruses (e.g., bacteriophage, animal viruses, plant viruses), and artificial chromosomes (e.g., yeast artificial chromosomes (YAC) and bacteria artificial chromosomes (BAC)).

Before the specific examples of the invention are described in detail, it is to be understood that, unless otherwise indicated, the present invention is not limited to particular sequences, expression vectors, enzymes, host microorganisms, compositions, processes or systems, or combinations of these, as such may vary. It is also to be understood that the terminology used herein is for purposes of describing particular embodiments only, and is not intended to be limiting.

Also, and more generally, in accordance with disclosures, discussions, examples and embodiments herein, there may be employed conventional molecular biology, cellular biology, microbiology, and recombinant DNA techniques within the skill of the art. Such techniques are explained fully in the literature. (See, e.g., Sambrook and Russell, Molecular Cloning: A Laboratory Manual, Third Edition 2001 (volumes 1-3), Cold Spring Harbor Laboratory Press, Cold Spring Harbor, N.Y.; Animal Cell Culture, R. I. Freshney, ed., 1986). These published resources are incorporated by reference herein for their respective teachings of standard laboratory methods found therein. Further, all patents, patent applications, patent publications, and other publications referenced herein (collectively, “published resource(s)”) are hereby incorporated by reference in this application. Such incorporation, at a minimum, is for the specific teaching and/or other purpose that may be noted when citing the reference herein. If a specific teaching and/or other purpose is not so noted, then the published resource is specifically incorporated for the teaching(s) indicated by one or more of the title, abstract, and/or summary of the reference. If no such specifically identified teaching and/or other purpose may be so relevant, then the published resource is incorporated in order to more fully describe the state of the art to which the present invention pertains, and/or to provide such teachings as are generally known to those skilled in the art, as may be applicable. However, it is specifically stated that a citation of a published resource herein shall not be construed as an admission that such is prior art to the present invention. Also, in the event that one or more of the incorporated published resources differs from or contradicts this application, including but not limited to defined terms, term usage, described techniques, or the like, this application controls.

While various embodiments of the present invention have been shown and described herein, it is emphasized that such embodiments are provided by way of example only. Numerous variations, changes and substitutions may be made without departing from the invention herein in its various embodiments. Specifically, and for whatever reason, for any grouping of compounds, nucleic acid sequences, polypeptides including specific proteins including functional enzymes, metabolic pathway enzymes or intermediates, elements, or other compositions, or concentrations stated or otherwise presented herein in a list, table, or other grouping (such as metabolic pathway enzymes shown in a figure), unless clearly stated otherwise, it is intended that each such grouping provides the basis for and serves to identify various subset embodiments, the subset embodiments in their broadest scope comprising every subset of such grouping by exclusion of one or more members (or subsets) of the respective stated grouping. Moreover, when any range is described herein, unless clearly stated otherwise, that range includes all values therein and all sub-ranges therein. Accordingly, it is intended that the invention be limited only by the spirit and scope of appended claims, and of later claims, and of either such claims as they may be amended during prosecution of this or a later application claiming priority hereto.

### EXAMPLES SECTION

Examples 1 to 3 are directed to reduction of conversion of 3-HP to its aldehydes, examples 4 to 7 demonstrate non-limiting approaches to providing genetic modifications for 3-HP production, and Example 8 discloses a combination of these features, and the remaining general prophetic examples provide guidance on how the invention may be utilized in a range of microorganism species. Other general prophetic examples follow regarding practice of embodiments of the invention in additional microorganism species.

Where there is a method in the following examples to achieve a certain result that is commonly practiced in two or more specific examples (or for other reasons), that method may be provided in a separate Common Methods section that follows the examples. Each such common method is incorporated by reference into the respective specific example that so refers to it. Also, where supplier information is not complete in a particular example, additional manufacturer information may be found in a separate Summary of Suppliers section that may also include product code, catalog number, or other information. This information is intended to be incorporated in respective specific examples that refer to such supplier and/or product.

In the following examples, efforts have been made to ensure accuracy with respect to numbers used (e.g., amounts, temperatures, etc.), but some experimental error and deviation should be accounted for. Unless indicated otherwise, temperature is in degrees Celsius and pressure is at or near atmospheric pressure at approximately 5340 feet (1628 meters) above sea level. It is noted that work done at external analytical and synthetic facilities was not conducted at or near atmospheric pressure at approximately 5340 feet (1628 meters) above sea level. All reagents, unless otherwise indicated, were obtained commercially. Species and other phylogenic identifications provided in the examples and the Common Methods Section are according to the classification known to a person skilled in the art of microbiology.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “g” means gram(s), “μg” or “ug” means microgram(s) and “ng” means nanogram(s), “PCR” means polymerase chain reaction, “OD” means optical density, “OD₆₀₀” means the optical density measured at a wavelength of 600 nm, “kDa” means kilodaltons, “g” means the gravitation constant, “bp” means base pair(s), “kbp” means kilobase pair(s), “% w/v” means weight/volume percent, % v/v” means volume/volume percent, “IPTG” means isopropyl-μ-D-thiogalactopyranoiside, “RBS” means ribosome binding site, “rpm” means revolutions per minute, “HPLC” means high performance liquid chromatography, and “GC” means gas chromatography. As disclosed above, “3-HP” means 3-hydroxypropionic acid, “3-HPA” means 3-hydroxypropionaldehyde, and

“MSA” means malonate semialdehyde. Also, 10̂5 and the like are taken to mean 10⁵ and the like.

### Example 1

### E. coli Mutants with Decreased Conversion of 3-HP to an Aldehyde

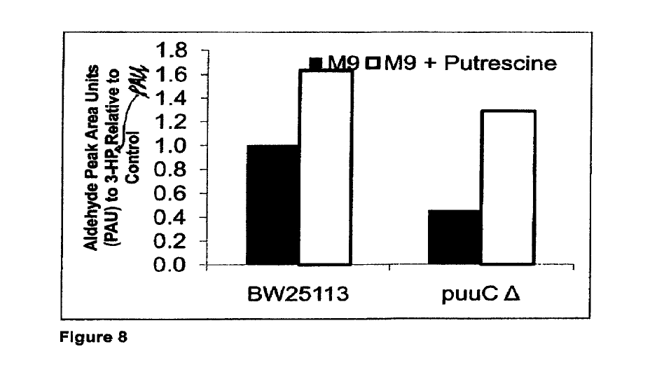

The control E. coli strain BW25113 and 22 of its derivatives, each derivative having a deletion of a respective one of 22 aldehyde dehydrogenases or related genes (predicted aldehyde dehydrogenases via homology, www.ecocyc.org) were cultured as described in methods in the Common Methods Section. Strains were obtained from the Keio collection that had deletions of the aldehyde dehydrogenase genes listed in Table 1, which provides sequence listing numbers of 22 genes (SEQ ID NOs. 1-22) and the amino acid sequences encoded by these genes (SEQ ID NOs. 23-44). The Keio collection was obtained from Open Biosystems (Huntsville, Ala. USA 35806). These strains each contain a kanamycin marker in place of the deleted gene. For more information concerning the Keio Collection and the curing of the kanamycin cassette please refer to: Baba, T et al (2006). Construction of Escherichia coli K12 in-frame, single-gene knockout mutants: the Keio collection. Molecular Systems Biology doi:10.1038/msb4100050 and Datsenko K A and B L Wanner (2000). One-step inactivation of chromosomal genes in Escherichia coli K-12 using PCR products. PNAS 97, 6640-6645. Data is shown in FIG. 6 showing the effect of each of these gene deletions on the ratio of intracellular aldehyde to 3-HP, when exposed to an extracellular source of 3-HP. This data confirms the production of an aldehyde in response to 3-HP in E. coli. Deletions of 20 of these genes are shown to decrease levels of this aldehyde in response to 3-HP in E. coli. Genes with significant decrease in such conversion include puuC (aldH), proA, ygbJ, yneI, eutE and betB.

Of particular importance is puuC which has previously been identified to convert 3-HP to 3-HPA and has been called aldH. This gene is involved in putrescine metabolism and known to be induced by putrescine. Thus, increased putrescine levels which are needed for 3-HP tolerance can induce the production on the puuC gene product and conversion of 3-HP to 3-HPA. A greater level of this aldehyde in response to 3-HP in elevated levels of putrescine is shown in FIG. 7. However, the effect of putrescine is not limited to an effect of the puuC gene product alone. As FIG. 8 shows, elevated levels of this aldehyde in response to 3-HP are induced by putrescine even in a strain lacking the puuC gene.

Based on these results, deletions of these 20 genes or combinations of deletions of these 20 genes can be used to decrease the levels of this aldehyde in response to the presence of3-HP and can conceivably increase tolerance to 3-HP. Table 1 provides a listing of these genes and includes the names of their enzyme products and sequence identification numbers both for the nucleic acid sequences and the encoded enzymes. Such genetic modifications may be combined with other genetic modifications described and/or exemplified herein.

### Example 2

### Preparation and Evaluation Over-Expressed Dehydrogenases

Aldehyde dehydrogenase genes were amplified by PCR from genomic E. coli DNA using the primers in Table 3 (SEQ ID NOs. 045 to 118) for the respective genes of Table 1. Open reading frames (ORFs) were amplified from the start codon to the amino acid preceding the stop codon to allow for expression of the hexa-histidine tag encoded by the vector. PCR products were isolated by gel electrophoresis and gel purified using Qiagen gel extraction (Valencia, Calif. USA, Cat. No. 28706) following the manufacturer's instructions. Gel purified dehydrogenase gene open reading frames (see Table 1 for SEQ ID NOs) were then cloned into pTrcHis2-Topo vector (SEQ ID NO:119), Invitrogen Corp, Carlsbad, Calif., USA) following manufacturer's instructions. DNA was transformed and cultured. Subsequently, DNA from colonies was miniprepped and screened by restriction digestion. All isolated plasmids were sequenced verified by the DNA sequencing services of Genewiz Corporation (S. Plainfield, N.J. USA). Of the genes listed in Table 1, the following were cloned according to this procedure: aldA; aldB; betB; eutG; fucO; gidA; gnd; ldhA; proA; puuC; sad; and ssuD (respective nucleic acid and amino acid sequence numbers provided in Table 1, incorporated into this Example). Protein expression was confirmed by Western Blot analysis described below for the following of these cloned genes: aldA; aldB; betB; eutG; fucO; gldA; gnd; ldhA; puuC; and ssuD.

Confirmation of Protein Expression by Western Blot

Bacterial cultures were grown in LB+Amp 200 ug/mL to an approximate O.D. of 0.6-0.7 at 37 degrees Celsius. Protein expression was induced with 1 mM final concentration IPTG and cultures were further grown overnight. For each culture, 1 mL aliquots of bacterial culture were taken immediately before induction and prior to harvesting at 24 hr. Whole cell extracts were prepared for Western Blot analysis. Samples were pelleted by centrifugation and resuspended in 100 uL of SDS sample buffer (Tris-Cl pH 6.8, SDS, glycerol, β-mercaptoethanol, Bromophenol blue), boiled for 5 minutes and spun at 17,000 G for 5 minutes. Samples prepared from un-induced and induced cultures (10 microliters) were loaded on a 10% pre-cast SDS-PAGE gel (BioRad Ready Gel Tris-HCl Gel-161-1101) electrophoresis was carried out using a BioRad Mini-Protean II system according to manufacturer's instructions. SDS gels were transferred to nitrocellulose membrane using the same BioRad Mini-Protean II wet transfer system according to manufacturer's specifications.

Membranes were blocked for 1 hour at room temperature using PBST (NaCl, KCl, Na₂HPO₄, KH₂PO₄, Tween 20)+5% w/v nonfat dry milk. Blots were then probed with a rabbit polyclonal anti-6× HIS-HRP antibody (AbCam Ab1187, 1:5000 dilution) in PBST+5% w/v nonfat dry milk for 1 hour at room temperature, washed 4 times in PBST for 5 minutes, and followed by developing with TMB substrate (Promega TMB Stabilized Substrate for HRP, cat#W4121). Protein expression was assessed by the presence or absence of bands at the expected molecular weight for each proteins of interest. Samples showing positive protein expression were subjected to protein purification as described below.

Whole-Cell Protein Extraction

Whole cell lysate and purified protein samples for these dehydrogenase genes were prepared as follow: 30mL bacterial cultures were grown in LB+Amp 200 ug/mL to an approximate O.D. of 0.6-0.7. Protein expression was induced with 1 mM final concentration IPTG and grown overnight. Cells were pelleted at 3220 G for 10 minutes. Pellets were resuspended in 1 mL lysis buffer (25 mM Tris pH 8, 500 mM NaCl, 1.5 mg/mL lysozyme, and Complete Protease Inhibitor Cocktail Roche (Basel, Switzerland) and incubated on ice for 15 minutes. Resuspensions were sonicated briefly (3 time 30 s pulses). Lysates were then cleared by centrifugation at 10,000 G. Clearer lysates were kept for further purification as well as used in enzyme assays as described below. All steps were performed at 4 degrees Celsius unless otherwise stated.

Protein Purification

For protein purifications, portions of the cleared lysates were loaded onto Ni-NTA spin columns (Qiagen, Valencia Calif. USA). After binding his-tagged protein, columns were washed three times with high-salt wash buffer (25 mM Tris pH 8, 500 mM NaCl, 1 mM imidazol). Columns were then washed once with a low-salt wash buffer (25 mM Tris pH 8, 100 mM NaCl, 1 mM imidazol). Purified protein was eluted in 200 uL elution buffer (25 mM Tris pH 8, 100 mM NaCl, 300 mM imidazol). Purification of each protein was evaluated by SDS-PAGE gel analysis to assess yield and purity

Enzyme Activity Assays for Dehydrogenase Enzymes with 3-HP as a Substrate

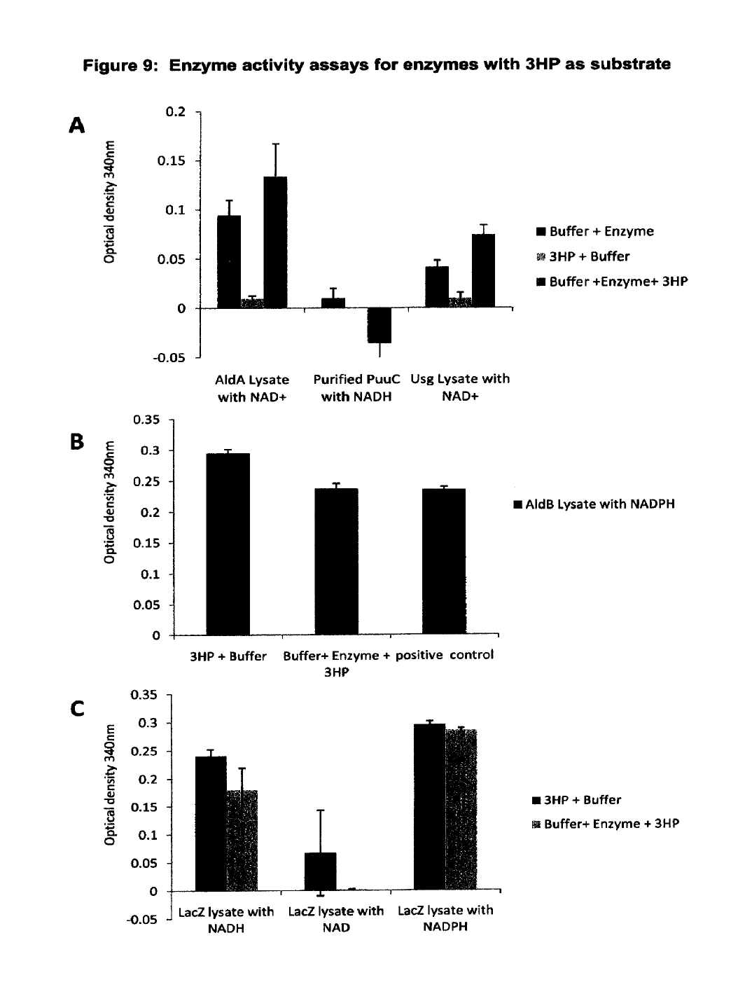

Several dehydrogenases showed enzymatic activity using 3-HP as a substrate. Samples of these enzymes were isolated either as clarified lysates or as purified enzymes as described in the method reported above. As these dehydrogenases use NAD+, NADH, NADP+, NADPH or all of these molecules as cofactors for their reactions depending on reaction direction, all enzymes where tested with their known cofactors. For enzymes where the specific cofactors have not been determined or maybe unclear, all possible cofactors were evaluated. Of the cloned and over-expressed genes, aldA, aldB, puuC, and usg (SEQ ID NO:120 for nucleic acid sequence, SEQ ID NO: 121 for encoded enzyme, which is an E. coli aldehyde dehydrogenase not listed in Table 1) showed activity in our assays. The results of these assays are shown in FIGS. 9A-C.

A spectrophotometric assay was used to evaluate enzyme activity. As the reduced forms of these cofactors (NADH and NADPH) possess a strong absorption peaks at 340 nm, the ability of these dehydrogenases to react with 3-HP as a substrate could be monitored by comparing the increase in absorption at 340 nm for reactions reducing NAD+ or NADP+, or by decrease in absorption at 340 nm for reactions oxidizing NADH or NADPH. Replicates of reactions were carried out to compare reactions in the presence or absence or 3-HP, and with and without enzyme. Enzymatic activities were confirmed by comparing the change in the 340 nm absorption values after 1 hour incubations to reactions performed in buffer containing 1 mM cofactor as a baseline. Comparisons between buffer with 3-HP, buffer with enzyme, and buffer with 3-HP and enzyme are shown in FIGS. 9A and 9B. As further controls, over-expressed LacZ lysate was assess for its ability to oxidize or reduce cofactors in the presence of 3-HP. None of this LacZ control lysate showed no activity as shown in FIG. 9C. Furthermore, activity of the purified aldB enzyme was confirmed with its natural substrate (1 mM acetate) as in FIG. 9B.

Reactions were carried out using one of two reaction buffers. AldA, AldB, LacZ, and Usg reactions were performed in a buffer consisting of 100 mM potassium phosphate buffer pH 7.4 with 50 mM sodium chloride. Likewise, puuC reactions were performed in a buffer consisting of 200 mM sodium bicarbonate pH 9.2 with 10 mM dithiothreitol and 30 micromolar ferrous sulphate. Where stated, all cofactors were used at 1 mM in the final reaction buffer. In addition, 3-HP was also used at 1 mM in the final reaction buffer. After one hour incubations at room temperature, the samples were diluted 1 to 20 in water and measured with a Beckmann DU530 spectrometer set at 340 nm. These results show the aldA, aldB, puuC, and usg showed activity in the presence of 3-HP and cofactor.

### Example 3

### Preparation and Evaluation of E. coli Modified to Disrupt Aldehyde Dehydrogenase Genes and Having 3-HP Production Genetic Modification

Construction of pSC-B-Ptpia:mcr

The protein sequence (SEQ ID NO:122) of the malonyl-coA reductase gene (mcr) from Chloroflexus aurantiacus was codon optimized for E. coli according to a service from DNA 2.0 (Menlo Park, Calif. USA), a commercial DNA gene synthesis provider. This synthetic codon-optimized nucleic acid sequence was synthesized with an EcoRI restriction site before the start codon and also comprised a HindIII restriction site following the termination codon. In addition a Shine Delgarno sequence (i.e., a ribosomal binding site) was placed in front of the start codon preceded by the EcoRI restriction site. This gene construct was synthesized by DNA 2.0 and provided in a pJ206 vector backbone. This plasmid, comprising this codon-optimized nucleic acid sequence for mcr, was designated pJ206:mcr (SEQ ID NO:123). This synthesized plasmid was used as a template to amplify the mcr gene in order to construct a version of mcr under the control of a constitutive promoter derived from the rpiA gene from E. coli.

To create plasmids containing the mcr gene under the control of a constitutive rpiA promoter, both the codon optimized mcr gene and a tpiA promoter were amplified via a polymerase chain reaction. For the mcr gene, the polymerase chain reaction was performed with the forward primer being TCGTACCAACCATGGCCGGTACGGGTCGTTTGGCTGGTAAAATTG (SEQ ID NO:124) containing a NcoI site that incorporates the start methionine for the protein sequence, and the reverse primer being /5′PHOS/GGATTAGACGGTAATCGCACGACCG (SEQ ID NO:125) using the synthesized pJ206:mcr plasmid described above as template. For the tpiA promoter, the polymerase chain reaction was performed with the forward primer being GGGAACGGCGGGGAAAAACAAACGTT (SEQ ID NO:126), and the reverse primer being GGTCCATGGTAATTCTCCACGCTTATAAGC (SEQ ID NO:127) containing an NcoI site as template using genomic DNA isolated from a K12 strain as template. Both polymerase chain reaction products were purified using a PCR purification kit from Qiagen Corporation (Valencia, Calif., USA) using the manufactures instructions. Following purification, the mcr products and the tpiA promoter products were subjected to enzymatic restriction digestion with the enzyme NcoI. Restriction enzymes were obtained from New England BioLabs (Ipswich, Mass. USA), and used according to manufacturer's instructions. The digestion mixtures were separated by agarose gel electrophoresis, and visualized under UV transillumination as described under Methods. Agarose gel slices containing the DNA piece corresponding to the amplified mcr gene product and the tpiA promoter product were cut from the gel and the DNA recovered with a standard gel extraction protocol and components from Qiagen according to manufacturer's instructions. The recovered products were ligated together with T4 DNA ligase obtained from New England BioLabs (Ipswich, Mass. USA) according to manufacturer's instructions.

Since the ligation reaction can result in several different products, the desired product corresponding to the tpiA promoter ligated to the mcr gene was amplified by polymerase chain reaction and isolated by a second gel purification. For this polymerase chain reaction, the forward primer was GGGAACGGCGGGGAAAAACAAACGTT (SEQ ID NO:128), and the reverse primer was /5′PHOS/GGATTAGACGGTAATCGCACGACCG (SEQ ID NO: 125), and the ligation mixture was used as template. The digestion mixtures were separated by agarose gel electrophoresis, and visualized under UV transillumination as described under Methods. Agarose gel slices containing the DNA piece corresponding to the amplified promoter-gene fusion was cut from the gel and the DNA recovered with a standard gel extraction protocol and components from Qiagen according to manufacturer's instructions. This extracted DNA was inserted into a pSC-B vector using the Blunt PCR Cloning kit obtained from Stratagene Corporation (La Jolla, Calif., USA) using the manufactures instructions. Colonies were screened by colony polymerase chain reactions. Plasmid DNA from colonies showing inserts of correct size were cultured and miniprepped using a standard miniprep protocol and components from Qiagen according to the manufactures instruction. Isolated plasmids were checked by restrictions digests and confirmed by sequencing. The sequenced-verified isolated plasmids produced with this procedure were designated pSC-B-PtpiA:mcr (SEQ ID NO:129).

Construction of pBT-3-Ptpia:mcr

The insertion region pSC-B-PtpiA:mcr plasmid containing mcr gene under the control of a constitutive tpiA promoter was transferred to a pBT-3 vector. The pBT-3 vector (SEQ ID NO:130) provides for a broad host range origin or replication and a chloramphenicol selection marker.

For transferring the promoter-gene fusion into the pBT-3 vector, a pBT-3 vector was produced by polymerase chain amplification. For this polymerase chain reaction, the forward primer was AACGAATTCAAGCTTGATATC (SEQ ID NO:131), and the reverse primer was GAATTCGTTGACGAATTCTCT (SEQ ID NO:132), using pBT-3 as template. The amplified product was subjected to treatment with DpnI to restrict the methylated template DNA, and the mixture was separated by agarose gel electrophoresis, and visualized under UV transillumination as described under Methods. Agarose gel slices containing the DNA piece corresponding to amplified pBT-3 vector product was cut from the gel and the DNA recovered with a standard gel extraction protocol and components from Qiagen according to manufacturer's instructions.

For transferring the insertion region pSC-B-PtpiA:mcr plasmid containing mcr gene under the control of a constitutive tpiA promoter, the insertion region was produced by polymerase chain reaction. For this polymerase chain reaction, the forward primer was /5phos//5phos/GGAAACAGCTATGACCATGATTAC (SEQ ID NO:133), and the reverse primer was /5phos/TTGTAAAACGACGGCCAGTGAGCGCG (SEQ ID NO:134), using pSC-B-PtpiA:mcr as template. The amplified promoter-gene fusion insert was separated by agarose gel electrophoresis, and visualized under UV transillumination as described under Methods. Agarose gel slices containing the DNA piece corresponding to the amplified promoter-gene fusion was cut from the gel and the DNA recovered with a standard gel extraction protocol and components from Qiagen according to manufacturer's instructions. This insert DNA was ligated into the prepared pBT-3 vector prepared as described above with T4 DNA ligase obtained from New England Biolabs (Bedford, Mass., USA), following the manufactures instructions. Ligation mixtures were transformed into E. coli 10 G cells obtained from Lucigen Corp according to the manufactures instructions. Colonies were screened by colony polymerase chain reactions. Plasmid DNA from colonies showing inserts of correct size were cultured and miniprepped using a standard miniprep protocol and components from Qiagen according to the manufactures instruction. Isolated plasmids were checked by restrictions digests and confirmed by sequencing. The sequenced-verified isolated plasmids produced with this procedure were designated pBT-3-PtpiA:mcr (SEQ ID NO:135).

Construction of E. coli Strains with Multiple Aldehyde Dehydrogenase Gene Deletions

Strain Construction:

E. coli strain JW1375 was obtained from the Yale E. coli genetic stock center (E. coli Genetic Stock Center, New Haven, Conn. 06520-8103, http://cgsc.biology.yale.edu/index.php). The genotype of this strain is F-, Δ(araD-araB)567, ΔlacZ4787(::rrnB-3), LAM-, rph-1, Δ(rhaD-rhaB)568, hsdR514, ΔldhA744::kan. The strain was transformed by routine methods with the plasmid pCP20, which was also obtained from the Yale E. coli Genetic Stock Center. The strain was transformed with the pCP20 plasmids and the kanamycin resistance cured per the method below. The resulting strain BX_{—}00013.0 had the following genotype: F-, Δ(araD-araB)567, ΔlacZ4787(::rrnB-3), LAM-, rph-1, (rhaD-rhaB)568, hsdR514, ΔldhA:frt. This genotype was confirmed by PCR amplification of the region surrounding the ldhA gene, per the screening protocol given below with primers homologous to sequences farther upstream or downstream of the original PCR product.

Subsequent additional genetic modifications in the BX_{—}00013.0 background were constructed in 2 ways. In both methods PCR fragments containing the kanamycin marker gene replacement of any gene along with 300 base pairs of upstream and downstream homology was amplified by polymerase chain reaction from E. coli single gene deletion clones obtained from the Yale Genetic stock center. In the case of constructing strains with ΔldhA:frt, ΔpflB:frt and ΔldhA:frt, ΔpflB:frt, ΔfruR:frt genotypes, these fragments were electroporated into electrocompetent cells and colonies selected on Luria Broth agar plates containing 20 micrograms/nil kanamycin at 37 degrees Celsius. Strains were screened by the protocol given below. Between each genetic deletion, kanamycin cassettes were cured with pCP20 plasmid as described below. Subsequent combinations of genetic deletions were constructed using the respective PCR fragments into electrocompetent cell lines expressing plasmid born phage based recombination machinery per the standard recombineering methodologies and reagents supplied by Gene Bridges (Gene Bridges GmbH, Dresden, Germany, www.genebridges.com). Again strains were screened and cured by the protocols below. Table 4 gives a list of constructed strains comprising the indicated combination of deleted genes.

The strains listed in Table 4 were also subsequently transformed with the plasmid pBT-3-ptpiA-mcr (SEQ ID 135) which expresses the mcr (malonyl-coA reductase) gene which can convert malonyl-coA into 3-HP, conferring in these strains the ability to produce 3-HP.

Amplification of Kanamycin Cassettes for Homologous Gene Replacement

E. coli strains were obtained from the Yale E. coli genetic stock center. These strains have a kanamycin resistance marker replacing the respective genes. This marker along with 300 base pairs of upstream and downstream homology was amplified by polymerase chain reaction: in 14 μL of sterile water, 0.5 μL of upstream primer, 0.5 μL of internal kanamycin primer K1, and 15 μL of EconTaq®PLUS GREEN 2× Master Mix (Lucigen, 30033-2). PCR was performed using a Stratagene Robocycler thermocycler (Stratagene, Cedar Creek, Tex. USA) with the following settings: 94° C. for 10 minutes, then 32 cycles of 94° C. for 1 minute, 52° C. for 1 minute, and 72° C. for 2 minutes 30 seconds, with a final extension at 72° C. for 10 minutes. The PCR reaction was checked by running 10 μL of each reaction on an agarose gel. PCR fragments were used to transform electrocompetent cells. Primers used in the amplification of these markers from the appropriate strains are given in Table 5 (SEQ ID NOs: 136 to 145).

Curing of Kanamycin Cassettes and pCP20 Plasmid

Colonies containing the pCP20 were isolated on Luria Broth agar plates containing 20 micrograms/ml chloramphenicol at 30 degrees Celsius and subsequently grown at 42 degrees Celsius, which simultaneously cured or removed the plasmid and induced the plasmid borne flp recombinase which removed the kanamycin resistance cassette from the genome leaving an frt site.

Subsequently the pflB and fruR genes were deleted sequentially in the BX_{—}00013.0 background. This was done as follows: E. coli strains JW0866 and JW0078 were obtained from the Yale E. coli genetic stock center. These strains have a kanamycin resistance marker replacing the pflB and fruR genes respectively. This marker along with 300 base pairs of upstream and downstream homology was amplified by polymerase chain reaction as follows: in 14 μL of sterile water, 0.5 μL of upstream primer, 0.5 μL of internal kanamycin primer K1, and 15 μL of EconTaq®PLUS GREEN 2× Master Mix (Lucigen, 30033-2). PCR was performed using a Stratagene Robocycler thermocycler (Stratagene, Cedar Creek, Tex. USA) with the following settings: 94° C. for 10 minutes, then 32 cycles of 94° C. for 1 minute, 52° C. for 1 minute, and 72° C. for 2 minutes 30 seconds, with a final extension at 72° C. for 10 minutes. The PCR reaction was checked by running 10 μL of each reaction on an agarose gel. PCR fragments were used to transform electrocompetent cells.

Screening Protocol:

The following PCR protocol was designed to screen and confirm single and multiple aldehyde dehydrogenase deletions in E. coli. The primers used in these methods, and their respective sequence numbers (SEQ ID NOs:146 to 158) are provided in Table 6.

A PCR test was designed to screen the appropriate number of colonies (up to greater than 100, based on the method of introduction of gene deletion(s)), compared to a positive deletion control for a desired genetic modification. Strain screening was performed by setting up reaction mixtures containing a single colony suspension in 14 μL of sterile water, 0.5 μL of upstream primer, 0.5 μL of internal kanamycin primer K1 (See Wanner, Barry L., and Kirin A. Datsenko. One-step inactivation of chromosomal genes in Escherichia coli K-12 using PCR products. Proc. Natl. Acad. Sci. USA, 97(12), 6640-6645), and 15 μL of EconTaq®PLUS GREEN 2× Master Mix (Lucigen, 30033-2). PCR was performed using a Stratagene Robocycler thermocycler (Stratagene, Cedar Creek, Tex. USA) with the following settings: 94° C. for 10 minutes, then 32 cycles of 94° C. for 1 minute, 52° C. for 1 minute, and 72° C. for 2 minutes 30 seconds, with a final extension at 72° C. for 10 minutes. The PCR reaction was checked by running 10 μL of each reaction on an agarose gel. Positive clones were re-streaked onto the appropriate selective media plate.

A second PCR test was designed to determine if cumulative background modifications were maintained during subsequent rounds of strain construction. Strain confirmation was performed for each genetic modification made to that point compared to the background strain. A series of reaction mixtures was set up for positive clones containing a colony suspension in 14 μL it of sterile water, 1 μL of primer mix, and 15 μL of EconTaq®PLUS GREEN 2× Master Mix (Lucigen). The primer mix contained either 0.5 μL each of upstream and downstream homology primers for background ALD deletions or 0.5 μL of upstream homology primer and 0.5 μL of internal kanamycin primer K1 for the additional modification. PCR was performed using a Stratagene Robocycler thermocycler (Stratagene, Cedar Creek, Tex. USA) with the following settings: 94° C. for 10 minutes, then 32 cycles of 94° C. for 1 minute, 52° C. for 1 minute, and 72° C. for 2 minutes 30 seconds, with a final extension at 72° C. for 10 minutes. The PCR reaction was checked by running 10 μL of each reaction on an agarose gel. Final strains were documented and made into freezer stocks for long-term storage.

### Example 4

### Genetic Modification/Introduction of Malonyl-CoA Reductase for 3-HP Production in E. coli DF40

The nucleotide sequence for the malonyl-coA reductase gene (“mcr” or “MCR”) from Chloroflexus aurantiacus was codon optimized for E. coli according to a service from DNA 2.0 (Menlo Park, Calif. USA), a commercial DNA gene synthesis provider. This codon-optimized gene sequence incorporated an EcoRI restriction site before the start codon and was followed by a HindIII restriction site. In addition a Shine Delgarno sequence (i.e., a ribosomal binding site) was placed in front of the start codon preceded by an EcoRI restriction site. This gene construct was synthesized by DNA 2.0 and provided in a pJ206 vector backbone. Plasmid DNA pJ206 containing the synthesized mcr gene was subjected to enzymatic restriction digestion with the enzymes EcoRI and HindIII obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the mcr gene was cut from the gel and the DNA recovered with a standard gel extraction protocol and components from Qiagen (Valencia, Calif. USA) according to manufacturer's instructions. An E. coli cloning strain bearing pKK223-aroH was obtained as a kind a gift from the laboratory of Prof. Ryan T. Gill from the University of Colorado at Boulder. Cultures of this strain bearing the plasmid were grown by standard methodologies and plasmid DNA was prepared by a commercial miniprep column from Qiagen (Valencia, Calif. USA) according to manufacturer's instructions. Plasmid DNA was digested with the restriction endonucleases EcoRI and HindIII obtained from New England Biolabs (Ipswich, Mass. USA) according to manufacturer's instructions. This digestion served to separate the aroH reading frame from the pKK223 backbone. The digestion mixture was separated by agarose gel electrophoresis, and visualized under UV transillumination as described in Subsection II of the Common Methods Section. An agarose gel slice containing a DNA piece corresponding to the backbone of the pKK223 plasmid was cut from the gel and the DNA recovered with a standard gel extraction protocol and components from Qiagen according to manufacturer's instructions.

Pieces of purified DNA corresponding to the mcr gene and pK223 vector backbone were ligated and the ligation product was transformed and electroporated according to manufacturer's instructions. The sequence of the resulting vector termed pKK223-mcr (SEQ ID NO:159) was confirmed by routine sequencing performed by the commercial service provided by Macrogen(USA). pKK223-mcr confers resistance to beta-lactamase and contains the mcr gene of C. aurantiacus under control of a ptac promoter inducible in E. coli hosts by IPTG. The expression clone pKK223-mcr and pKK223 control were transformed into both E. coli K12 and E. coli DF40 (E. Coli Genetic Stock Center, Yale Univ., New Haven, Conn. USA) via standard methodologies. (Sambrook and Russell, 2001).

3-HP production of E. coli DF40+pKK223-MCR was demonstrated at 10 mL scale in M9 minimal media. Cultures of E. coli DF40, E. coli DF40+pKK223, and E. coli DF40+pKK223-MCR were started from freezer stocks by standard practice (Sambrook and Russell, 2001) into 10 mL of LB media plus 100 ug/mL ampicillin where indicated and grown to stationary phase overnight at 37 degrees shaking at 225 rpm overnight. In the morning, these cells from these cultures were pelleted by centrifugation and resuspended in 10 mL of M9 minimal media plus 5%(w/v) glucose. This suspension was used to inoculate 5% (v/v) fresh 10 ml cultures [5% (v/v)] in M9 minimal media plus 5%(w/v) glucose plus 100 ug/mL ampicillin where indicated. These cultures were grown in at least triplicate, with 1 mM IPTG added. To monitor growth of these cultures, Optical density measurements (absorbance at 600 nm, 1 cm pathlength), which correlate to cell numbers, were taken at time=0 and every 2 hrs after inoculation for a total of 12 hours. After 12 hours, cells were pelleted by centrifugation and the supernatant collected for analysis of 3-HP production as described under “Analysis of cultures for 3-HP production” in the Common Methods section.

Results

3-HP was determined present by HPLC analysis.

### Example 5

### One-Liter Scale Bio-Production of 3-HP Using E. coli DF40+pKK223+MCR

Using E. coli strain DF40+pKK223+MCR that was produced in accordance with Example 4 above, a batch culture of approximately 1 liter working volume was conducted to assess microbial bio-production of 3-HP. E. coli DF40+pKK223+MCR was inoculated from freezer stocks by standard practice (Sambrook and Russell, 2001) into a 50 mL baffled flask of LB media plus 200 μg/mL ampicillin where indicated and grown to stationary phase overnight at 37° C. with shaking at 225 rpm. In the morning, this culture was used to inoculate (5% v/v) a 1-L bioreactor vessel comprising M9 minimal media plus 5% (w/v) glucose plus 200 μg/mL ampicillin, plus 1 mM IPTG, where indicated. The bioreactor vessel was maintained at pH 6.75 by addition of 10 M NaOH or 1 M HCl, as appropriate. The dissolved oxygen content of the bioreactor vessel was maintained at 80% of saturation by continuous sparging of air at a rate of 5 L/min and by continuous adjustment of the agitation rate of the bioreactor vessel between 100 and 1000 rpm. These bio-production evaluations were conducted in at least triplicate. To monitor growth of these cultures, optical density measurements (absorbance at 600 nm, 1 cm path length), which correlates to cell number, were taken at the time of inoculation and every 2 hrs after inoculation for the first 12 hours. On day 2 of the bio-production event, samples for optical density and other measurements were collected every 3 hours. For each sample collected, cells were pelleted by centrifugation and the supernatant was collected for analysis of 3-HP production as described per “Analysis of cultures for 3-HP production” in the Common Methods section, below. Preliminary final titer of 3-HP in this 1-liter bio-production volume was calculated based on HPLC analysis to be 0.7 g/L 3-HP. It is acknowledged that there is likely co-production of malonate semialdehyde, or possibly another aldehyde, or possibly degradation products of malonate semialdehyde or other aldehydes, that are indistinguishable from 3-HP by this HPLC analysis.

### Example 6

### Genetic Modification/Introduction of Malonyl-CoA Reductase for 3-HP Production in Bacillus subtilis

For creation of a 3-HP production pathway in Bacillus Subtilis the codon optimized nucleotide sequence for the malonyl-coA reductase gene from Chloroflexus aurantiacus that was constructed by the gene synthesis service from DNA 2.0 (Menlo Park, Calif. USA), a commercial DNA gene synthesis provider, was added to a Bacillus Subtilis shuttle vector. This shuttle vector, pHT08 (SEQ ID NO:160), was obtained from Boca Scientific (Boca Raton, Fla. USA) and carries an inducible Pgrac IPTG-inducible promoter.

This mcr gene sequence was prepared for insertion into the pHT08 shuttle vector by polymerase chain reaction amplification with primer 1 (5′GGAAGGATCCATGTCCGGTACGGGTCG-3’) (SEQ ID NO:161), which contains homology to the start site of the mcr gene and a BamHI restriction site, and primer 2 (5′-Phos-GGGATTAGACGGTAATCGCACGACCG-3′) (SEQ ID NO:162), which contains the stop codon of the mcr gene and a phosphorylated 5′ terminus for blunt ligation cloning. The polymerase chain reaction product was purified using a PCR purification kit obtained from Qiagen Corporation (Valencia, Calif. USA) according to manufacturer's instructions. Next, the purified product was digested with BamHI obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to the mcr gene was cut from the gel and the DNA recovered with a standard gel extraction protocol and components from Qiagen (Valencia, Calif. USA) according to manufacturer's instructions.

This pHT08 shuttle vector DNA was isolated using a standard miniprep DNA purification kit from Qiagen (Valencia, Calif. USA) according to manufacturer's instructions. The resulting DNA was restriction digested with BamHI and SmaI obtained from New England BioLabs (Ipswich, Mass. USA) according to manufacturer's instructions. The digestion mixture was separated by agarose gel electrophoresis, and visualized under UV transillumination as described in Subsection II of the Common Methods Section. An agarose gel slice containing a DNA piece corresponding to digested pHT08 backbone product was cut from the gel and the DNA recovered with a standard gel extraction protocol and components from Qiagen (Valencia, Calif. USA) according to manufacturer's instructions.

Both the digested and purified mcr and pHT08 products were ligated together using T4 ligase obtained from New England BioLabs (Ipswich, Mass. USA) according to manufacturer's instructions. The ligation mixture was then transformed into chemically competent 10 G E. coli cells obtained from Lucigen Corporation (Middleton Wis., USA) according to the manufacturer's instructions and plated LB plates augmented with ampicillin for selection. Several of the resulting colonies were cultured and their DNA was isolated using a standard miniprep DNA purification kit from Qiagen (Valencia, Calif. USA) according to manufacturer's instructions. The recovered DNA was checked by restriction digest followed by agarose gel electrophoresis. DNA samples showing the correct banding pattern were further verified by DNA sequencing. The sequence verified DNA was designated as pHT08-mcr, and was then transformed into chemically competent Bacillus subtilis cells using directions obtained from Boca Scientific (Boca Raton, Fla. USA). Bacillus subtilis cells carrying the pHT08-mcr plasmid were selected for on LB plates augmented with chloramphenicol.

Bacillus subtilis cells carrying the pHT08-mcr, were grown overnight in 5 ml of LB media supplemented with 20 ug/mL chloramphenicol, shaking at 225 rpm and incubated at 37 degrees Celsius. These cultures were used to inoculate 1% v/v, 75 mL of M9 minimal media supplemented with 1.47 g/L glutamate, 0.021 g/L tryptophan, 20 ug/mL chloramphenicol and 1 mM IPTG. These cultures were then grown for 18 hours in a 250 mL baffled Erlenmeyer flask at 25 rpm, incubated at 37 degrees Celsius. After 18 hours, cells were pelleted and supernatants subjected to GC/MS detection of 3-HP (described in Common Methods Section IIIb)). Trace amounts of 3-HP were detected with qualifier ions.

### Example 7

### Yeast Aerobic Pathway for 3HP Production (Prophetic)

The artificial chemically synthesized nucleic acid construct (SEQ ID NO:163), which is in a plasmid obtained from DNA2.0 (Menlo Park, Calif. USA), containing: 200 bp 5′ homology to ACC1,His3 gene for selection, Adh1 yeast promoter, BamHI and Spel sites for cloning of MCR, cyc 1 terminator, Tefl promoter from yeast and the first 200 bp of homology to the yeast ACC1 open reading frame will be constructed using gene synthesis (DNA 2.0, Menlo Park, Calif. USA). The MCR (malonyl Co-A reductase) open reading frame (SEQ ID NO:164), codon-optimized for E. coli from the natural C. aurantiacus sequence, will be cloned into the BamHI and Spel sites. This will allow for constitutive transcription by the adhl promoter. Following the cloning of MCR into the construct (SEQ ID NO:163) the genetic element (SEQ ID NO:165) will be isolated from the plasmid by restriction digestion and transformed into relevant yeast strains. The genetic element will knock out the native promoter of yeast ACC1 and replace it with MCR expressed from the adhl promoter and the Tefl promoter will now drive yeast ACC1 expression. The integration will be selected for by growth in the absence of histidine. Positive colonies will be confirmed by PCR. Expression of MCR and increased expression of ACC1 will be confirmed by RT-PCR.

An alternative approach that could be utilized to express MCR in yeast is expression of MCR from a plasmid. The genetic element containing MCR under the control of the ADH1 promoter could be cloned into a yeast vector such as pRS421 (SEQ ID NO:166) using standard molecular biology techniques creating a plasmid containing MCR (SEQ ID NO:167). A plasmid-based MCR could then be transformed into different yeast strains.

### Example 8

### Aldehyde Dehydrogenase Deletions Plus 3-HP Production in an E. coli Host Cell (Prophetic)

Deletions of the nucleic acid sequences encoding the aldA, aldB, and puuC genes are made in a selected E. coli strain, such as E. coli DF40 described above, using a RED/ET homologous recombination method, with kits supplied by Gene Bridges (Gene Bridges GmbH, Dresden, Germany, www.genebridges.com) according to manufacturer's instructions. The successful deletion of these genes, as confirmed by standard methodologies, such as PCR (see Example 2 above), or DNA sequencing, results in a suitable genetically modified microorganism for the following step.

The aforementioned genetically modified microorganism is transformed with a plasmid comprising malonyl-CoA-reductase gene (mcr) controlled by a constitutive or inducible promoter (see Example 4 for details of the plasmid's construction).

The genetically modified microorganism comprising the mcr addition and the deletions of aldA, aldB, and puuC (and optionally another aldehyde dehydrogenase, for example, usg, SEQ ID NO:120) is evaluated for production of 3-HP and its aldehydes. In a suitable media, such as those described herein, this microorganism produces less aldehydes, and more 3-HP, than either control microorganisms of the same selected strain that either lack mcr, or are supplied with mcr but lack the noted gene deletions.

In addition, at least one such embodiment results in a genetically modified microorganism that demonstrates, when in a culture system comprising a suitable media for growth and/or for production of 3-HP, increased productivity, yield, titer, and/or purity of 3-HP. Such increased parameters are assessed, as is common practice in the field, by comparison with a control lacking such genetic modifications.

It is noted that other gene deletion combinations, and other 3-HP production genes and enzymes (such as those of the 3-HP production pathways depicted in FIGS. 2, 3, 4A and 4B, also are prepared and evaluated.

Thus, based at least in part on the teachings herein, including the above examples various genetic modification combinations are identified, evaluated, and then are utilized to develop a genetically modified microorganism capable of reduced conversion of 3-HP to one of its aldehydes, and also, in various embodiments, in which 3-HP production genetic modifications also are provided. Genetic modifications include those directed to modify, such as disrupt, genes and enzymatic function of the enzymes they encode, that express or are aldehyde dehydrogenases that would otherwise convert 3-HP to one or more of its aldehydes.

In view of the above disclosure, the following pertain to exemplary methods of modifying specific species of host organisms that span a broad range of microorganisms of commercial value. These examples further support that the use of E. coli, although convenient for many reasons, is not meant to be limiting. As noted above, given the complete genome sequencing of a wide range of microorganisms and the high level of skill in the art, those skilled in the art are readily able to apply the teachings and guidance provided herein to other microorganisms of interest. The genetic modifications exemplified herein may be applied to numerous species by incorporating the same or analogous genetic modifications for a selected species. The following are non-limiting general prophetic examples directed to practicing embodiments of the present invention in other microorganism species.

### General Prophetic Example 9

Practice of Embodiments of the Invention in Rhodococcus erythropolis

A series of E. coli-Rhodococcus shuttle vectors are available for expression in R. erythropolis, including, but not limited to, pRhBR17 and pDA71 (Kostichka et al., Appl. Microbiol. Biotechnol. 62:61-68(2003)). Additionally, a series of promoters are available for heterologous gene expression in R. erythropolis (see for example Nakashima et al., Appl. Environ. Microbiol. 70:5557-5568 (2004), and Tao et al., Appl. Microbiol. Biotechnol. 2005, DOI 10.1007/s00253-005-0064). Targeted gene disruption of chromosomal genes in R. erythropolis may be created using the method described by Tao et al., supra, and Brans et al. (Appl. Environ. Microbiol. 66: 2029-2036 (2000)). These published resources are incorporated by reference for their respective indicated teachings and compositions.

The nucleic acid sequences required for providing an increase in 3-HP tolerance, as described above, optionally with nucleic acid sequences to provide and/or improve a 3-HP biosynthesis pathway, are cloned initially in pDA71 or pRhBR71 and transformed into E. coli. The vectors are then transformed into R. erythropolis by electroporation, as described by Kostichka et al., supra. The recombinants are grown in synthetic medium containing glucose and the bio-production of 3-HP may be followed using methods known in the art or described herein. Also, disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase.

### General Prophetic Example 10

Practice of Embodiments of the Invention in B. licheniformis

Most of the plasmids and shuttle vectors that replicate in B. subtilis are used to transform B. licheniformis by either protoplast transformation or electroporation. The nucleic acid sequences required for improvement of 3-HP tolerance, and/or for 3-HP biosynthesis are isolated from various sources, codon optimized as appropriate, and cloned in plasmids pBE20 or pBE60 derivatives (Nagarajan et al., Gene 114:121-126 (1992)). Methods to transform B. licheniformis are known in the art (for example see Fleming et al. Appl. Environ. Microbiol., 61(11):3775-3780 (1995)). These published resources are incorporated by reference for their respective indicated teachings and compositions.

The plasmids constructed for expression in B. subtilis are transformed into B. licheniformis to produce a recombinant microorganism that then demonstrates reduced conversion of 3-HP to it aldehydes, and, optionally, 3-HP bio-production. Disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase.

### General Prophetic Example 11

Practice of Embodiments of the Invention in Paenibacillus macerans

Plasmids are constructed as described above for expression in B. subtilis and used to transform Paenibacillus macerans by protoplast transformation to produce a recombinant microorganism that demonstrates reduced conversion of 3-HP to its aldehydes, and, optionally, 3-HP bio-production. Disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase.

### General Prophetic Example 12

Practice of Embodiments of the Invention in Alcaligenes (Ralstonia) Eutrophus (currently referred to as Cupriavidus necator).

Methods for gene expression and creation of mutations in Alcaligenes eutrophus are known in the art (see for example Taghavi et al., Appl. Environ. Microbiol., 60(10):3585-3591 (1994)). This published resource is incorporated by reference for its indicated teachings and compositions. Any of the nucleic acid sequences identified to improve 3-HP tolerance, and/or for 3-HP biosynthesis are isolated from various sources, codon optimized as appropriate, and cloned in any of the broad host range vectors described above, and electroporated to generate recombinant microorganisms that demonstrate improved 3-HP tolerance, and, optionally, 3-HP bio-production. The poly(hydroxybutyrate) pathway in Alcaligenes has been described in detail, a variety of genetic techniques to modify the Alcaligenes eutrophus genome is known, and those tools can be applied for engineering a genetically modified microorganism demonstrating reduced conversion of 3-HP to it aldehydes, and, optionally, a 3-HP-gena-toleragenic recombinant microorganism. Disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase.

### General Prophetic Example 13

### Practice of Embodiments of the Invention in Pseudomonas putida

Methods for gene expression in Pseudomonas putida are known in the art (see for example Ben-Bassat et al., U.S. Pat. No. 6,586,229, which is incorporated herein by reference for these teachings). Any of the nucleic acid sequences identified to improve 3-HP tolerance, and/or for 3-HP biosynthesis are isolated from various sources, codon optimized as appropriate, and cloned in any of the broad host range vectors described above, and electroporated to generate recombinant microorganisms that demonstrate improved 3-HP tolerance, and, optionally, 3-HP biosynthetic production. For example, these nucleic acid sequences are inserted into pUCP18 and this ligated DNA are electroporated into electrocompetent Pseudomonas putida KT2440 cells to generate recombinant P. putida microorganisms that exhibit reduced conversion of 3-HP to it aldehydes and, optionally, also comprise 3-HP biosynthesis pathways comprised at least in part of introduced nucleic acid sequences. Disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase.

### General Prophetic Example 14

Practice of Embodiments of the Invention in Lactobacillus plantarum

The Lactobacillus genus belongs to the Lactobacillales family and many plasmids and vectors used in the transformation of Bacillus subtilis and Streptococcus are used for lactobacillus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Several plasmids from Lactobacillus plantarum have also been reported (e.g., van Kranenburg R, Golic N, Bongers R, Leer R J, de Vos W M, Siezen R J, Kleerebezem M. Appl. Environ. Microbiol. 2005 March; 71(3): 1223-1230). Also, disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase. As noted for other species, genetic modification(s) directed to increase 3-HP production may also be provided in some embodiments.

### General Prophetic Example 15

Practice of Embodiments of the Invention in Enterococcus faecium, Enterococcus gallinarium, and Enterococcus faecalis

The Enterococcus genus belongs to the Lactobacillales family and many plasmids and vectors used in the transformation of Lactobacillus, Bacillus subtilis, and Streptococcus are used for Enterococcus. Non-limiting examples of suitable vectors include pAM.beta.1 and derivatives thereof (Renault et al., Gene 183:175-182 (1996); and O'Sullivan et al., Gene 137:227-231 (1993)); pMBB1 and pHW800, a derivative of pMBB1 (Wyckoff et al. Appl. Environ. Microbiol. 62:1481-1486 (1996)); pMG1, a conjugative plasmid (Tanimoto et al., J. Bacteriol. 184:5800-5804 (2002)); pNZ9520 (Kleerebezem et al., Appl. Environ. Microbiol. 63:4581-4584 (1997)); pAM401 (Fujimoto et al., Appl. Environ. Microbiol. 67:1262-1267 (2001)); and pAT392 (Arthur et al., Antimicrob. Agents Chemother. 38:1899-1903 (1994)). Expression vectors for E. faecalis using the nisA gene from Lactococcus may also be used (Eichenbaum et al., Appl. Environ. Microbiol. 64:2763-2769 (1998). Additionally, vectors for gene replacement in the E. faecium chromosome are used (Nallaapareddy et al., Appl. Environ. Microbiol. 72:334-345 (2006)).

Also, disruptions, including deletions, of one or more aldehyde dehydrogenases that convert 3-HP to its aldehydes may be made by methods known in the art, including but not limited to homologous recombination, may be used to target nucleotide regions upstream and downstream of a targeted aldehyde dehydrogenase (or portion thereof, i.e., a partial deletion) with a nucleic acid sequence having a selectable marker, or removal of a promoter (such as by similar homologous recombination) of such targeted aldehyde dehydrogenase. As noted for other species, genetic modification(s) directed to increase 3-HP production may also be provided in some embodiments.

For each of the General Prophetic Examples 9-15, the following 3-HP bio-production comparison may be incorporated thereto: Using analytical methods for 3-HP such as are described in Subsection III of Common Methods Section, below, 3-HP is obtained in a measurable quantity at the conclusion of a respective bio-production event conducted with the respective recombinant microorganism (see types of bio-production events, below, incorporated by reference into each respective General Prophetic Example). That measurable quantity is substantially greater than a quantity of 3-HP produced in a control bio-production event using a suitable respective control microorganism lacking the functional 3-HP pathway so provided in the respective General Prophetic Example. Tolerance improvements also may be assessed by any recognized comparative measurement technique, such as by using a MIC protocol provided in the Common Methods Section.

Common Methods Section

All methods in this Section are provided for incorporation into the above methods where so referenced therein and/or below.

Subsection I. Bacterial Growth Methods: Bacterial growth culture methods, and associated materials and conditions, are disclosed for respective species, that may be utilized as needed, as follows:

Acinetobacter calcoaceticus (DSMZ #1139) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended A. calcoaceticus culture are made into BHI and are allowed to grow for aerobically for 48 hours at 37° C. at 250 rpm until saturated.

Bacillus subtilis is a gift from the Gill lab (University of Colorado at Boulder) and is obtained as an actively growing culture. Serial dilutions of the actively growing B. subtilis culture are made into Luria Broth (RPI Corp, Mt. Prospect, Ill., USA) and are allowed to grow for aerobically for 24 hours at 37° C. at 250 rpm until saturated.

Chlorobium limicola (DSMZ#245) is obtained from the German Collection of Microorganisms and Cell Cultures (Braunschweig, Germany) as a vacuum dried culture. Cultures are then resuspended using Pfennig's Medium I and II (#28 and 29) as described per DSMZ instructions. C. limicola is grown at 25° C. under constant vortexing.

Citrobacter braakii (DSMZ #30040) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. braakii culture are made into BHI and are allowed to grow for aerobically for 48 hours at 30° C. at 250 rpm until saturated.

Clostridium acetobutylicum (DSMZ #792) is obtained from the German Collection of Microorganisms and Cell Cultures (Braunschweig, Germany) as a vacuum dried culture. Cultures are then resuspended in Clostridium acetobutylicum medium (#411) as described per DSMZ instructions. C. acetobutylicum is grown anaerobically at 37° C. at 250 rpm until saturated.

Clostridium aminobutyricum (DSMZ #2634) is obtained from the German Collection of Microorganisms and Cell Cultures (Braunschweig, Germany) as a vacuum dried culture. Cultures are then resuspended in Clostridium aminobutyricum medium (#286) as described per DSMZ instructions. C. aminobutyricum is grown anaerobically at 37° C. at 250 rpm until saturated.

Clostridium kluyveri (DSMZ #555) is obtained from the German Collection of Microorganisms and Cell Cultures (Braunschweig, Germany) as an actively growing culture. Serial dilutions of C. kluyveri culture are made into Clostridium kluyveri medium (#286) as described per DSMZ instructions. C. kluyveri is grown anaerobically at 37° C. at 250 rpm until saturated.

Cupriavidus metallidurans (DMSZ #2839) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. metallidurans culture are made into BHI and are allowed to grow for aerobically for 48 hours at 30° C. at 250 rpm until saturated.

Cupriavidus necator (DSMZ #428) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended C. necator culture are made into BHI and are allowed to grow for aerobically for 48 hours at 30° C. at 250 rpm until saturated. As noted elsewhere, previous names for this species are Alcaligenes eutrophus and Ralstonia eutrophus.

Desulfovibrio fructosovorans (DSMZ #3604) is obtained from the German Collection of Microorganisms and Cell Cultures (Braunschweig, Germany) as a vacuum dried culture. Cultures are then resuspended in Desulfovibrio fructosovorans medium (#63) as described per DSMZ instructions. D. fructosovorans is grown anaerobically at 37° C. at 250 rpm until saturated.

Escherichia coli Crooks (DSMZ#1576) is obtained from the German Collection of Microorganisms and Cell Cultures (Braunschweig, Germany) as a vacuum dried culture. Cultures are then resuspended in Brain Heart Infusion (BHI) Broth (RPI Corp, Mt. Prospect, Ill., USA). Serial dilutions of the resuspended E. coli Crooks culture are made into BHI and are allowed to grow for aerobically for 48 hours at 37° C. at 250 rpm until saturated.

Escherichia coli K12 is a gift from the Gill lab (University of Colorado at Boulder) and is obtained as an actively growing culture. Serial dilutions of the actively growing E. coli K12 culture are made into Luria Broth (RPI Corp, Mt. Prospect, Ill., USA) and are allowed to grow for aerobically for 24 hours at 37° C. at 250 rpm until saturated.

Halobacterium salinarum (DSMZ#1576) is obtained from the German Collection of Microorganisms and Cell Cultures (Braunschweig, Germany) as a vacuum dried culture. Cultures are then resuspended in Halobacterium medium (#97) as described per DSMZ instructions. H. salinarum is grown aerobically at 37° C. at 250 rpm until saturated.

Lactobacillus delbrueckii (#4335) is obtained from WYEAST USA (Odell, Oreg., USA) as an actively growing culture. Serial dilutions of the actively growing L. delbrueckii culture are made into Brain Heart Infusion (BHI) broth (RPI Corp, Mt. Prospect, Ill., USA) and are allowed to grow for aerobically for 24 hours at 30° C. at 250 rpm until saturated.

Metallosphaera sedula (DSMZ #5348) is obtained from the German Collection of Microorganisms and Cell Cultures (Braunschweig, Germany) as an actively growing culture. Serial dilutions of M. sedula culture are made into Metallosphaera medium (#485) as described per DSMZ instructions. M. sedula is grown aerobically at 65° C. at 250 rpm until saturated.

Propionibacterium freudenreichii subsp. shermanii (DSMZ#4902) is obtained from the German Collection of Microorganisms and Cell Cultures (Braunschweig, Germany) as a vacuum dried culture. Cultures are then resuspended in PYG-medium (#104) as described per DSMZ instructions. P. freudenreichii subsp. shermanii is grown anaerobically at 30° C. at 250 rpm until saturated.

Pseudomonas putida is a gift from the Gill lab (University of Colorado at Boulder) and is obtained as an actively growing culture. Serial dilutions of the actively growing P. putida culture are made into Luria Broth (RPI Corp, Mt. Prospect, Ill., USA) and are allowed to grow for aerobically for 24 hours at 37° C. at 250 rpm until saturated.

Streptococcus mutans (DSMZ#6178) is obtained from the German Collection of Microorganisms and Cell Cultures (Braunschweig, Germany) as a vacuum dried culture. Cultures are then resuspended in Luria Broth (RPI Corp, Mt. Prospect, Ill., USA). S. mutans is grown aerobically at 37° C. at 250 rpm until saturated.

Subsection II: Gel Preparation, DNA Separation, Extraction, Ligation, and Transformation Methods:

Molecular biology grade agarose (RPI Corp, Mt. Prospect, Ill., USA) is added to 1× TAE to make a 1% Agarose: TAE solution. To obtain 50× TAE add the following to 900 mL of distilled water: add the following to 900 ml distilled H₂O: 242 g Tris base (RPI Corp, Mt. Prospect, Ill., USA), 57.1 ml Glacial Acetic Acid (Sigma-Aldrich, St. Louis, Mo., USA) and 18.6 g EDTA (Fisher Scientific, Pittsburgh, Pa. USA) and adjust volume to 1 L with additional distilled water. To obtain 1× TAE, add 20 mL of 50× TAE to 980 mL of distilled water. The agarose-TAE solution is then heated until boiling occurred and the agarose is fully dissolved. The solution is allowed to cool to 50° C. before 10 mg/mL ethidium bromide (Acros Organics, Morris Plains, N.J., USA) is added at a concentration of 5 μl per 100 mL of 1% agarose solution. Once the ethidium bromide is added, the solution is briefly mixed and poured into a gel casting tray with the appropriate number of combs (Idea Scientific Co., Minneapolis, Minn., USA) per sample analysis. DNA samples are then mixed accordingly with 5× TAE loading buffer. 5× TAE loading buffer consists of 5× TAE (diluted from 50× TAE as described above), 20% glycerol (Acros Organics, Morris Plains, N.J., USA), 0.125% Bromophenol Blue (Alfa Aesar, Ward Hill, Mass., USA), and adjust volume to 50 mL with distilled water. Loaded gels are then run in gel rigs (Idea Scientific Co., Minneapolis, Minn., USA) filled with 1× TAE at a constant voltage of 125 volts for 25-30 minutes. At this point, the gels are removed from the gel boxes with voltage and visualized under a UV transilluminator (FOTODYNE Inc., Hartland, Wis., USA).

The DNA isolated through gel extraction is then extracted using the QIAquick Gel Extraction Kit following manufacturer's instructions (Qiagen (Valencia Calif. USA)). Similar methods are known to those skilled in the art.

The thus-extracted DNA then may be ligated into pSMART (Lucigen Corp, Middleton, Wis., USA), StrataClone (Stratagene, La Jolla, Calif., USA) or pCR2.1-TOPO TA (Invitrogen Corp, Carlsbad, Calif., USA) according to manufacturer's instructions. These methods are described in the next subsection of Common Methods.

Ligation Methods:

For Ligations into pSMART Vectors:

Gel extracted DNA is blunted using PCRTerminator (Lucigen Corp, Middleton, Wis., USA) according to manufacturer's instructions. Then 500 ng of DNA is added to 2.5 uL 4× CloneSmart vector premix, 1 ul CloneSmart DNA ligase (Lucigen Corp, Middleton, Wis., USA) and distilled water is added for a total volume of 10 ul. The reaction is then allowed to sit at room temperature for 30 minutes and then heat inactivated at 70° C. for 15 minutes and then placed on ice. E. cloni 10 G Chemically Competent cells (Lucigen Corp, Middleton, Wis., USA) are thawed for 20 minutes on ice. 40 ul of chemically competent cells are placed into a microcentrifuge tube and 1 ul of heat inactivated CloneSmart Ligation is added to the tube. The whole reaction is stirred briefly with a pipette tip. The ligation and cells are incubated on ice for 30 minutes and then the cells are heat shocked for 45 seconds at 42° C. and then put back onto ice for 2 minutes. 960 ul of room temperature Recovery media (Lucigen Corp, Middleton, Wis., USA) and places into microcentrifuge tubes. Shake tubes at 250 rpm for 1 hour at 37° C. Plate 100 ul of transformed cells on Luria Broth plates (RPI Corp, Mt. Prospect, Ill., USA) plus appropriate antibiotics depending on the pSMART vector used. Incubate plates overnight at 37° C.

For Ligations into StrataClone:

Gel extracted DNA is blunted using PCRTerminator (Lucigen Corp, Middleton, Wis., USA) according to manufacturer's instructions. Then 2 ul of DNA is added to 3 ul StrataClone Blunt Cloning buffer and 1 ul StrataClone Blunt vector mix amp/kan (Stratagene, La Jolla, Calif., USA) for a total of 6 ul. Mix the reaction by gently pipeting up at down and incubate the reaction at room temperature for 30 minutes then place onto ice. Thaw a tube of StrataClone chemically competent cells (Stratagene, La Jolla, Calif., USA) on ice for 20 minutes. Add 1 ul of the cloning reaction to the tube of chemically competent cells and gently mix with a pipette tip and incubate on ice for 20 minutes. Heat shock the transformation at 42° C. for 45 seconds then put on ice for 2 minutes. Add 250 ul pre-warmed Luria Broth (RPI Corp, Mt. Prospect, Ill., USA) and shake at 250 rpm for 37° C. for 2 hour. Plate 100 ul of the transformation mixture onto Luria Broth plates (RPI Corp, Mt. Prospect, Ill., USA) plus appropriate antibiotics. Incubate plates overnight at 37° C.

For Ligations into pCR2.1-TOPO TA:

Add 1 ul TOPO vector, 1 ul Salt Solution (Invitrogen Corp, Carlsbad, Calif., USA) and 3 ul gel extracted DNA into a microcentrifuge tube. Allow the tube to incubate at room temperature for 30 minutes then place the reaction on ice. Thaw one tube of TOP1OF′ chemically competent cells (Invitrogen Corp, Carlsbad, Calif., USA) per reaction. Add 1 ul of reaction mixture into the thawed TOP1OF′ cells and mix gently by swirling the cells with a pipette tip and incubate on ice for 20 minutes. Heat shock the transformation at 42° C. for 45 seconds then put on ice for 2 minutes. Add 250 ul pre-warmed SOC media (Invitrogen Corp, Carlsbad, Calif., USA) and shake at 250 rpm for 37° C. for 1 hour. Plate 100 ul of the transformation mixture onto Luria Broth plates (RPI Corp, Mt. Prospect, Ill., USA) plus appropriate antibiotics. Incubate plates overnight at 37° C.

General Transformation and Related Culture Methodologies:

Chemically competent transformation protocols are carried out according to the manufacturer's instructions or according to the literature contained in Molecular Cloning (Sambrook and Russell, 2001). Generally, plasmid DNA or ligation products are chilled on ice for 5 to 30 min. in solution with chemically competent cells. Chemically competent cells are a widely used product in the field of biotechnology and are available from multiple vendors, such as those indicated above in this Subsection. Following the chilling period cells generally are heat-shocked for 30 seconds at 42° C. without shaking, re-chilled and combined with 250 microliters of rich media, such as S.O.C. Cells are then incubated at 37° C. while shaking at 250 rpm for 1 hour. Finally, the cells are screened for successful transformations by plating on media containing the appropriate antibiotics.

Alternatively, selected cells may be transformed by electroporation methods such as are known to those skilled in the art.

The choice of an E. coli host strain for plasmid transformation is determined by considering factors such as plasmid stability, plasmid compatibility, plasmid screening methods and protein expression. Strain backgrounds can be changed by simply purifying plasmid DNA as described above and transforming the plasmid into a desired or otherwise appropriate E. coli host strain such as determined by experimental necessities, such as any commonly used cloning strain (e.g., DH5α, Top1OF′, E. cloni 10 G, etc.).

To Make 1L M9 Minimal Media:

M9 minimal media was made by combining 5× M9 salts, 1M MgSO₄, 20% glucose, 1M CaCl₂ and sterile deionized water. The 5× M9 salts are made by dissolving the following salts in deionized water to a final volume of 1 L: 64 g Na₂HPO₄.7H₂O, 15 g KH₂PO₄, 2.5 g NaCl, 5.0 g NH₄Cl. The salt solution was divided into 200 mL aliquots and sterilized by autoclaving for 15 minutes at 15 psi on the liquid cycle. A 1M solution of MgSO₄ and 1M CaCl₂ were made separately, then sterilized by autoclaving. The glucose was filter sterilized by passing it thought a 0.22 μm filter. All of the components are combined as follows to make 1 L of M9: 750 mL sterile water, 200 mL 5× M9 salts, 2 mL of 1M MgSO₄, 20 mL 20% glucose, 0.1 mL CaCl₂, Q.S. to a final volume of 1 L.

To Make EZ Rich Media:

All media components were obtained from TEKnova (Hollister Calif. USA) and combined in the following volumes. 100 mL 10× MOPS mixture, 10 mL 0.132M K₂ HPO_{4,} 100 mL 10× ACGU, 200 mL 5× Supplement EZ, 10 mL 20% glucose, 580 mL sterile water.

Subsection IIIa. 3-HP Preparation

A 3-HP stock solution was prepared as follows and used in examples other than Example 1. A vial of β-propriolactone (Sigma-Aldrich, St. Louis, Mo., USA) was opened under a fume hood and the entire bottle contents was transferred to a new container sequentially using a 25-mL glass pipette. The vial was rinsed with 50 mL of HPLC grade water and this rinse was poured into the new container. Two additional rinses were performed and added to the new container. Additional HPLC grade water was added to the new container to reach a ratio of 50 mL water per 5 mL β-propriolactone. The new container was capped tightly and allowed to remain in the fume hood at room temperature for 72 hours. After 72 hours the contents were transferred to centrifuge tubes and centrifuged for 10 minutes at 4,000 rpm. Then the solution was filtered to remove particulates and, as needed, concentrated by use of a rotary evaporator at room temperature. Assay for concentration was conducted per below, and dilution to make a standard concentration stock solution was made as needed.

It is noted that there appear to be small lot variations in the toxicity of 3-HP solutions. Without being bound to a particular theory, it is believed the variation can be correlated with a low level of contamination by acrylic acid, which is more toxic than 3-HP, and also, to a lesser extent, to presence of a polymer of β-propriolactone. HPLC results show the presence of the acrylic peak, which, as noted, is a minor contaminant varying in concentration from batch to batch.

Subsection IIIb. HPLC and GC/NIS Analytical Methods for Detection of 3-HP and its Metabolites

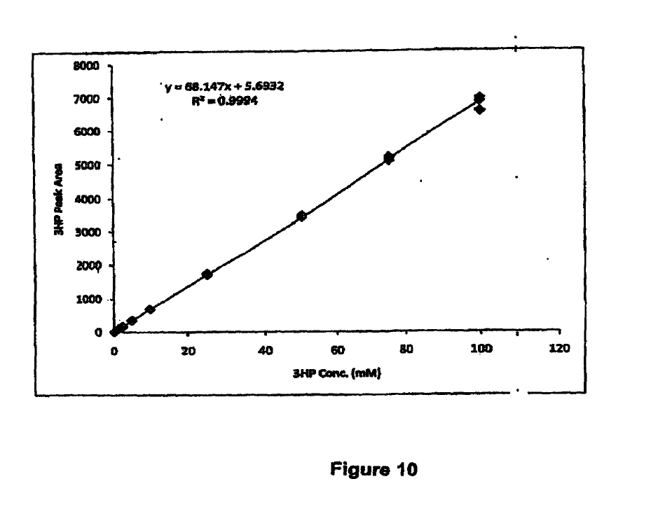

For HPLC analysis of 3-HP, and metabolites of Example 1, the Waters chromatography system (Milford, Mass.) consisted of the following: 600S Controller, 616 Pump, 717 Plus Autosampler, 486 Tunable UV Detector, and an in-line mobile phase Degasser. In addition, an Eppendorf external column heater is used and the data are collected using an SRI (Torrance, Calif.) analog-to-digital converter linked to a standard desk top computer. Data are analyzed using the SRI Peak Simple software. A Coregel 64H ion exclusion column (Transgenomic, Inc., San Jose, Calif.) is employed. The column resin is a sulfonated polystyrene divinyl benzene with a particle size of 10 μm and column dimensions are 300×7.8 mm The mobile phase consisted of sulfuric acid (Fisher Scientific, Pittsburgh, Pa. USA) diluted with deionized (18 MΩcm) water to a concentration of 0.02 N and vacuum filtered through a 0.2 μm nylon filter. The flow rate of the mobile phase is 0.6 mL/min. The UV detector is operated at a wavelength of 210 nm and the column is heated to 60° C. The same equipment and method as described herein is used for 3-HP analyses for relevant prophetic examples. Calibration curves using this HPLC method with a 3-HP standard (TCI America, Portland, Oreg.) is provided in FIG. 10.

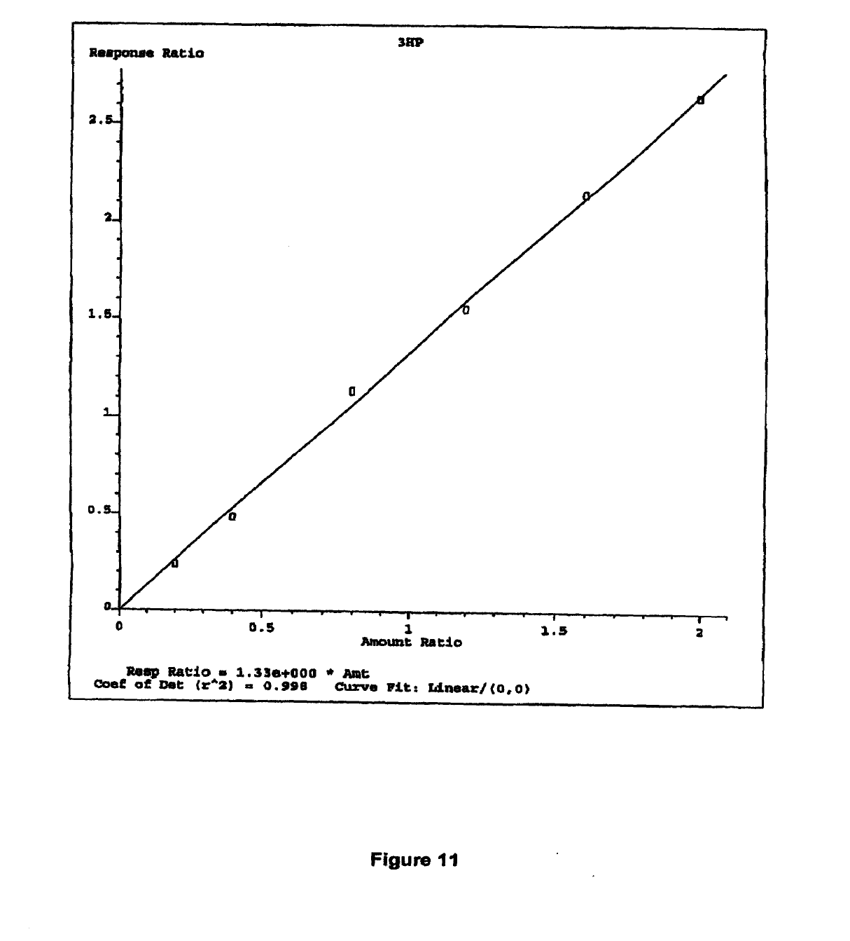

The following method is used for GC-MS analysis of 3-HP. Soluble monomeric 3-HP is quantified using GC-MS after a single extraction of the fermentation media with ethyl acetate. The GC-MS system consists of a Hewlett Packard model 5890 GC and Hewlett Packard model 5972 MS. The column is Supelco SPB-1 (60 m×0.32 mm×0.25 μm film thickness). The capillary coating is a non-polar methylsilicone. The carrier gas is helium at a flow rate of 1 mL/min. 3-HP is separated from other components in the ethyl acetate extract, using a temperature gradient regime starting with 40° C. for 1 minute, then 10° C./minute to 235° C., and then 50° C./minute to 300° C. Tropic acid (1 mg/mL) is used as the internal standard. 3-HP is quantified using a 3HP standard curve at the beginning of the run and the data are analyzed using HP Chemstation. A calibration curve, automatically generated with use of a standard, is provided as FIG. 11.

The following method is used for GC-MS analysis of metabolites of 3-HP. The metabolites are quantified using GC-MS after a single extraction of the fermentation media with ethyl acetate and derivatization with BSTFA. The GC-MS system consists of a Hewlett Packard model 5890 GC and Hewlett Packard model 5972 MS. The column is Supelco SPB-1 (60 m×0.32 mm×0.25 μm film thickness). The capillary coating is a non-polar methylsilicone. The carrier gas is helium at a flow rate of 1 mL/min. The metabolites are separated using a temperature gradient regime starting at 100° C. for 1 minute, then 10° C./minute to 235° C., and then 50° C./minute to 300° C. Tropic acid (1 mg/mL) is used as the internal standard. The metabolites are quantified using standard curves generated for each metabolite from a mixture of at the beginning of the run and the data are analyzed using HP Chemstation.

Subsection IV: Methods for Example 1

3-HP Metabolite Studies.

Cultures of strains of Example 1 were initiated in 5 mL, LB+antibiotic where appropriate and were grown at 37 C overnight in a shaking incubator. The next day, 250 uL of the overnight cultures were inoculated into 25 mL of M9+kanamycin. This culture was incubated at 37 C to OD₆₀₀˜0.4 (approx 6-8 hours). After 6-8 hours, the cells were centrifuged for 10 minutes at 4 C and the cell pellet was re-suspended in 1 mL M9 minimal media. These cells were used to provide a constant inoculum into respective 10 mL test volumes of M9 minimal medium (9.5 mL M9+500 μL of the re-suspended culture) plus 20 g/L 3-HP, and with putrescine (0.1 g/L, MP Biomedicals) where indicated. Culture tubes containing these respective test volumes, and also control culture tubes, were incubated for 20 hours at 37 C in a shaking incubator. The culture tube volumes were centrifuged for 10 minutes at 4 C and 0.7 mL of each supernatant was syringe filtered into an HPLC collection vial. The rest of the supernatant was removed and the cell pellet was rinsed with M9. Each cell pellet was then re-suspended in 1 mL M9 and incubated at room temperature for approximately an hour. Then all cell pellets were sonicated for 30 seconds at 83% amplitude. The sonicated cells were then centrifuged again for 10 minutes at 4 C. The sample supernatant (0.7 mL) was then syringe filtered into an HPLC collection vial. All the intracellular and extracellular metabolites were analyzed by HPLC as described in the Common Methods Section, Subsection III. The presence of an aldehyde (which was previously identified as 3HPA) was identified as a novel peak in routine HPLC analysis which was isolated by fractionation and characterized as an aldehyde with the aldehyde detection reagent Purpald® following manufacturer's instructions. Although this peak has an elution time very similar to lactic acid, the absence of lactic acid was confirmed both with enzymatic assay and GC/MS analysis.

Summary of Suppliers Section

This section is provided for a summary of suppliers, and may be amended to incorporate additional supplier information in subsequent filings. The names and city addresses of major suppliers are provided in the methods above. In addition, as to Qiagen products, the DNeasy® Blood and Tissue Kit, Cat. No. 69506, is used in the methods for genomic DNA preparation; the QIAprep® Spin (“mini prep”), Cat. No. 27106, is used for plasmid DNA purification, and the QIAquick® Gel Extraction Kit, Cat. No. 28706, is used for gel extractions as described above.

TABLE 1

| | | | SEQ ID |
|---|---|---|---|
| | | SEQ ID | NO. by |
| | | NO. of | Gene |
| Gene | Gene Product | Gene | Product |
| aldA | aldehyde dehydrogenase A | 001 | 023 |
| aldB | acetaldehyde dehydrogenase | 002 | 024 |
| betB | betaine aldehyde dehydrogenase | 003 | 025 |
| eutE | predicted aldehyde dehydrogenase | 004 | 026 |
| eutG | predicted alcohol dehydrogenase in | 005 | 027 |
| | ethanolamine utilization | | |
| fucO | L-1,2-propanediol oxidoreductase | 006 | 028 |
| gabD | succinate semialdehyde dehydrogenase | 007 | 029 |
| garR | tartronate semialdehyde reductase | 008 | 030 |
| gldA | D-aminopropanol dehydrogenase/glycerol | 009 | 031 |
| | dehydrogenase | | |
| glxR | tartronate semialdehyde reductase 2 | 010 | 032 |
| gnd | 6-phosphogluconate dehydrogenase | 011 | 033 |
| | (decarboxylating) | | |
| ldhA | D-lactate dehydrogenase | 012 | 034 |
| maoC | putative ring-cleavage enzyme of | 013 | 035 |
| | phenylacetate degradation | | |
| proA | glutamate-5-semialdehyde dehydrogenase | 014 | 036 |
| putA | fused PutA transcriptional represser/ | 015 | 037 |
| | proline dehydrogenase/1-pyrroline-5- | | |
| | carboxylate dehydrogenase | | |
| puuC | γ-glutamyl-γ-aminobutyraldehyde | 016 | 038 |
| | dehydrogenase | | |
| sad/yneI | succinate semialdehyde dehydrogenase, | 017 | 039 |
| | NAD⁺-dependent | | |
| ssuD | afkanesulfonate monooxygenase | 018 | 040 |
| ybdH | predicted oxidoreductase | 019 | 041 |
| ydcW | γ-aminobutyraldehyde dehydrogenase | 020 | 042 |
| ygbJ | predicted dehydrogenase | 021 | 043 |
| yiaY | predicted Fe-containing alcohol | 022 | 044 |
| | dehydroqenase | | |

TABLE 2

| | | | | | | | |
|---|---|---|---|---|---|---|---|
| Coli | | Gene | | Gene | | Gene | |
| Gene | | Symbol | e_value | Symbol | e_value | Symbol | e_value |
| Symbol | Product | B. subtilis | B. subtilis | S. cerevisiae | S. cerevisia | C. necator | C. necator |

Homology Relationships for Genetic Elements of E. coli Aldeheyde Dehydrogenase

| | | | | | | | |
|---|---|---|---|---|---|---|---|
| adhE | fused acetaldehyde-CoA | gbsB | 1.00E−29 | YGL256W | 8.00E−36 | h16_A0861 | 9.00E−30 |
| | dehydrogenase/iron-dependent | | | | | | |
| | alcohol dehydrogenase/pyruvate- | | | | | | |
| | formate lyase dea | | | | | | |
| adhE | fused acetaldehyde-CoA | yugK | 2.00E−14 | YGL256W | 8.00E−36 | gbd | 2.00E−23 |
| | dehydrogenase/iron-dependent | | | | | | |
| | alcohol dehydrogenase/pyruvate- | | | | | | |
| | formate lyase dea | | | | | | |
| adhE | fused acetaldehyde-CoA | yugJ | 2.00E−13 | YGL256W | 8.00E−36 | h16_A2747 | 7.00E−63 |
| | dehydrogenase/iron-dependent | | | | | | |
| | alcohol dehydrogenase/pyruvate- | | | | | | |
| | formate lyase dea | | | | | | |
| adhE | fused acetaldehyde-CoA | yugJ | 2.00E−13 | YGL256W | 8.00E−36 | h16_B0831 | 2.00E−14 |
| | dehydrogenase/iron-dependent | | | | | | |
| | alcohol dehydrogenase/pyruvate- | | | | | | |
| | formate lyase dea | | | | | | |
| adhE | fused acetaldehyde-CoA | yugJ | 2.00E−13 | YGL256W | 8.00E−36 | pcpE | 1.00E−14 |
| | dehydrogenase/iron-dependent | | | | | | |
| | alcohol dehydrogenase/pyruvate- | | | | | | |
| | formate lyase dea | | | | | | |
| adhP | ethanol-active dehydrogenase/ | gutB | 2.00E−24 | YBR145W | 4.00E−44 | adh | 4.00E−17 |
| | acetaldehyde-active reductase | | | | | | |
| adhP | ethanol-active dehydrogenase/ | yjmD | 4.00E−18 | YMR303C | 1.00E−43 | tdh | 3.00E−18 |
| | acetaldehyde-active reductase | | | | | | |
| adhP | ethanol-active dehydrogenase/ | tdh | 3.00E−18 | YOL086C | 4.00E−41 | 38637893 | 2.00E−27 |
| | acetaldehyde-active reductase | | | | | | |
| adhP | ethanol-active dehydrogenase/ | yogA | 2.00E−11 | YMR083W | 5.00E−41 | h16_B0517 | 7.00E−14 |
| | acetaldehyde-active reductase | | | | | | |

Homology Relationships for Genetic Elements of ALD

| | | | | | | | |
|---|---|---|---|---|---|---|---|
| adhP | ethanol-active dehydrogenase/ | adhB | 4.00E−13 | YDL168W | 4.00E−21 | adhC | 4.00E−21 |
| | acetaldehyde-active reductase | | | | | | |
| adhP | ethanol-active dehydrogenase/ | adhA | 2.00E−34 | YCR105W | 1.00E−19 | adhP | 5.00E−29 |
| | acetaldehyde-active reductase | | | | | | |
| adhP | ethanol-active dehydrogenase/ | adhA | 2.00E−34 | YMR318C | 6.00E−18 | h16_B1734 | 2.00E−12 |
| | acetaldehyde-active reductase | | | | | | |
| adhP | ethanol-active dehydrogenase/ | adhA | 2.00E−34 | YAL060W | 2.00E−14 | h16_B1745 | 4.00E−24 |
| | acetaldehyde-active reductase | | | | | | |

. . . (intervening data removed to shorten table)

| | | | | | | | |
|---|---|---|---|---|---|---|---|
| yiaY | predicted Fe-containing alcohol | yugJ | 4.00E−26 | YGL256W | 5.00E−118 | h16_B0831 | 3.00E−27 |
| | dehydrogenase | | | | | | |
| yiaY | predicted Fe-containing alcohol | yugJ | 4.00E−26 | YGL256W | 5.00E−118 | pcpE | 1.00E−25 |
| | dehydrogenase | | | | | | |
| yiaY | predicted Fe-containing alcohol | yugJ | 4.00E−26 | YGL256W | 5.00E−118 | h16_B1417 | 6.00E−13 |
| | dehydrogenase | | | | | | |
| yqhD | alcohol dehydrogenase, NAD(P)- | gbsB | 5.00E−18 | YGL256W | 9.00E−19 | h16_A0861 | 2.00E−20 |
| | dependent | | | | | | |
| yqhD | alcohol dehydrogenase, NAD(P)- | yugK | 9.00E−67 | YGL256W | 9.00E−19 | gbd | 3.00E−24 |
| | dependent | | | | | | |
| yqhD | alcohol dehydrogenase, NAD(P)- | yugJ | 7.00E−73 | YGL256W | 9.00E−19 | h16_B0831 | 1.00E−12 |
| | dependent | | | | | | |

TABLE 3

| | | Forward | | Reverse |
|---|---|---|---|---|
| | | Primer | | Primer |
| | Forward | SEQ ID | Reverse | SEQ ID |
| Gene | Primer | NO. | Primer | NO. |
| adhE | ATGGCTGTTA | 045 | AGCGGATTTTTTCG | 046 |
| | CTAATGTCGC | | CTTTTTTCTC | |
| adhP | ATGAAGGCTG | 047 | GTGACGGAAATCAA | 048 |
| | CAGTTGTTAC | | TCACC | |
| aldA | ATGTCAGTACCC | 049 | AGACTGTAAATAAA | 050 |
| | GTTCAAC | | CCACCTGG | |
| aldB | ATGACCAATAATC | 051 | GAACAGCCCCAACG | 052 |
| | CCCCTTCA | | | |
| astD | ATGACTTTATGGA | 053 | TCGCACCACCTCAT | 054 |
| | TTAACGGTGAC | | C | |
| betB | ATGTCCCGAATG | 055 | GAATATGGACTGGA | 056 |
| | GCAGAAC | | ATTTAGCC | |
| dkgA | ATGGCTAATCCA | 057 | GCCGCCGAACTGG | 058 |
| | ACCGTTATTAAGC | | TC | |
| dkgB | ATGGCTATCCCT | 059 | ATCCCATTCAGGAG | 060 |
| | GCATTTGG | | CCAGA | |
| eutE | ATGAATCAACAG | 061 | AACAATGCGAAACG | 062 |
| | GATATTGAACAG | | CATCG | |
| eutG | ATGCAAAATGAAT | 063 | TTGCGCCGCTGCGT | 064 |
| | TGCAGACCG | | A | |
| feaB | ATGACAGAGCCG | 065 | ATACCGTACACACA | 066 |
| | CATGTA | | CCGAC | |
| fucO | ATGATGGCTAAC | 067 | CCAGGCGGTATGGT | 068 |
| | AGAATGATTCTG | | AAAG | |
| gabD | ATGAAACTTAACG | 069 | AAGACCGATGCACA | 070 |
| | ACAGTAACTTAT | | TATAT | |
| garR | ATGACTATGAAA | 071 | ACGAGTAACTTCGA | 072 |
| | GTTGGTTTTATTG | | CTTTC | |
| gldA | ATGGACCGCATT | 073 | TTCCCACTCTTGCA | 074 |
| | ATTCAATC | | GGAAAC | |
| glxR | ATGAAACTGGGA | 075 | GGCCAGTTTATGGT | 076 |
| | TTTATTGGCTTAG | | TAGCC | |
| gnd | ATGTCCAAGCAA | 077 | ATCCAGCCATTCGG | 078 |
| | CAGATCGG | | TATGG | |
| IdhA | ATGAAACTCGCC | 079 | AACCAGTTCGTTCG | 080 |
| | GTTTATAGC | | GGC | |
| maoC | ATGCAGCAGTTA | 081 | ATCGACAAAATCAC | 082 |
| | GCCAGTTTC | | CGTGCTG | |
| proA | ATGCTGGAACAA | 083 | CGCACGAATGGTGT | 084 |
| | ATGGGCAT | | AATC | |
| putA | ATGGGAACCACC | 085 | ACCTATAGTCATTA | 086 |
| | ACCATG | | AGCTGGCG | |
| puuC | ATGAATTTTCATC | 087 | GGCCTCCAGGCTTA | 088 |
| | ATCTGGCTTAC | | TCC | |
| sad | ATGACCATTACTC | 089 | AGATCCGGTCTTTC | 090 |
| | CGGCAAC | | CACAC | |
| sdaA | ATGATTAGTCTAT | 091 | GTCACACTGGACTT | 092 |
| | TCGACATGTTA | | TGATTG | |
| sdAB | ATGATTAGCGTAT | 093 | ATCGCAGGCAACGA | 094 |
| | TCGATATTTTC | | TCTTC | |
| ssuD | ATGAGTCTGAATA | 095 | GCTTTGCGCGACTT | 096 |
| | TGTTCTGGTT | | TACG | |
| tdcB | ATGCATATTACAT | 097 | AGCGTCAACGAAAC | 098 |
| | ACGATCTGC | | CGGT | |
| tdcG | ATGATTAGTGCAT | 099 | GCCGCAGACCACTT | 100 |
| | TCGATATTTTC | | TAAT | |
| usg | ATGTCTGAAGGC | 101 | GTACAGATACTCCT | 102 |
| | TGGAACAT | | GCACC | |
| ybdH | ATGCCTCACAAT | 103 | GGCTTTAAACGATT | 104 |
| | CCTATCCG | | CCACTT | |
| ydcW | ATGCAACATAAGT | 105 | TACAAATTGGTACT | 106 |
| | TACTGATTAACG | | GCACCG | |
| yeaE | ATGCAACAAAAAA | 107 | CACCATATCCAGCG | 108 |
| | TGATTCAATTTAG | | CAGTT | |
| ygbJ | ATGAAAACGGGA | 109 | TGATTTCGCTCCCG | 110 |
| | TCTGAGTTTC | | GTAG | |
| yghD | ATGTTACGCGAT | 111 | CCCCCGTCCAAACT | 112 |
| | AAATTTATTCAC | | CCAG | |
| yghZ | ATGGTCTGGTTA | 113 | TTTATCGGAAGACG | 114 |
| | GCGAATCC | | CCTGC | |
| yiaY | ATGGCAGCTTCA | 115 | CATCGCTGCGCGAT | 116 |
| | ACGTTCTT | | AAATC | |
| yqhD | ATGAACAACTTTA | 117 | GCGGGCGGCTTCG | 118 |
| | ATCTGCACAC | | TATATA | |

TABLE 4

| Strain Name | Genotype (each gene below is deleted) |
|---|---|
| BX_00106.0 | ldhA, pflB, fruR |
| BX_00150.0 | ldhA, pflB, fruR, aldA |
| BX_00153.0 | ldhA, pflB, fruR, aldB |
| BX_00151.0 | ldhA, pflB, fruR, puuC |
| BX_00165.0 | ldhA, pflB, fruR, aldA, aldB |
| BX_00157.0 | ldhA, pflB, fruR, puuC, aldA |
| BX_00155.0 | ldhA, pflB, fruR, puuC, aldB |
| BX_00169.0 | ldhA, pflB, fruR, puuC, aldB, aldA |

TABLE 5

| | | SEQ | |
|---|---|---|---|
| Primer | Primer Sequence | ID | Primer |
| Name | (5′ → 3′) | No. | Description |
| CPM0303 | GAGCACAGTATCGCAAACATG | 136 | pflB 300 |
| | | | upstream |
| CPM0304 | CAGGCAGCGCATCAGGCAGCCC | 137 | pflB 300 |
| | TGG | | downstream |
| CPM0307 | AGCAGGCACCAGCGGTAAGC | 138 | fruR 300 |
| | TTG | | upstream |
| CPM0308 | AACAGTCCTTGTTACGTCTGTGT | 139 | fruR 300 |
| | GG | | downstream |
| KEIO_0015 | AAAATTGCCCGTTTGTGAACCAC | 140 | aldA 300 |
| | | | upstream |
| KEIO_0016 | ATCATTGGCAGCCATTTCGGTTC | 141 | aldA 300 |
| | | | downstream |
| KEIO_0017 | GAAATTGTGGCGATTTATCGCGC | 142 | aldB 300 |
| | | | upstream |
| KEIO_0018 | CCCAGAAACGTACTTCTGTTGGC | 143 | aldB 300 |
| | G | | downstream |
| Keio_0007 | GGCGGCAAGTGAGCGAATCC CG | 144 | puuC_up- |
| | | | stream |
| Keio_0008 | CGCTTGCGCCAAAGCCGATGCG | 145 | puuC_down- |
| | | | stream |

TABLE 6

| | | SEQ | |
|---|---|---|---|
| Primer | Primer Sequence | ID | Primer |
| Name | (5′ → 3′) | No. | Description |
| Keio_0075 | TTTATCGATA TTGATCCAGG TG | 134 | ldhA 600 |
| | | | upstream |
| Keio_0076 | GTGTGCATTACCCAACGGCAAACG | 135 | ldhA 600 |
| | | | downstream |
| Keio_0077 | ATCACCTGGG GTCAGTTGGC G | 136 | pflB 600 |
| | | | upstream |
| Keio_0078 | CGTCGTTCATCTGTTTGAGATCG | 137 | pflB 600 |
| | | | downstream |
| Keio_0083 | CCAGCGTGGC TACAACATTG AAA | 138 | fruR 600 |
| | | | upstream |
| Keio_0084 | TCCCACTGAAAGGAGTTTACGG | 139 | fruR 600 |
| | | | downstream |
| Keio_0079 | GCATCGCGCT ATTGAATCAG | 140 | aldA 600 |
| | GCCG | | upstream |
| Keio_0080 | CGTCATGCACCACTAACTGTCTTG | 141 | aldA 600 |
| | | | downstream |
| Keio_0081 | GCGTGAAGCA ATGGCTTATG | 142 | aldB 600 |
| | CCCA | | upstream |
| Keio_0082 | CAAAAATAAGCACTCCCAGTGC | 143 | aldB 600 |
| | | | downstream |
| Keio_0007 | GGCGGCAAGTGAGCGAATCC CG | 144 | puuC_ |
| | | | upstream |
| Keio_0008 | CGCTTGCGCCAAAGCCGATGCG | 145 | puuC_ |
| | | | downstream |
| K1* | CAGTCATAGCCGAATAGCCT | 146 | Kanamycin |
| | | | internal |

1. A method of making a genetically modified microorganism comprising:
a. providing to a selected microorganism at least one genetic modification of a 3-hydroxypropionic acid (“3-HP”) production pathway to increase microbial synthesis of 3-HP above the rate of a control microorganism lacking the at least one genetic modification; and
b. providing to the selected microorganism at least one genetic modification to each of two, three, four, five, or more aldehyde dehydrogenases that function to convert 3-HP to an aldehyde of 3-HPxx.

2. The method of claim 1, wherein the aldehyde of 3-HP is malonate semialdehyde or 3-hydroxypropionaldehyde.

3. The method of claim 1, step a comprising providing a nucleic acid sequence encoding malonyl Co-A reductase.

4. The method of claim 1, step a comprising providing a nucleic acid sequence encoding a 3-hydroxyacid dehydrogenase.

5. (canceled)

6. The method of claim 1, step a comprising providing a nucleic acid sequence encoding a β-alanine aminotransferase.

7. The method of claim 1, step a comprising providing a nucleic acid sequence encoding an alanine-2,3-aminotransferase.

8. The method of claim 1, step a comprising providing a nucleic acid sequence encoding an oxaloacetate α-decarboxylase.

9. The method of claim 1, step a comprising providing a nucleic acid sequence encoding a glycerol dehydratase.

10. The method of claim 1, step a comprising providing a nucleic acid sequence encoding a 3-phoshpoglycerate phosphatase.

11. The method of claim 1, step a comprising providing a nucleic acid sequence encoding a glycerate dehydratase.

12. The method of claim 1, step a comprising providing a nucleic acid sequence encoding a β-alanine aminotransferase.

13. The method of claim 1, wherein the genetic modifications of step b reduce conversion of 3-HP to the aldehyde of 3-HP.

14-37. (canceled)

38. The method of claim 1, additionally comprising disrupting a nucleic acid sequence encoding lactate dehydrogenase.

39. The method of claim 1, wherein the selected microorganism comprises a disruption of a nucleic acid sequence encoding lactate dehydrogenase.

40-84. (canceled)

85. A genetically modified microorganism comprising:
a. at least one genetic modification to produce 3-hydroxypropionic acid (“3-HP”); and
b. at least one genetic modification to each of at least two aldehyde dehydrogenases effective to decrease each said aldehyde dehydrogenase's respective enzymatic activity and effective to decrease metabolism of 3-HP to any aldehydes of 3-HP,
as compared to the metabolism of a control microorganism lacking the at least two genetic modifications of the aldehyde dehydrogenases.

86. The genetically modified microorganism of claim 85, the at least one genetic modification to produce 3-HP comprising at least one heterologous nucleic acid sequence encoding an enzyme in a 3-HP production pathway, the enzyme selected from the group consisting of malonyl Co-A reductase, 3-hydroxyacid dehydrogenase, β-alanine aminotransferase alanine-2,3-aminotransferase oxaloacetate α-decarboxylase, glycerol dehydratase, 3-phoshpoglycerate phosphatase, and glycerate dehydratase.

87. The genetically modified microorganism of claim 85, wherein step b comprises introducing to the microorganism at least one genetic modification of a nucleic acid sequence encoding an enzyme that is within a 50, 60, 70, 80, 90, or 95 percent identity of one of the aldehyde dehydrogenase amino acid sequences of Table 1.

88. The genetically modified microorganism of claim 85, wherein the microorganism comprises a disruption of a nucleic acid sequence encoding lactate dehydrogenase.

89-106. (canceled)

107. A genetically modified microorganism comprising at least one genetic modification of each of two or more aldehyde dehydrogenases, said aldehyde dehydrogenases capable of converting 3-hydroxypropionic acid (“3-HP”) to any of its aldehyde metabolites.

108-125. (canceled)

126. A genetically modified microorganism comprising at least one genetic modification of each of at least two aldehyde dehydrogenases effective to decrease microbial enzymatic conversion of 3-hydroxypropionic acid (“3-HP”) to an aldehyde of 3-HP as compared to the enzymatic conversion of a control microorganism lacking the genetic modifications, wherein the genetically modified microorganism comprises additional genetic modification(s) to increase 3-HP production.

127-140. (canceled)

141. The genetically modified microorganism of claim 126, wherein the genetically modified microorganism comprises a disruption of a nucleic acid sequence encoding lactate dehydrogenase.

142-157. (canceled)

158. A culture system comprising:
a. a population of a genetically modified microorganism of claim 85; and
b. a media comprising nutrients for the population.
